SEQUENCE LISTING (123 sequences)

SEQ ID NO: 1 - RNA, 21 nt, synthetic construct
  source 1..21: mol_type = other RNA
caggagttcgaagtgatggaa

SEQ ID NO: 2 - RNA, 21 nt, synthetic construct
  source 1..21: mol_type = other RNA
tacaccatgcaccaagaccaa

SEQ ID NO: 3 - RNA, 21 nt, synthetic construct
  source 1..21: mol_type = other RNA
cggacgctggcctgaaagaat

SEQ ID NO: 4 - RNA, 21 nt, synthetic construct
  source 1..21: mol_type = other RNA
gatgctaagagcactccaaca

SEQ ID NO: 5 - RNA, 21 nt, synthetic construct
  source 1..21: mol_type = other RNA
tggatttcctctccaaagtta

SEQ ID NO: 6 - RNA, 21 nt, synthetic construct
  source 1..21: mol_type = other RNA
tcacgtttcacgtggaaatca

SEQ ID NO: 7 - RNA, 21 nt, synthetic construct
  source 1..21: mol_type = other RNA
ggactggaagcgatgacaaaa

SEQ ID NO: 8 - RNA, 21 nt, synthetic construct
  source 1..21: mol_type = other RNA
ggaaggtgcagataattaata

SEQ ID NO: 9 - RNA, 21 nt, synthetic construct
  source 1..21: mol_type = other RNA
aggtgcagataattaataaga

SEQ ID NO: 10 - RNA, 21 nt, synthetic construct
  source 1..21: mol_type = other RNA
gcagataattaataagaagca

SEQ ID NO: 11 - RNA, 21 nt, synthetic construct
  source 1..21: mol_type = other RNA
tcttagcaacgtccagtccaa

SEQ ID NO: 12 - RNA, 21 nt, synthetic construct
  source 1..21: mol_type = other RNA
aagtgtggctcaaaggataat

SEQ ID NO: 13 - RNA, 21 nt, synthetic construct
  source 1..21: mol_type = other RNA
ggctcaaaggataatatcaaa

SEQ ID NO: 14 - RNA, 21 nt, synthetic construct
  source 1..21: mol_type = other RNA
ctcaaaggataatatcaaaca

SEQ ID NO: 15 - RNA, 21 nt, synthetic construct
  source 1..21: mol_type = other RNA
cagtgtgcaaatagtctacaa

SEQ ID NO: 16 - RNA, 21 nt, synthetic construct
  source 1..21: mol_type = other RNA
gtggccaggtggaagtaaaat

SEQ ID NO: 17 - RNA, 21 nt, synthetic construct
  source 1..21: mol_type = other RNA
tggccaggtggaagtaaaata

SEQ ID NO: 18 - RNA, 21 nt, synthetic construct
  source 1..21: mol_type = other RNA
cggaggaaataaaaagattga

SEQ ID NO: 19 - RNA, 21 nt, synthetic construct
  source 1..21: mol_type = other RNA
ggaggaaataaaaagattgaa

SEQ ID NO: 20 - RNA, 21 nt, synthetic construct
  source 1..21: mol_type = other RNA
gaggaaataaaaagattgaaa

SEQ ID NO: 21 - RNA, 21 nt, synthetic construct
  source 1..21: mol_type = other RNA
tggccaagcagggtttgtgat

SEQ ID NO: 22 - RNA, 21 nt, synthetic construct
  source 1..21: mol_type = other RNA
gctcgggacttcaaaatcagt

SEQ ID NO: 23 - RNA, 21 nt, synthetic construct
  source 1..21: mol_type = other RNA
ggcaattccttttgattcttt

SEQ ID NO: 24 - RNA, 21 nt, synthetic construct
  source 1..21: mol_type = other RNA
agcaacaaaggatttgaaact

SEQ ID NO: 25 - RNA, 21 nt, synthetic construct
  source 1..21: mol_type = other RNA
gcaacaaaggatttgaaactt

SEQ ID NO: 26 - RNA, 21 nt, synthetic construct
  source 1..21: mol_type = other RNA
tccacagaaaccctgttttat

SEQ ID NO: 27 - RNA, 21 nt, synthetic construct
  source 1..21: mol_type = other RNA
ccacagaaaccctgttttatt

SEQ ID NO: 28 - RNA, 21 nt, synthetic construct
  source 1..21: mol_type = other RNA
ctccatactgagggtgaaatt

SEQ ID NO: 29 - RNA, 21 nt, synthetic construct
  source 1..21: mol_type = other RNA
tccatactgagggtgaaatta

SEQ ID NO: 30 - RNA, 21 nt, synthetic construct
  source 1..21: mol_type = other RNA
ccatactgagggtgaaattaa

SEQ ID NO: 31 - RNA, 21 nt, synthetic construct
  source 1..21: mol_type = other RNA
gcagctgaacatatacataga

SEQ ID NO: 32 - RNA, 21 nt, synthetic construct
  source 1..21: mol_type = other RNA
ctgaacatatacatagatgtt

SEQ ID NO: 33 - RNA, 21 nt, synthetic construct
  source 1..21: mol_type = other RNA
gagttgtagttggatttgtct

SEQ ID NO: 34 - RNA, 21 nt, synthetic construct
  source 1..21: mol_type = other RNA
gttgtagttggatttgtctgt

SEQ ID NO: 35 - RNA, 21 nt, synthetic construct
  source 1..21: mol_type = other RNA
ttggatttgtctgtttatgct

SEQ ID NO: 36 - RNA, 21 nt, synthetic construct
  source 1..21: mol_type = other RNA
tggatttgtctgtttatgctt

SEQ ID NO: 37 - RNA, 21 nt, synthetic construct
  source 1..21: mol_type = other RNA
ggatttgtctgtttatgcttt

SEQ ID NO: 38 - RNA, 21 nt, synthetic construct
  source 1..21: mol_type = other RNA
gagtgactatgatagtgaaaa

SEQ ID NO: 39 - RNA, 21 nt, synthetic construct
  source 1..21: mol_type = other RNA
gtgactatgatagtgaaaaga

SEQ ID NO: 40 - RNA, 21 nt, synthetic construct
  source 1..21: mol_type = other RNA
gatagtgaaaagaaaaaaaaa

SEQ ID NO: 41 - RNA, 21 nt, synthetic construct
  source 1..21: mol_type = other RNA
cgcatgtatcttgaaatgctt

SEQ ID NO: 42 - RNA, 21 nt, synthetic construct
  source 1..21: mol_type = other RNA
catgtatcttgaaatgcttgt

SEQ ID NO: 43 - RNA, 21 nt, synthetic construct
  source 1..21: mol_type = other RNA
atcttgaaatgcttgtaaaga

SEQ ID NO: 44 - RNA, 21 nt, synthetic construct
  source 1..21: mol_type = other RNA
gaagcacaggattaggactga

SEQ ID NO: 45 - RNA, 21 nt, synthetic construct
  source 1..21: mol_type = other RNA
gcttacaactcctgcatcaca

SEQ ID NO: 46 - RNA, 21 nt, synthetic construct
  source 1..21: mol_type = other RNA
gagggacttggcagtagaaat

SEQ ID NO: 47 - RNA, 21 nt, synthetic construct
  source 1..21: mol_type = other RNA
gtgggctagataggatatact

SEQ ID NO: 48 - RNA, 21 nt, synthetic construct
  source 1..21: mol_type = other RNA
gggctagataggatatactgt

SEQ ID NO: 49 - RNA, 21 nt, synthetic construct
  source 1..21: mol_type = other RNA
ggctagataggatatactgta

SEQ ID NO: 50 - RNA, 21 nt, synthetic construct
  source 1..21: mol_type = other RNA
gctagataggatatactgtat

SEQ ID NO: 51 - RNA, 21 nt, synthetic construct
  source 1..21: mol_type = other RNA
atcaatagttccatttaaatt

SEQ ID NO: 52 - RNA, 21 nt, synthetic construct
  source 1..21: mol_type = other RNA
caatagttccatttaaattga

SEQ ID NO: 53 - RNA, 21 nt, synthetic construct
  source 1..21: mol_type = other RNA
cccatgagtttgccatgttga

SEQ ID NO: 54 - RNA, 21 nt, synthetic construct
  source 1..21: mol_type = other RNA
cccatgatttcttcggtaatt

SEQ ID NO: 55 - RNA, 21 nt, synthetic construct
  source 1..21: mol_type = other RNA
gtctgtgaatgtctatatagt

SEQ ID NO: 56 - RNA, 21 nt, synthetic construct
  source 1..21: mol_type = other RNA
gtgaatgtctatatagtgtat

SEQ ID NO: 57 - RNA, 21 nt, synthetic construct
  source 1..21: mol_type = other RNA
gtgaatgtctatatagtgtat

SEQ ID NO: 58 - RNA, 21 nt, synthetic construct
  source 1..21: mol_type = other RNA
gtgtattgtgtgttttaacaa

SEQ ID NO: 59 - RNA, 21 nt, synthetic construct
  source 1..21: mol_type = other RNA
gtattgtgtgttttaacaaat

SEQ ID NO: 60 - RNA, 21 nt, synthetic construct
  source 1..21: mol_type = other RNA
ttgtgtgttttaacaaatgat

SEQ ID NO: 61 - RNA, 21 nt, synthetic construct
  source 1..21: mol_type = other RNA
gtgtgttttaacaaatgattt

SEQ ID NO: 62 - RNA, 23 nt, synthetic construct
  source 1..23: mol_type = other RNA
ttccatcacttcgaactcctggc

SEQ ID NO: 63 - RNA, 23 nt, synthetic construct
  source 1..23: mol_type = other RNA
ttggtcttggtgcatggtgtagc

SEQ ID NO: 64 - RNA, 23 nt, synthetic construct
  source 1..23: mol_type = other RNA
attctttcaggccagcgtccgtg

SEQ ID NO: 65 - RNA, 23 nt, synthetic construct
  source 1..23: mol_type = other RNA
tgttggagtgctcttagcatcag

SEQ ID NO: 66 - RNA, 23 nt, synthetic construct
  source 1..23: mol_type = other RNA
taactttggagaggaaatccaca

SEQ ID NO: 67 - RNA, 23 nt, synthetic construct
  source 1..23: mol_type = other RNA
tgatttccacgtgaaacgtgaac

SEQ ID NO: 68 - RNA, 23 nt, synthetic construct
  source 1..23: mol_type = other RNA
ttttgtcatcgcttccagtcctt

SEQ ID NO: 69 - RNA, 23 nt, synthetic construct
  source 1..23: mol_type = other RNA
tattaattatctgcaccttcccg

SEQ ID NO: 70 - RNA, 23 nt, synthetic construct
  source 1..23: mol_type = other RNA
tcttattaattatctgcaccttc

SEQ ID NO: 71 - RNA, 23 nt, synthetic construct
  source 1..23: mol_type = other RNA
tgcttcttattaattatctgcac

SEQ ID NO: 72 - RNA, 23 nt, synthetic construct
  source 1..23: mol_type = other RNA
ttggactggacgttgctaagatc

SEQ ID NO: 73 - RNA, 23 nt, synthetic construct
  source 1..23: mol_type = other RNA
attatcctttgagccacacttgg

SEQ ID NO: 74 - RNA, 23 nt, synthetic construct
  source 1..23: mol_type = other RNA
tttgatattatcctttgagcctt

SEQ ID NO: 75 - RNA, 23 nt, synthetic construct
  source 1..23: mol_type = other RNA
tgtttgatattatcctttgagcc

SEQ ID NO: 76 - RNA, 23 nt, synthetic construct
  source 1..23: mol_type = other RNA
ttgtagactatttgcacactgcc

SEQ ID NO: 77 - RNA, 23 nt, synthetic construct
  source 1..23: mol_type = other RNA
attttacttccacctggccacct

SEQ ID NO: 78 - RNA, 23 nt, synthetic construct
  source 1..23: mol_type = other RNA
tattttacttccacctggccacc

SEQ ID NO: 79 - RNA, 23 nt, synthetic construct
  source 1..23: mol_type = other RNA
tcaatctttttatttcctccgcc

SEQ ID NO: 80 - RNA, 23 nt, synthetic construct
  source 1..23: mol_type = other RNA
tcaatctttttatttcctccttt

SEQ ID NO: 81 - RNA, 23 nt, synthetic construct
  source 1..23: mol_type = other RNA
tttcaatctttttatttcctccg

SEQ ID NO: 82 - RNA, 23 nt, synthetic construct
  source 1..23: mol_type = other RNA
atcacaaaccctgcttggccagg

SEQ ID NO: 83 - RNA, 23 nt, synthetic construct
  source 1..23: mol_type = other RNA
actgattttgaagtcccgagctt

SEQ ID NO: 84 - RNA, 23 nt, synthetic construct
  source 1..23: mol_type = other RNA
aaagaatcaaaaggaattgcctt

SEQ ID NO: 85 - RNA, 23 nt, synthetic construct
  source 1..23: mol_type = other RNA
agtttcaaatcctttgttgcttt

SEQ ID NO: 86 - RNA, 23 nt, synthetic construct
  source 1..23: mol_type = other RNA
aagtttcaaatcctttgttgctt

SEQ ID NO: 87 - RNA, 23 nt, synthetic construct
  source 1..23: mol_type = other RNA
ataaaacagggtttctgtggatt

SEQ ID NO: 88 - RNA, 23 nt, synthetic construct
  source 1..23: mol_type = other RNA
aataaaacagggtttctgtggat

SEQ ID NO: 89 - RNA, 23 nt, synthetic construct
  source 1..23: mol_type = other RNA
aatttcaccctcagtatggagtt

SEQ ID NO: 90 - RNA, 23 nt, synthetic construct
  source 1..23: mol_type = other RNA
taatttcaccctcagtatggatt

SEQ ID NO: 91 - RNA, 23 nt, synthetic construct
  source 1..23: mol_type = other RNA
ttaatttcaccctcagtatggag

SEQ ID NO: 92 - RNA, 23 nt, synthetic construct
  source 1..23: mol_type = other RNA
tctatgtatatgttcagctgctt

SEQ ID NO: 93 - RNA, 23 nt, synthetic construct
  source 1..23: mol_type = other RNA
aacatctatgtatatgttcagtt

SEQ ID NO: 94 - RNA, 23 nt, synthetic construct
  source 1..23: mol_type = other RNA
agacaaatccaactacaactcaa

SEQ ID NO: 95 - RNA, 23 nt, synthetic construct
  source 1..23: mol_type = other RNA
acagacaaatccaactacaactc

SEQ ID NO: 96 - RNA, 23 nt, synthetic construct
  source 1..23: mol_type = other RNA
agcataaacagacaaatccaatt

SEQ ID NO: 97 - RNA, 23 nt, synthetic construct
  source 1..23: mol_type = other RNA
aagcataaacagacaaatccaaa

SEQ ID NO: 98 - RNA, 23 nt, synthetic construct
  source 1..23: mol_type = other RNA
aaagcataaacagacaaatccaa

SEQ ID NO: 99 - RNA, 23 nt, synthetic construct
  source 1..23: mol_type = other RNA
ttttcactatcatagtcactctt

SEQ ID NO: 100 - RNA, 23 nt, synthetic construct
  source 1..23: mol_type = other RNA
tcttttcactatcatagtcactc

SEQ ID NO: 101 - RNA, 23 nt, synthetic construct
  source 1..23: mol_type = other RNA
tttttttttcttttcactatcat

SEQ ID NO: 102 - RNA, 23 nt, synthetic construct
  source 1..23: mol_type = other RNA
aagcatttcaagatacatgcgtt

SEQ ID NO: 103 - RNA, 23 nt, synthetic construct
  source 1..23: mol_type = other RNA
acaagcatttcaagatacatgcg

SEQ ID NO: 104 - RNA, 23 nt, synthetic construct
  source 1..23: mol_type = other RNA
tctttacaagcatttcaagattt

SEQ ID NO: 105 - RNA, 23 nt, synthetic construct
  source 1..23: mol_type = other RNA
tcagtcctaatcctgtgcttctt

SEQ ID NO: 106 - RNA, 23 nt, synthetic construct
  source 1..23: mol_type = other RNA
tgtgatgcaggagttgtaagctt

SEQ ID NO: 107 - RNA, 23 nt, synthetic construct
  source 1..23: mol_type = other RNA
atttctactgccaagtccctctt

SEQ ID NO: 108 - RNA, 23 nt, synthetic construct
  source 1..23: mol_type = other RNA
agtatatcctatctagcccactt

SEQ ID NO: 109 - RNA, 23 nt, synthetic construct
  source 1..23: mol_type = other RNA
acagtatatcctatctagccctt

SEQ ID NO: 110 - RNA, 23 nt, synthetic construct
  source 1..23: mol_type = other RNA
tacagtatatcctatctagcctt

SEQ ID NO: 111 - RNA, 23 nt, synthetic construct
  source 1..23: mol_type = other RNA
atacagtatatcctatctagctt

SEQ ID NO: 112 - RNA, 23 nt, synthetic construct
  source 1..23: mol_type = other RNA
aatttaaatggaactattgattt

SEQ ID NO: 113 - RNA, 23 nt, synthetic construct
  source 1..23: mol_type = other RNA
tcaatttaaatggaactattgat

SEQ ID NO: 114 - RNA, 23 nt, synthetic construct
  source 1..23: mol_type = other RNA
tcaacatggcaaactcatgggtt

SEQ ID NO: 115 - RNA, 23 nt, synthetic construct
  source 1..23: mol_type = other RNA
aattaccgaagaaatcatgggtt

SEQ ID NO: 116 - RNA, 23 nt, synthetic construct
  source 1..23: mol_type = other RNA
actatatagacattcacagactt

SEQ ID NO: 117 - RNA, 23 nt, synthetic construct
  source 1..23: mol_type = other RNA
acactatatagacattcacagac

SEQ ID NO: 118 - RNA, 23 nt, synthetic construct
  source 1..23: mol_type = other RNA
atacactatatagacattcactt

SEQ ID NO: 119 - RNA, 23 nt, synthetic construct
  source 1..23: mol_type = other RNA
ttgttaaaacacacaatacactt

SEQ ID NO: 120 - RNA, 23 nt, synthetic construct
  source 1..23: mol_type = other RNA
atttgttaaaacacacaatacac

SEQ ID NO: 121 - RNA, 23 nt, synthetic construct
  source 1..23: mol_type = other RNA
atcatttgttaaaacacacaatt

SEQ ID NO: 122 - RNA, 23 nt, synthetic construct
  source 1..23: mol_type = other RNA
aaatcatttgttaaaacacacaa

SEQ ID NO: 123 - RNA, 6815 nt, Homo sapiens
  source 1..6815: mol_type = transcribed RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

(19) United States
(12) Patent Application Publication
(10) Pub. No.: US 2026/0167963 A1
(43) Pub. Date: Jun. 18, 2026

(54) Compounds, Compositions and Methods for Reducing MAPT (TAU) Expression

(71) Applicant: SynerK Inc., Grand Cayman (KY)

(72) Inventors: Nan ZHANG, Houston, TX (US); Weiwen JIANG, Winchester, MA (US); Tao LAN, Brookline, MA (US); Dong YU, Westboro, MA (US)

(21) Appl. No.: 19/290,766

(22) Filed: Aug. 5, 2025

Related U.S. Application Data
(60) Provisional application No. 63/688,516, filed on Aug. 29, 2024.

(51) Int. Cl. C12N 15/113; A61P 25/28

(52) U.S. Cl. CPC C12N 15/113; A61P 25/28; C12N 2310/20; C12N 2310/3515

(57) ABSTRACT

The present disclosure relates to the field of nucleic acids, in particular, to compounds, compositions and methods for reducing MAPT (Tau) expression. The nucleic acids of the present disclosure can effectively reduce MAPT (Tau) levels, and they can inhibit MAPT gene expression in cells and animals through the RNA interference mechanism. Meanwhile, compared with other technologies such as antibody therapy, antisense technology and small molecules, RNAi agents can also achieve a lower administration frequency.

### RELATED APPLICATION

This application claims the benefit of U.S. Provisional Application No. 63/688,516, filed on Aug. 29, 2024. The entire teachings of the above application are incorporated herein by reference.

### SEQUENCE LISTING

The sequence listing submitted via EFS, in compliance with 37 CFR § 1.52(e)(5), is incorporated herein by reference. The sequence listing XML file submitted via EFS contains the file “42693009US1SEQListing.xml”, created on Jul. 30, 2025, which is 116,301 bytes in size.

### TECHNICAL FIELD

The present disclosure relates to the field of nucleic acids, in particular, to compounds, pharmaceutical compositions and methods that reduce the quantity or activity of Tau mRNA and that in certain instances reduce Tau protein and/or aggregate level. Such Tau-lowering interventions are beneficial to ameliorating symptoms of a range of neurodegenerative and neurological disorders, including but not limited to Alzheimer's disease (AD), frontotemporal dementia (FTD), progressive supranuclear palsy (PSP), corticobasal ganglionic degeneration (CBD), Pickle's disease (PiD), agranulocytosis granulomatosa (AGD), subacute sclerosing proptosis total encephalitis (SSPE), Christiansen's syndrome (CS), post-encephalitis Parkinson's syndrome (PEP), Guadeloupe Parkinson's syndrome (GP), globoid glial cell tauopathy (GGT), spinal cerebellar ataxia type 11 (SCA11), chronic traumatic encephalopathy (CTE), aging-associated tau astrocytosis (ARTAG), and primary age-related tauopathy (PART).

### BACKGROUND

The microtubule associated protein Tau (MAPT) gene is located in the human chromosomal region 17q21.31 and is predominantly expressed in the central and peripheral nervous system (CNS and PNS respectively). At a molecular level, Tau interacts with tubulin to stabilize microtubules—a critical component of the cytoskeleton. Mutations and hyperphosphorylations of Tau cause its dissociation from microtubules and subsequent self-aggregation and deposition in neurofibrillary tangles (NFTs), leading to neurodegenerative tauopathies such as AD, FTD and amongst others. Aberrant Tau also contributes to pathogenesis of Down syndrome, epilepsy, depression, Huntington's disease (HD) and Parkinson's disease (PD). Currently, there are no effective interventions to tauopathies but palliative care. Although two FDA-approved, amyloid beta (Aβ)-targeting antibodies, namely aducanumab and lecanemab, concomitantly lower the level of hyperphosphorylated Tau in AD patients, they both come with class side-effects such as brain swelling and microbleeding. Clearly, it calls for an alternative Tau-lowering therapeutic modality, such as by employing RNA interference (RNAi).

Tau is a well-established microtubule binding protein in neurons. Its coding gene, MAPT, comprises of 16 exons and is differentially spliced and developmentally regulated. Exons 9-12 each encode an imperfect repeat (R1-R4 respectively) responsible for microtubule binding, and along with two alternatively spliced N-terminal exons (N1 and N2) and four constitutively transcribed exons, they form six Tau isoforms (0N3R, 1N3R, 2N3R, 0N4R, 1N4R and 2N4R). All six isoforms are expressed in normal adult human brain, and the 4R/3R isoform ratio is approximately equal to 1. This ratio is frequently distorted across neurodegenerative diseases, indicating heterogeneity in the composition of Tau aggregates and their related clinical manifestations. For instance, AD, Down syndrome and CTE brains display Tau aggregates with a mixed 4R/3R ratio. 4R isoforms are predominantly present in PSP, CBD, HD, AGD and GGT brains, while 3R species are mainly found in FTD and PiD brains. In adult murine brain, 4R isoforms are almost exclusively expressed. With more microtubule binding repeats present, 4R isoforms assemble into aggregates 2.5-3 times faster than their 3R counterparts.

Tau contains 85 putative phosphorylation sites, including 45 serines, 35 threonines and 5 tyrosines. Hyperphosphorylation at these sites has profound impacts on neuronal survival and synaptic function via several pathomechanisms: (1) it destabilizes the neuronal cytoskeleton network; (2) hyperphosphorylated Tau interferes with cargo loading of motor proteins and their access to (and distance along) the microtubule tracks; (3) hyperphosphorylated Tau mislocalizes from the axonal compartment to the somatodendritic compartment, causing perturbation of AMPA (α-amino-3-hydroxy-5-methyl-4-isoxazolepropionic acid) and NMDA (N-methyl-d-aspartate) signaling, excitotoxicity and synaptic dysfunction; (4) an increase in dendritic Tau causes missorting of mitochondria, vesicles and neurofilaments in affected neurons; and (5) hyperphosphorylated Tau is recruited to stress granules, sensitizing neurons to cytotoxic stress. In addition to phosphorylation, other forms of post-translational modification, such as acetylation, proteolysis, glycosylation, ubiquitination and SUMOylation, further modulate Tau's property and cellular function.

The most prevalent pathogenesis of tauopathy is underpinned by misfolded Tau oligomerizing to insoluble deposits that gradually overburden neurons to cell death. Hyperphosphorylated Tau self-associates to form a myriad of conformational “strains”, such as small oligomers, paired helical fragments (PHFs), straight filaments, neuropil threads, fibrillar aggregates and NFTs. The propensity of Tau to self-aggregate lies in two hexapeptides within the microtubule binding motifs (PHF6* in R2 and PHF6 in R3). Dimerization of Tau at PHF6s initiates a nucleation site, and once a critical cluster size is reached, Tau oligomerization proceeds in a dose- and time-dependent manner. Although it is still unclear as to which of these many Tau “strains” contribute to the neuropathological heterogeneity observed across tauopathies, accumulating evidence suggests that soluble Tau oligomers generated during NFT formation are most toxic to neurons and synapses (at least in AD) and that they can propagate trans-syntactically in a prion-like fashion. Interestingly, intraperitoneal injection of brain homogenates containing Tau seeds gives rise to cerebral Tau deposition in transgenic mice, suggesting that peripheral Tau aggregates could modify brain tauopathy.

AD is a neurodegenerative tauopathy known to be the most common form of dementia. It affects approximately 55 million people worldwide and has an annual healthcare burden of over $355 billion in the US in 2021. Although extracellular amyloid beta (Aβ) plaques and intracellular Tau NFTs are cardinal features of AD, Tau burden correlates more closely with cognitive decline than does Aβ burden, and the removal of Tau burden significantly improves cognitive function even when Aβ burden remained constant or increased. Based on the Braak spatiotemporal progression pattern, Tau NFT initiates in the transentorhinal region, subsequently spreads to the hippocampus, limbic structures and isocortical areas, and finally reaches the neocortex and other parts of the AD brain. In addition to neuroanatomical connection between these brain parts, the dissemination of Tau is also facilitated by activated microglia via phagocytosis and exosomal secretion, as suggested by recent transgenic animal data and positron emission topography (PET) studies on AD patients. More than 40 potential phosphorylation sites of the Tau protein have been identified as targets of glycogen synthase kinase 3 (GSK3), among which over 29 sites are phosphorylated in the brains of AD patients. The activity of GSK3 is positively correlated with Tau burden in AD patients. Inhibition of GSK3 can reduce Tau phosphorylation, axonal degeneration, and neuronal loss, showing positive effects in transgenic AD mice. However, clinical trials of GSK3 inhibitors have not yet achieved positive primary results.

Several studies suggest that Tau ablation does not raise serious safety concerns for treating tauopathies. For instance, Tau^{-/-} knockout mice are physically normal and able to reproduce. In aged Tau^{-/-} and Tau^{-/-} mice, Tau ablation did not impair cognition, but confers excitoprotective effects against seizure and only causes minor motor deficits that were much more subtle than those associated with aging. Moreover, Tau ablation prevents synaptic and cognitive dysfunction in several transgenic AD mouse models.

In a recent phase 1b clinical trial using antisense oligonucleotide (ASO) against Tau mRNA, patients with mild AD exhibited a decent safety profile and sustained reduction of total-Tau (t-Tau) and t-Tau/Aβ₄₂ ratio (by 50% and 40% respectively) in the cerebrospinal fluid (CSF). These positive outcomes were achieved at the highest tested dose (115 mg injected every 3 months) 197 days after the initial intrathecal injection.

Tau monomers form fibrils and then aggregates into intracellular NFTs, but they are also secreted in a prion-like manner to fuel transcellular seeding. The most studied Tau-lowering antibody, namely gosuranemab/BIIB092, targets an amino terminal region of the extracellular Tau (eTau) isolated from familial AD patient cells. Although proven clinically safe and able to reduce the amount of unbound eTau by >90% in patient CSF, this antibody failed to show efficacy on primary, secondary, or exploratory outcomes in both PSP and AD tauopathies. It is likely that (1) gosuranemab does not disrupt the blood-brain barrier (BBB) to reach the brain parenchyma, and (2) its modality as an antibody does not permit efficient entry into neurons for intracellular tangle elimination. As a consequence, although gosuranemab showed a dose-dependent CSF accumulation, possibly through the leaky choroid plexus, it had no effects on phosphorylated Tau levels or Tau inclusions in the brain.

Thus, there is an urgent need to have novel therapies to target Tau.

RNA interference (RNAi) is a highly conserved regulatory mechanism that can downregulate gene expression through the delivery of synthetic small interfering RNA (siRNA) to cells or animals. Unlike antisense oligonucleotides (ASOs) that degrade RNA using RNase H, siRNA exerts its function through the RNA-induced silencing complex (RISC) in the cytoplasm. siRNA has a high degree of reprogrammability for different target sequences and can undergo various chemical modifications and conjugations. As of August 2023, five siRNA drugs have been approved by the FDA for marketing. These siRNA drugs have shown excellent safety, long-lasting therapeutic effects (some can be injected once every six months), and require lower doses than ASOs. After conjugation with 2′-O-hexadecyl (C16), the candidate siRNA can reduce the APP mRNA level in the central nervous system by approximately 75% at 129 days after a single 60 mg intrathecal injection in non-human primates.

### SUMMARY OF THE INVENTION

The present disclosure aims to overcome the problems in the prior art and provide a novel nucleic acid targeting MAPT (Tau) and uses thereof. Preferably, the nucleic acid targeting Tau is a synthetic small interfering RNA (siRNA).

The present disclosure first provides a nucleic acid, which comprises a sense strand and an antisense strand, wherein the sense strand contains a sequence having more than 80% sequence identity to any of the sequences shown in SEQ ID NO: 1-61, and the antisense strand contains a sequence having more than 80% sequence identity to any of the sequences shown in SEQ ID NO: 62-122.

The present disclosure further provides a targeted drug delivery system, which comprises a targeting moiety, a linking moiety, and any of the aforementioned nucleic acids linked to the targeting moiety via the linking moiety.

The present disclosure further provides an isolated cell containing any of the aforementioned nucleic acids or any of the aforementioned targeted drug delivery systems.

The present disclosure also provides a pharmaceutical composition, which contains any of the aforementioned nucleic acids or any of the aforementioned targeted drug delivery systems, and a pharmaceutically acceptable carrier.

The present disclosure further provides a method of inhibiting MAPT expression in a cell, which comprises: contacting the cell with any of the aforementioned nucleic acids, any of the aforementioned targeted drug delivery systems, or the aforementioned pharmaceutical composition to inhibit MAPT expression in the cell.

The present disclosure also provides the use of any of the aforementioned nucleic acids, any of the aforementioned targeted drug delivery systems, or the aforementioned pharmaceutical composition in any of the following aspects: 1) treating and/or preventing MAPT-related diseases; 2) preparing a medicament for treating and/or preventing MAPT-related diseases.

The present disclosure further provides a method of using any of the aforementioned nucleic acids, any of the aforementioned targeted drug delivery systems, or the aforementioned pharmaceutical composition in any of the following aspects: 1) treating and/or preventing MAPT-related diseases; 2) preparing medicament for treating and/or preventing MAPT-related diseases.

The nucleic acid of the present disclosure can effectively reduce the level of MAPT (Tau) and can inhibit MAPT gene expression in cells and animals through the RNA interference mechanism. Meanwhile, compared with other technologies such as antibody therapy, antisense technology, and small molecules, RNAi agents can achieve a lower dosing frequency.

### BRIEF DESCRIPTION OF THE DRAWINGS

To more clearly illustrate the specific embodiments of the present disclosure or the technical solutions in the prior art, the accompanying drawings required for describing the specific embodiments or the prior art will be briefly introduced below. Apparently, the drawings in the following description are some embodiments of the present disclosure. For those of ordinary skill in the art, other drawings can also be obtained based on these drawings without exerting inventive work.

FIG. 1 is the in vitro dose-response curve of SN-5182 and SN-5196 on endogenous MAPT mRNA 24 hours after electroporation in SH-SY5Y cells in the examples of the present disclosure.

FIG. 2 is the in vitro dose-response curve of SN-5182 and SN-5196 on endogenous MAPT mRNA 48 hours after transfection in Hep3B cells in the examples of the present disclosure.

### DETAILED DESCRIPTION

The specific embodiments of the present disclosure are described in detail below. It should be understood that the specific embodiments described herein are only used to illustrate and explain the present disclosure, and are not intended to limit the present disclosure. Those skilled in the art can make various modifications and changes to the present disclosure without departing from the scope or spirit thereof. For example, features illustrated or described as part of one embodiment can be used in another embodiment to yield a still further embodiment.

### Definition of Terms

Unless otherwise specified, all terms (including technical and scientific terms) used to disclose the present disclosure have the same meaning as commonly understood by those of ordinary skill in the art to which the present disclosure belongs. By way of further guidance, the following definitions are provided to better understand the teachings of the present disclosure. The terms used in the description of the present disclosure are only for the purpose of describing specific embodiments and are not intended to limit the present disclosure.

The terms “and/or” and “or/and” as used herein include any one of two or more related listed items, as well as any and all combinations of the related listed items. Such any and all combinations include: any combination of any two of the listed items, any combination of more than two of the listed items, and a combination of all the listed items. It should be noted that when at least three items are connected by at least two conjunctions selected from “and/or” and “or/and”, it should be understood that in this application, the technical solution undoubtedly includes the technical solution connected by “logical AND” throughout, and also undoubtedly includes the technical solution connected by “logical OR” throughout. For example, “A and/or B” includes three parallel solutions: A, B, and A+B. Another example is that the technical solution of “A, and/or, B, and/or, C, and/or, D” includes any one of A, B, C, and D (i.e., the technical solution connected by “logical OR” throughout), and also includes any and all combinations of A, B, C, and D, that is, combinations of any two or any three of A, B, C, and D, as well as the combination of A, B, C, and D (i.e., the technical solution connected by “logical AND” throughout).

The terms “comprising”, “comprises”, “comprise”; “including”, “includes”, “include”; and “containing”, “contains”, “contain” as used herein are synonyms, which are inclusive or open-ended and do not exclude additional, unrecited members, elements, or method steps.

In the present disclosure, numerical ranges expressed by endpoints include all numerical values and fractions contained within the range, as well as the cited endpoints.

In the present disclosure, when referring to concentration values, it means that fluctuations within a certain range are included. For example, fluctuations within the corresponding precision range are allowed. For example, 2% may allow a fluctuation within ±0.1%. For larger values or values that do not require overly precise control, larger fluctuations are also allowed. For example, 80% may allow fluctuations within ±1%, ±2%, ±5%, etc.

In the present disclosure, descriptions involving “a plurality of” or “multiple” refer to a quantity of 2 or more, unless otherwise specified.

In the present disclosure, among the technical features described in an open manner, they include closed technical solutions consisting of the listed features, as well as open technical solutions containing the listed features.

In the present disclosure, the terms “preferably”, “more preferably”, and “even more preferably” are only used to describe embodiments or examples with better effects, and it should be understood that they do not constitute a limitation on the protection scope of the present disclosure.

In the present disclosure, the terms “optionally” and “optional” mean that the corresponding element or step may be “present” or “absent”. If multiple instances of “optional” or “optionally” appear in a technical solution, unless otherwise specified and in the absence of contradictions or mutual restrictions, each instance of “optional” or “optionally” is independent of the others.

In the present disclosure, the term “nucleic acid” refers to a composition containing RNA or RNA-like (e.g., chemically modified RNA) oligonucleotide molecules that can degrade or inhibit (e.g., degrade or inhibit under appropriate conditions) the translation of messenger RNA (mRNA) transcripts of target mRNA in a sequence-specific manner. The nucleic acid can function through the RNA interference mechanism (i.e., inducing RNA interference by interacting with the RNA interference pathway mechanism (RNA-induced silencing complex or RISC) of mammalian cells) or through any alternative mechanism or pathway. The defined scope of nucleic acids including sense and antisense strands disclosed herein includes, but is not limited to: short (or small) interfering RNA (siRNA), double-stranded RNA (dsRNA), microRNA (miRNA), short hairpin RNA (shRNA), and dicer substrates.

In the present disclosure, when it is mentioned that “the antisense strand (sense strand) contains at least 15 contiguous nucleotides, and the at least 15 contiguous nucleotides differ by no more than 3 nucleotides from any at least 15 contiguous nucleotides in the reference sequence (such as the sequence shown in SEQ ID NO: X or the nucleotide sequence at positions 1-21 of the shown sequence)”, the involved alignment situations include alignment with at least 15 contiguous nucleotides starting at any position in the reference sequence (such as positions 1, 2, 3, . . . , 7, or 8). As an example, in some embodiments, the antisense strand (sense strand) contains 21 contiguous nucleotides, where the nucleotide sequence at positions 1-15 has 1, 2, or 3 differences from the nucleotide sequence at positions 2-16 of the reference sequence, and the nucleotides at positions 16-20 in the antisense strand (sense strand) are all the same as or all different from the nucleotide sequence at positions 17-21 of the reference sequence; such sequences are also within the defined scope of the present disclosure. In some embodiments, the antisense strand (sense strand) contains 21 contiguous nucleotides, where the nucleotide sequence at positions 1-15 is identical to the nucleotide sequence at positions 1-15 of the reference sequence, and the nucleotides at positions 16-21 in the antisense strand (sense strand) are all the same as or all different from the nucleotide sequence at positions 16-21 of the reference sequence; such sequences are also within the defined scope of the present disclosure.

In the present disclosure, when referring to descriptions related to sequence alignment, the mentioned “difference” or “differ from” includes one or more of substitution, insertion, and deletion.

In the present disclosure, when referring to the expression of a given gene, the terms “silence”, “reduce”, “inhibit”, “down-regulate”, or “knockdown” mean that the expression of the gene is reduced when a cell, cell population, tissue, organ, or subject is treated with the nucleic acid described herein, as measured by the level of RNA transcribed by the gene or the level of polypeptide, protein, or protein subunit translated from mRNA in the cell, cell population, tissue, organ, or subject where the gene is transcribed, compared to a second untreated cell, cell population, tissue, organ, or subject.

In the present disclosure, “fully complementary” means that in a hybrid pair of nucleobase or nucleotide sequence molecules, all (100%) bases in the contiguous sequence of the first oligonucleotide hybridize with the same number of bases in the contiguous sequence of the second oligonucleotide. The contiguous sequence may include all or part of the first nucleotide sequence or the second nucleotide sequence.

In the present disclosure, “partially complementary” means that in a hybrid pair of nucleobase or nucleotide sequence molecules, at least 70% but not all of the bases in the contiguous sequence of the first oligonucleotide hybridize with the same number of bases in the contiguous sequence of the second oligonucleotide. The contiguous sequence may include all or part of the first nucleotide sequence or the second nucleotide sequence.

In the present disclosure, “substantially complementary” means that in a hybrid pair of nucleobase or nucleotide sequence molecules, at least 85% but not all of the bases in the contiguous sequence of the first oligonucleotide hybridize with the same number of bases in the contiguous sequence of the second oligonucleotide. The contiguous sequence may include all or part of the first nucleotide sequence or the second nucleotide sequence.

In the present disclosure, when referring to “at least partially complementary”, it means that in a hybrid pair of nucleobase or nucleotide sequence molecules, the first oligonucleotide and the second oligonucleotide are partially complementary, substantially complementary, or fully complementary.

In the present disclosure, the term “treatment” refers to a method or step taken to provide relief or alleviation of the number, severity, and/or frequency of one or more disease symptoms in a subject. The treatment may include prevention, management, prophylactic treatment, and/or inhibition or reduction of the number, severity, and/or frequency of one or more disease symptoms in the subject.

In the present disclosure, the term “link”, “linking” or “linked” means that two compounds or molecules are joined by a covalent bond. Unless otherwise specified, as used herein, the term “link”, “linking” or “linked” may refer to a connection between a first compound and a second compound with or without any intermediate atoms or groups of atoms.

In the present disclosure, the term “nucleotide containing an unnatural base” refers to the replacement of natural bases (adenine, uracil, guanine, cytosine) in RNA molecules with unnatural bases through chemical synthesis. The introduction of these unnatural bases is intended to optimize the performance of RNAi agents, such as enhancing stability, improving specificity, reducing immunogenicity, or endowing new functions.

### Nucleic Acid

The present disclosure first provides a (modified or unmodified) nucleic acid, which comprises a sense strand and an antisense strand. The sense strand contains a sequence having more than 80% (e.g., 80%, 81%, 82%, 83%, 84%, 85%, 86%, 87%, 88%, 89%, 90%, 91%, 92%, 93%, 94%, 95%, 96%, 97%, 98%, 99%) sequence identity to any of the sequences shown in SEQ ID NO: 1-61. The antisense strand contains a sequence having more than 80% (e.g., 80%, 81%, 82%, 83%, 84%, 85%, 86%, 87%, 88%, 89%, 90%, 91%, 92%, 93%, 94%, 95%, 96%, 97%, 98%, 99%) sequence identity to any of the sequences shown in SEQ ID NO: 62-122.

In some embodiments, the antisense strand has 15-30 (e.g., 15, 16, 17, 18, 19, 20, 21, 22, 23, 24, 25, 26, 27, 28, 29, or 30) nucleotides (bases).

In some embodiments, the sense strand has 15-30 (e.g., 15, 16, 17, 18, 19, 20, 21, 22, 23, 24, 25, 26, 27, 28, 29, or 30) nucleotides (bases).

In the present disclosure, the sense strand and the antisense strand may have the same length or different lengths.

In specific embodiments, those skilled in the art can combine the sequences provided in the present disclosure by considering the complementarity of the sense strand and the antisense strand, thereby obtaining the combined nucleic acid (such as siRNA).

In a preferred embodiment of the present disclosure, as shown in Table 1, the nucleic acid is selected from at least one of the following: siRNA-1, with the sense strand sequence being SEQ ID NO: 1 and the antisense strand sequence being SEQ ID NO: 62; siRNA-2, with the sense strand sequence being SEQ ID NO: 2 and the antisense strand sequence being SEQ ID NO: 63; siRNA-3, with the sense strand sequence being SEQ ID NO: 3 and the antisense strand sequence being SEQ ID NO: 64; siRNA-4, with the sense strand sequence being SEQ ID NO: 4 and the antisense strand sequence being SEQ ID NO: 65; siRNA-5, with the sense strand sequence being SEQ ID NO: 5 and the antisense strand sequence being SEQ ID NO: 66; siRNA-6, with the sense strand sequence being SEQ ID NO: 6 and the antisense strand sequence being SEQ ID NO: 67; siRNA-7, with the sense strand sequence being SEQ ID NO: 7 and the antisense strand sequence being SEQ ID NO: 68; siRNA-8, with the sense strand sequence being SEQ ID NO: 8 and the antisense strand sequence being SEQ ID NO: 69; siRNA-9, with the sense strand sequence being SEQ ID NO: 9 and the antisense strand sequence being SEQ ID NO: 70; siRNA-10, with the sense strand sequence being SEQ ID NO: 10 and the antisense strand sequence being SEQ ID NO: 71; siRNA-11, with the sense strand sequence being SEQ ID NO: 11 and the antisense strand sequence being SEQ ID NO: 72; siRNA-12, with the sense strand sequence being SEQ ID NO: 12 and the antisense strand sequence being SEQ ID NO: 73; siRNA-13, with the sense strand sequence being SEQ ID NO: 13 and the antisense strand sequence being SEQ ID NO: 74; siRNA-14, with the sense strand sequence being SEQ ID NO: 14 and the antisense strand sequence being SEQ ID NO: 75; siRNA-15, with the sense strand sequence being SEQ ID NO: 15 and the antisense strand sequence being SEQ ID NO: 76; siRNA-16, with the sense strand sequence being SEQ ID NO: 16 and the antisense strand sequence being SEQ ID NO: 77; siRNA-17, with the sense strand sequence being SEQ ID NO: 17 and the antisense strand sequence being SEQ ID NO: 78; siRNA-18, with the sense strand sequence being SEQ ID NO: 18 and the antisense strand sequence being SEQ ID NO: 79; siRNA-19, with the sense strand sequence being SEQ ID NO: 19 and the antisense strand sequence being SEQ ID NO: 80; siRNA-20, with the sense strand sequence being SEQ ID NO: 20 and the antisense strand sequence being SEQ ID NO: 81; siRNA-21, with the sense strand sequence being SEQ ID NO: 21 and the antisense strand sequence being SEQ ID NO: 82; siRNA-22, with the sense strand sequence being SEQ ID NO: 22 and the antisense strand sequence being SEQ ID NO: 83; siRNA-23, with the sense strand sequence being SEQ ID NO: 23 and the antisense strand sequence being SEQ ID NO: 84; siRNA-24, with the sense strand sequence being SEQ ID NO: 24 and the antisense strand sequence being SEQ ID NO: 85; siRNA-25, with the sense strand sequence being SEQ ID NO: 25 and the antisense strand sequence being SEQ ID NO: 86; siRNA-26, with the sense strand sequence being SEQ ID NO: 26 and the antisense strand sequence being SEQ ID NO: 87; siRNA-27, with the sense strand sequence being SEQ ID NO: 27 and the antisense strand sequence being SEQ ID NO: 88; siRNA-28, with the sense strand sequence being SEQ ID NO: 28 and the antisense strand sequence being SEQ ID NO: 89; siRNA-29, with the sense strand sequence being SEQ ID NO: 29 and the antisense strand sequence being SEQ ID NO: 90; siRNA-30, with the sense strand sequence being SEQ ID NO: 30 and the antisense strand sequence being SEQ ID NO: 91; siRNA-31, with the sense strand sequence being SEQ ID NO: 31 and the antisense strand sequence being SEQ ID NO: 92; siRNA-32, with the sense strand sequence being SEQ ID NO: 32 and the antisense strand sequence being SEQ ID NO: 93; siRNA-33, with the sense strand sequence being SEQ ID NO: 33 and the antisense strand sequence being SEQ ID NO: 94; siRNA-34, with the sense strand sequence being SEQ ID NO: 34 and the antisense strand sequence being SEQ ID NO: 95; siRNA-35, with the sense strand sequence being SEQ ID NO: 35 and the antisense strand sequence being SEQ ID NO: 96; siRNA-36, with the sense strand sequence being SEQ ID NO: 36 and the antisense strand sequence being SEQ ID NO: 97; siRNA-37, with the sense strand sequence being SEQ ID NO: 37 and the antisense strand sequence being SEQ ID NO: 98; siRNA-38, with the sense strand sequence being SEQ ID NO: 38 and the antisense strand sequence being SEQ ID NO: 99; siRNA-39, with the sense strand sequence being SEQ ID NO: 39 and the antisense strand sequence being SEQ ID NO: 100; siRNA-40, with the sense strand sequence being SEQ ID NO: 40 and the antisense strand sequence being SEQ ID NO: 101; siRNA-41, with the sense strand sequence being SEQ ID NO: 41 and the antisense strand sequence being SEQ ID NO: 102; siRNA-42, with the sense strand sequence being SEQ ID NO: 42 and the antisense strand sequence being SEQ ID NO: 103; siRNA-43, with the sense strand sequence being SEQ ID NO: 43 and the antisense strand sequence being SEQ ID NO: 104; siRNA-44, with the sense strand sequence being SEQ ID NO: 44 and the antisense strand sequence being SEQ ID NO: 105; siRNA-45, with the sense strand sequence being SEQ ID NO: 45 and the antisense strand sequence being SEQ ID NO: 106; siRNA-46, with the sense strand sequence being SEQ ID NO: 46 and the antisense strand sequence being SEQ ID NO: 107; siRNA-47, with the sense strand sequence being SEQ ID NO: 47 and the antisense strand sequence being SEQ ID NO: 108; siRNA-48, with the sense strand sequence being SEQ ID NO: 48 and the antisense strand sequence being SEQ ID NO: 109; siRNA-49, with the sense strand sequence being SEQ ID NO: 49 and the antisense strand sequence being SEQ ID NO: 110; siRNA-50, with the sense strand sequence being SEQ ID NO: 50 and the antisense strand sequence being SEQ ID NO: 111; siRNA-51, with the sense strand sequence being SEQ ID NO: 51 and the antisense strand sequence being SEQ ID NO: 112; siRNA-52, with the sense strand sequence being SEQ ID NO: 52 and the antisense strand sequence being SEQ ID NO: 113; siRNA-53, with the sense strand sequence being SEQ ID NO: 53 and the antisense strand sequence being SEQ ID NO: 114; siRNA-54, with the sense strand sequence being SEQ ID NO: 54 and the antisense strand sequence being SEQ ID NO: 115; siRNA-55, with the sense strand sequence being SEQ ID NO: 55 and the antisense strand sequence being SEQ ID NO: 116; siRNA-56, with the sense strand sequence being SEQ ID NO: 56 and the antisense strand sequence being SEQ ID NO: 117; siRNA-57, with the sense strand sequence being SEQ ID NO: 57 and the antisense strand sequence being SEQ ID NO: 118; siRNA-58, with the sense strand sequence being SEQ ID NO: 58 and the antisense strand sequence being SEQ ID NO: 119; siRNA-59, with the sense strand sequence being SEQ ID NO: 59 and the antisense strand sequence being SEQ ID NO: 120; siRNA-60, with the sense strand sequence being SEQ ID NO: 60 and the antisense strand sequence being SEQ ID NO: 121; siRNA-61, with the sense strand sequence being SEQ ID NO: 61 and the antisense strand sequence being SEQ ID NO: 122.

In a preferred embodiment of the present disclosure, the nucleic acid is one of the following: siRNA-1, with the sense strand sequence being SEQ ID NO: 1 and the antisense strand sequence being SEQ ID NO: 62; siRNA-2, with the sense strand sequence being SEQ ID NO: 2 and the antisense strand sequence being SEQ ID NO: 63; siRNA-3, with the sense strand sequence being SEQ ID NO: 3 and the antisense strand sequence being SEQ ID NO: 64; siRNA-4, with the sense strand sequence being SEQ ID NO: 4 and the antisense strand sequence being SEQ ID NO: 65; siRNA-5, with the sense strand sequence being SEQ ID NO: 5 and the antisense strand sequence being SEQ ID NO: 66; siRNA-6, with the sense strand sequence being SEQ ID NO: 6 and the antisense strand sequence being SEQ ID NO: 67; siRNA-7, with the sense strand sequence being SEQ ID NO: 7 and the antisense strand sequence being SEQ ID NO: 68; siRNA-8, with the sense strand sequence being SEQ ID NO: 8 and the antisense strand sequence being SEQ ID NO: 69; siRNA-9, with the sense strand sequence being SEQ ID NO: 9 and the antisense strand sequence being SEQ ID NO: 70; siRNA-10, with the sense strand sequence being SEQ ID NO: 10 and the antisense strand sequence being SEQ ID NO: 71; siRNA-11, with the sense strand sequence being SEQ ID NO: 11 and the antisense strand sequence being SEQ ID NO: 72; siRNA-12, with the sense strand sequence being SEQ ID NO: 12 and the antisense strand sequence being SEQ ID NO: 73; siRNA-13, with the sense strand sequence being SEQ ID NO: 13 and the antisense strand sequence being SEQ ID NO: 74; siRNA-14, with the sense strand sequence being SEQ ID NO: 14 and the antisense strand sequence being SEQ ID NO: 75; siRNA-15, with the sense strand sequence being SEQ ID NO: 15 and the antisense strand sequence being SEQ ID NO: 76; siRNA-16, with the sense strand sequence being SEQ ID NO: 16 and the antisense strand sequence being SEQ ID NO: 77; siRNA-17, with the sense strand sequence being SEQ ID NO: 17 and the antisense strand sequence being SEQ ID NO: 78; siRNA-18, with the sense strand sequence being SEQ ID NO: 18 and the antisense strand sequence being SEQ ID NO: 79; siRNA-19, with the sense strand sequence being SEQ ID NO: 19 and the antisense strand sequence being SEQ ID NO: 80; siRNA-20, with the sense strand sequence being SEQ ID NO: 20 and the antisense strand sequence being SEQ ID NO: 81; siRNA-21, with the sense strand sequence being SEQ ID NO: 21 and the antisense strand sequence being SEQ ID NO: 82; siRNA-22, with the sense strand sequence being SEQ ID NO: 22 and the antisense strand sequence being SEQ ID NO: 83; siRNA-23, with the sense strand sequence being SEQ ID NO: 23 and the antisense strand sequence being SEQ ID NO: 84; siRNA-24, with the sense strand sequence being SEQ ID NO: 24 and the antisense strand sequence being SEQ ID NO: 85; siRNA-25, with the sense strand sequence being SEQ ID NO: 25 and the antisense strand sequence being SEQ ID NO: 86; siRNA-26, with the sense strand sequence being SEQ ID NO: 26 and the antisense strand sequence being SEQ ID NO: 87; siRNA-27, with the sense strand sequence being SEQ ID NO: 27 and the antisense strand sequence being SEQ ID NO: 88; siRNA-28, with the sense strand sequence being SEQ ID NO: 28 and the antisense strand sequence being SEQ ID NO: 89; siRNA-29, with the sense strand sequence being SEQ ID NO: 29 and the antisense strand sequence being SEQ ID NO: 90; siRNA-30, with the sense strand sequence being SEQ ID NO: 30 and the antisense strand sequence being SEQ ID NO: 91; siRNA-31, with the sense strand sequence being SEQ ID NO: 31 and the antisense strand sequence being SEQ ID NO: 92; siRNA-32, with the sense strand sequence being SEQ ID NO: 32 and the antisense strand sequence being SEQ ID NO: 93; siRNA-33, with the sense strand sequence being SEQ ID NO: 33 and the antisense strand sequence being SEQ ID NO: 94; siRNA-34, with the sense strand sequence being SEQ ID NO: 34 and the antisense strand sequence being SEQ ID NO: 95; siRNA-35, with the sense strand sequence being SEQ ID NO: 35 and the antisense strand sequence being SEQ ID NO: 96; siRNA-36, with the sense strand sequence being SEQ ID NO: 36 and the antisense strand sequence being SEQ ID NO: 97; siRNA-37, with the sense strand sequence being SEQ ID NO: 37 and the antisense strand sequence being SEQ ID NO: 98; siRNA-38, with the sense strand sequence being SEQ ID NO: 38 and the antisense strand sequence being SEQ ID NO: 99; siRNA-39, with the sense strand sequence being SEQ ID NO: 39 and the antisense strand sequence being SEQ ID NO: 100; siRNA-40, with the sense strand sequence being SEQ ID NO: 40 and the antisense strand sequence being SEQ ID NO: 101; siRNA-41, with the sense strand sequence being SEQ ID NO: 41 and the antisense strand sequence being SEQ ID NO: 102; siRNA-42, with the sense strand sequence being SEQ ID NO: 42 and the antisense strand sequence being SEQ ID NO: 103; siRNA-43, with the sense strand sequence being SEQ ID NO: 43 and the antisense strand sequence being SEQ ID NO: 104; siRNA-44, with the sense strand sequence being SEQ ID NO: 44 and the antisense strand sequence being SEQ ID NO: 105; siRNA-45, with the sense strand sequence being SEQ ID NO: 45 and the antisense strand sequence being SEQ ID NO: 106; siRNA-46, with the sense strand sequence being SEQ ID NO: 46 and the antisense strand sequence being SEQ ID NO: 107; siRNA-47, with the sense strand sequence being SEQ ID NO: 47 and the antisense strand sequence being SEQ ID NO: 108; siRNA-48, with the sense strand sequence being SEQ ID NO: 48 and the antisense strand sequence being SEQ ID NO: 109; siRNA-49, with the sense strand sequence being SEQ ID NO: 49 and the antisense strand sequence being SEQ ID NO: 110; siRNA-50, with the sense strand sequence being SEQ ID NO: 50 and the antisense strand sequence being SEQ ID NO: 111; siRNA-51, with the sense strand sequence being SEQ ID NO: 51 and the antisense strand sequence being SEQ ID NO: 112; siRNA-52, with the sense strand sequence being SEQ ID NO: 52 and the antisense strand sequence being SEQ ID NO: 113; siRNA-53, with the sense strand sequence being SEQ ID NO: 53 and the antisense strand sequence being SEQ ID NO: 114; siRNA-54, with the sense strand sequence being SEQ ID NO: 54 and the antisense strand sequence being SEQ ID NO: 115; siRNA-55, with the sense strand sequence being SEQ ID NO: 55 and the antisense strand sequence being SEQ ID NO: 116; siRNA-56, with the sense strand sequence being SEQ ID NO: 56 and the antisense strand sequence being SEQ ID NO: 117; siRNA-57, with the sense strand sequence being SEQ ID NO: 57 and the antisense strand sequence being SEQ ID NO: 118; siRNA-58, with the sense strand sequence being SEQ ID NO: 58 and the antisense strand sequence being SEQ ID NO: 119; siRNA-59, with the sense strand sequence being SEQ ID NO: 59 and the antisense strand sequence being SEQ ID NO: 120; siRNA-60, with the sense strand sequence being SEQ ID NO: 60 and the antisense strand sequence being SEQ ID NO: 121; siRNA-61, with the sense strand sequence being SEQ ID NO: 61 and the antisense strand sequence being SEQ ID NO: 122.

In some embodiments, the antisense strand contains at least 15, 16, 17, 18, 19, 20, 21, 22, or 23 consecutive nucleotides differing by no more than 0, 1, 2, or 3 nucleotides from any at least 15, 16, 17, 18, 19, 20, 21, 22, or 23 consecutive nucleotides in any of the sequences shown in SEQ ID NO: 62-122; the sense strand contains a nucleotide sequence that is at least partially complementary (e.g., partially complementary, substantially complementary, or fully complementary) to the antisense strand.

In some embodiments, the sense strand contains at least 15, 16, 17, 18, 19, 20, or 21 consecutive nucleotides differing by no more than 0, 1, 2, or 3 nucleotides from any at least 15, 16, 17, 18, 19, 20, or 21 consecutive nucleotides in any of the sequences shown in SEQ ID NO: 1-61.

In some embodiments, the antisense strand contains a nucleotide sequence differing by 0, 1, or 2 nucleotides from any of the sequences shown in SEQ ID NO: 79, 81, 83, 85, 96, 103, 113, and the sense strand contains a nucleotide sequence that is at least partially complementary (e.g., partially complementary, substantially complementary, or fully complementary) to the antisense strand.

In some embodiments, the sense strand contains a nucleotide sequence differing by 0, 1, or 2 nucleotides from any of the sequences shown in SEQ ID NO: 18, 20, 22, 24, 35, 42, 52.

In some preferred embodiments, the sense strand contains a nucleotide sequence differing by 0, 1, or 2 nucleotides from the sequence shown in SEQ ID NO: 18, and the antisense strand contains a nucleotide sequence differing by 0, 1, or 2 nucleotides from the sequence shown in SEQ ID NO: 79.

In some more preferred embodiments, the sense strand contains a nucleotide sequence differing by 0, 1, or 2 nucleotides from the sequence shown in SEQ ID NO: 20, and the antisense strand contains a nucleotide sequence differing by 0, 1, or 2 nucleotides from the sequence shown in SEQ ID NO: 81.

In some preferred embodiments, the sense strand contains a nucleotide sequence differing by 0, 1, or 2 nucleotides from the sequence shown in SEQ ID NO: 22, and the antisense strand contains a nucleotide sequence differing by 0, 1, or 2 nucleotides from the sequence shown in SEQ ID NO: 83.

In some preferred embodiments, the sense strand contains a nucleotide sequence differing by 0, 1, or 2 nucleotides from the sequence shown in SEQ ID NO: 24, and the antisense strand contains a nucleotide sequence differing by 0, 1, or 2 nucleotides from the sequence shown in SEQ ID NO: 85.

In some preferred embodiments, the sense strand contains a nucleotide sequence differing by 0, 1, or 2 nucleotides from the sequence shown in SEQ ID NO: 35, and the antisense strand contains a nucleotide sequence differing by 0, 1, or 2 nucleotides from the sequence shown in SEQ ID NO: 96.

In some preferred embodiments, the sense strand contains a nucleotide sequence differing by 0, 1, or 2 nucleotides from the sequence shown in SEQ ID NO: 42, and the antisense strand contains a nucleotide sequence differing by 0, 1, or 2 nucleotides from the sequence shown in SEQ ID NO: 103.

In some preferred embodiments, the sense strand contains a nucleotide sequence differing by 0, 1, or 2 nucleotides from the sequence shown in SEQ ID NO: 52, and the antisense strand contains a nucleotide sequence differing by 0, 1, or 2 nucleotides from the sequence shown in SEQ ID NO: 113.

All nucleotide groups in the above nucleic acid may be unmodified or contain at least one modified nucleotide group, and the modification may be on a nucleotide at any position.

In some embodiments, when the sense strand or antisense strand of the nucleic acid has a sequence identity of less than 100% to the corresponding sequence mentioned in the present disclosure or differs by more than one nucleotide, it still exhibits a similar (e.g., still having 80-120%, 85-115%, or 90-110% of the efficacy of the corresponding sequence) or comparable (e.g., still having 95-105% of the efficacy of the corresponding sequence) inhibitory effect on MAPT (Tau). For example, replacing the two bases at the 3′ end of the antisense strand (such as any sequence shown in SEQ ID NO: 62-122) with AA, CU, UC, AG, CC, GG, UG, etc., or any combination of two nucleotides. Such nucleic acid sequences also fall within the protection scope of the present disclosure.

In some preferred embodiments, the inhibitory efficiency of the nucleic acid on MAPT (Tau) is not less than 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%).

TABLE 1

| Target | | SEQ ID | | SEQ ID |
|---|---|---|---|---|
| position | Sense (5′-3′) | NO: | Antisense (5′-3′) | NO: |
| 166 | CAGGAGUUCGAAGUGAU | 1 | UUCCAUCACUUCGAACUCC | 62 |
| | GGAA | | UGGC | |
| 235 | UACACCAUGCACCAAGA | 2 | UUGGUCUUGGUGCAUGGUG | 63 |
| | CCAA | | UAGC | |
| 266 | CGGACGCUGGCCUGAAA | 3 | AUUCUUUCAGGCCAGCGUC | 64 |
| | GAAU | | CGUG | |
| 343 | GAUGCUAAGAGCACUCC | 4 | UGUUGGAGUGCUCUUAGCA | 65 |
| | AACA | | UCAG | |
| 1181 | UGGAUUUCCUCUCCAAA | 5 | UAACUUUGGAGAGGAAAUC | 66 |
| | GUUA | | CACA | |
| 1277 | UCACGUUUCACGUGGAA | 6 | UGAUUUCCACGUGAAACGU | 67 |
| | AUCA | | GAAC | |
| 1529 | GGACUGGAAGCGAUGAC | 7 | UUUUGUCAUCGCUUCCAGU | 68 |
| | AAAA | | CCUU | |
| 2144 | GGAAGGUGCAGAUAAUU | 8 | UAUUAAUUAUCUGCACCUU | 69 |
| | AAUA | | CCCG | |
| 2147 | AGGUGCAGAUAAUUAAU | 9 | UCUUAUUAAUUAUCUGCAC | 70 |
| | AAGA | | CUUC | |
| 2151 | GCAGAUAAUUAAUAAGA | 10 | UGCUUCUUAUUAAUUAUCU | 71 |
| | AGCA | | GCAC | |
| 2175 | UCUUAGCAACGUCCAGU | 11 | UUGGACUGGACGUUGCUAA | 72 |
| | CCAA | | GAUC | |
| 2194 | AAGUGUGGCUCAAAGGA | 12 | AUUAUCCUUUGAGCCACAC | 73 |
| | UAAU | | UUGG | |
| 2199 | GGCUCAAAGGAUAAUAU | 13 | UUUGAUAUUAUCCUUUGAG | 74 |
| | CAAA | | CCUU | |
| 2202 | CUCAAAGGAUAAUAUCA | 14 | UGUUUGAUAUUAUCCUUUG | 75 |
| | AACA | | AGCC | |
| 2238 | CAGUGUGCAAAUAGUCU | 15 | UUGUAGACUAUUUGCACAC | 76 |
| | ACAA | | UGCC | |
| 2327 | GUGGCCAGGUGGAAGUA | 16 | AUUUUACUUCCACCUGGCC | 77 |
| | AAAU | | ACCU | |
| 2328 | UGGCCAGGUGGAAGUAA | 17 | UAUUUUACUUCCACCUGGC | 78 |
| | AAUA | | CACC | |
| 2421 | CGGAGGAAAUAAAAAGA | 18 | UCAAUCUUUUUAUUUCCUC | 79 |
| | UUGA | | CGCC | |
| 2422 | GGAGGAAAUAAAAAGAU | 19 | UCAAUCUUUUUAUUUCCUC | 80 |
| | UGAA | | CUUU | |
| 2423 | GAGGAAAUAAAAAGAUU | 20 | UUUCAAUCUUUUUAUUUCC | 81 |
| | GAAA | | UCCG | |
| 2633 | UGGCCAAGCAGGGUUUG | 21 | AUCACAAACCCUGCUUGGC | 82 |
| | UGAU | | CAGG | |
| 2811 | GCUCGGGACUUCAAAAU | 22 | ACUGAUUUUGAAGUCCCGA | 83 |
| | CAGU | | GCUU | |
| 2938 | GGCAAUUCCUUUUGAUU | 23 | AAAGAAUCAAAAGGAAUUG | 84 |
| | CUUU | | CCUU | |
| 3085 | AGCAACAAAGGAUUUGA | 24 | AGUUUCAAAUCCUUUGUUG | 85 |
| | AACU | | CUUU | |
| 3086 | GCAACAAAGGAUUUGAA | 25 | AAGUUUCAAAUCCUUUGUU | 86 |
| | ACUU | | GCUU | |
| 3565 | UCCACAGAAACCCUGUU | 26 | AUAAAACAGGGUUUCUGUG | 87 |
| | UUAU | | GAUU | |
| 3566 | CCACAGAAACCCUGUUU | 27 | AAUAAAACAGGGUUUCUGU | 88 |
| | UAUU | | GGAU | |
| 3936 | CUCCAUACUGAGGGUGA | 28 | AAUUUCACCCUCAGUAUGG | 89 |
| | AAUU | | AGUU | |
| 3937 | UCCAUACUGAGGGUGAA | 29 | UAAUUUCACCCUCAGUAUG | 90 |
| | AUUA | | GAUU | |
| 3938 | CCAUACUGAGGGUGAAA | 30 | UUAAUUUCACCCUCAGUAU | 91 |
| | UUAA | | GGAG | |
| 4454 | GCAGCUGAACAUAUACA | 31 | UCUAUGUAUAUGUUCAGCU | 92 |
| | UAGA | | GCUU | |
| 4458 | CUGAACAUAUACAUAGA | 32 | AACAUCUAUGUAUAUGUUC | 93 |
| | UGUU | | AGUU | |
| 4507 | GAGUUGUAGUUGGAUUU | 33 | AGACAAAUCCAACUACAAC | 94 |
| | GUCU | | UCAA | |
| 4509 | GUUGUAGUUGGAUUUGU | 34 | ACAGACAAAUCCAACUACA | 95 |
| | CUGU | | ACUC | |
| 4516 | UUGGAUUUGUCUGUUUA | 35 | AGCAUAAACAGACAAAUCC | 96 |
| | UGCU | | AAUU | |
| 4517 | UGGAUUUGUCUGUUUAU | 36 | AAGCAUAAACAGACAAAUC | 97 |
| | GCUU | | CAAA | |
| 4518 | GGAUUUGUCUGUUUAUG | 37 | AAAGCAUAAACAGACAAAU | 98 |
| | CUUU | | CCAA | |
| 4548 | GAGUGACUAUGAUAGUG | 38 | UUUUCACUAUCAUAGUCAC | 99 |
| | AAAA | | UCUU | |
| 4550 | GUGACUAUGAUAGUGAA | 39 | UCUUUUCACUAUCAUAGUC | 100 |
| | AAGA | | ACUC | |
| 4558 | GAUAGUGAAAAGAAAAA | 40 | UUUUUUUUUCUUUUCACUA | 101 |
| | AAAA | | UCAU | |
| 4591 | CGCAUGUAUCUUGAAAU | 41 | AAGCAUUUCAAGAUACAUG | 102 |
| | GCUU | | CGUU | |
| 4593 | CAUGUAUCUUGAAAUGC | 42 | ACAAGCAUUUCAAGAUACA | 103 |
| | UUGU | | UGCG | |
| 4598 | AUCUUGAAAUGCUUGUA | 43 | UCUUUACAAGCAUUUCAAG | 104 |
| | AAGA | | AUUU | |
| 4795 | GAAGCACAGGAUUAGGA | 44 | UCAGUCCUAAUCCUGUGCU | 105 |
| | CUGA | | UCUU | |
| 4957 | GCUUACAACUCCUGCAU | 45 | UGUGAUGCAGGAGUUGUAA | 106 |
| | CACA | | GCUU | |
| 5385 | GAGGGACUUGGCAGUAG | 46 | AUUUCUACUGCCAAGUCCC | 107 |
| | AAAU | | UCUU | |
| 5896 | GUGGGCUAGAUAGGAUA | 47 | AGUAUAUCCUAUCUAGCCC | 108 |
| | UACU | | ACUU | |
| 5898 | GGGCUAGAUAGGAUAUA | 48 | ACAGUAUAUCCUAUCUAGC | 109 |
| | CUGU | | CCUU | |
| 5899 | GGCUAGAUAGGAUAUAC | 49 | UACAGUAUAUCCUAUCUAG | 110 |
| | UGUA | | CCUU | |
| 5900 | GCUAGAUAGGAUAUACU | 50 | AUACAGUAUAUCCUAUCUA | 111 |
| | GUAU | | GCUU | |
| 5951 | AUCAAUAGUUCCAUUUA | 51 | AAUUUAAAUGGAACUAUUG | 112 |
| | AAUU | | AUUU | |
| 5953 | CAAUAGUUCCAUUUAAA | 52 | UCAAUUUAAAUGGAACUAU | 113 |
| | UUGA | | UGAU | |
| 6598 | CCCAUGAGUUUGCCAUG | 53 | UCAACAUGGCAAACUCAUG | 114 |
| | UUGA | | GGUU | |
| 6646 | CCCAUGAUUUCUUCGGU | 54 | AAUUACCGAAGAAAUCAUG | 115 |
| | AAUU | | GGUU | |
| 6713 | GUCUGUGAAUGUCUAUA | 55 | ACUAUAUAGACAUUCACAG | 116 |
| | UAGU | | ACUU | |
| 6715 | GUGAAUGUCUAUAUAGU | 56 | ACACUAUAUAGACAUUCAC | 117 |
| | GUAU | | AGAC | |
| 6717 | GUGAAUGUCUAUAUAGU | 57 | AUACACUAUAUAGACAUUC | 118 |
| | GUAU | | ACUU | |
| 6732 | GUGUAUUGUGUGUUUUA | 58 | UUGUUAAAACACACAAUAC | 119 |
| | ACAA | | ACUU | |
| 6734 | GUAUUGUGUGUUUUAAC | 59 | AUUUGUUAAAACACACAAU | 120 |
| | AAAU | | ACAC | |
| 6737 | UUGUGUGUUUUAACAAA | 60 | AUCAUUUGUUAAAACACAC | 121 |
| | UGAU | | AAUU | |
| 6739 | GUGUGUUUUAACAAAUG | 61 | AAAUCAUUUGUUAAAACAC | 122 |
| | AUUU | | ACAA | |

In Table 1, the first column refers to the position of the first base of the target gene in the coding sequence of MAPT, and so on; the numbers in the 3rd and 5th columns represent sequence numbers, for example, “1” represents SEQ ID NO: 1. The reference sequence of the target gene is the coding sequence NM_001377265.1 of human MAPT, as shown in SEQ ID NO: 123.

In some embodiments, the sense strand in the nucleic acid contains a sequence with at least 80% (e.g., 80%, 81%, 82%, 83%, 84%, 85%, 86%, 87%, 88%, 89%, 90%, 91%, 92%, 93%, 94%, 95%, 96%, 97%, 98%, 99%) sequence identity to the sequence of equal length in nucleotides 100-400, 1100-1600, 2100-3200, 3500-3600, 3900-4000, 4400-5000, 5300-5400, 5800-6000, or 6500-6800 of SEQ ID NO:123.

In some embodiments, the nucleic acid targets any position within the regions of nucleotides 100-400, 1100-1600, 2100-3200, 3500-3600, 3900-4000, 4400-5000, 5300-5400, 5800-6000, or 6500-6800 of SEQ ID NO:123.

In some embodiments, the nucleic acid targets any position within the regions of nucleotides 2400-3150, 4500-4650, or 5900-6000 of SEQ ID NO:123.

In some embodiments, the nucleic acid targets any position within the regions of nucleotides 2400-2500, 2750-2850, 3050-3150, 4450-4550, 4550-4650, or 5900-6000 of SEQ ID NO:123.

In some embodiments, the nucleic acid targets any position within the region of nucleotides 2410-2460 of SEQ ID NO:123.

In some embodiments, the nucleic acid targets any position within the region of nucleotides 2800-2850 of SEQ ID NO:123.

In some embodiments, the nucleic acid targets any position within the region of nucleotides 3070-3120 of SEQ ID NO:123.

In some embodiments, the nucleic acid targets any position within the region of nucleotides 4500-4550 of SEQ ID NO:123.

In some embodiments, the nucleic acid targets any position within the region of nucleotides 4580-4630 of SEQ ID NO:123.

In some embodiments, the nucleic acid targets any position within the region of nucleotides 5940-5990 of SEQ ID NO:123.

The advantageous effects of the above-mentioned technical solutions regarding the naked sequence (i.e., unmodified sequence) in the present disclosure do not depend on the selection of modification methods or targeting vectors. The applicable modification schemes and further preferred modification schemes are elaborated below.

In some embodiments, the nucleic acid contains nucleotide groups as basic structural units, and the nucleotide groups contain a phosphate group, a ribose group, and a base. Preferably, the nucleic acid contains at least one modified nucleotide group. The nucleic acid containing modified group(s) has an inhibitory efficiency on MAPT of not less than 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%).

In some embodiments, the modified nucleotide group is a nucleotide group in which the phosphate group and/or ribose group is modified. The modified sites may be on at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the nucleotides at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 of the sense strand and/or antisense strand.

In some embodiments, the modification of the phosphate group refers to the modification of oxygen in the phosphate group, including phosphorthioate modification, boranophosphate modification, etc. As shown in the following formula, oxygen in the phosphate group is replaced by sulfur, borane, amino, alkyl, or alkoxy. These modifications can stabilize the structure of nucleic acids and maintain high specificity and high affinity of base pairing.

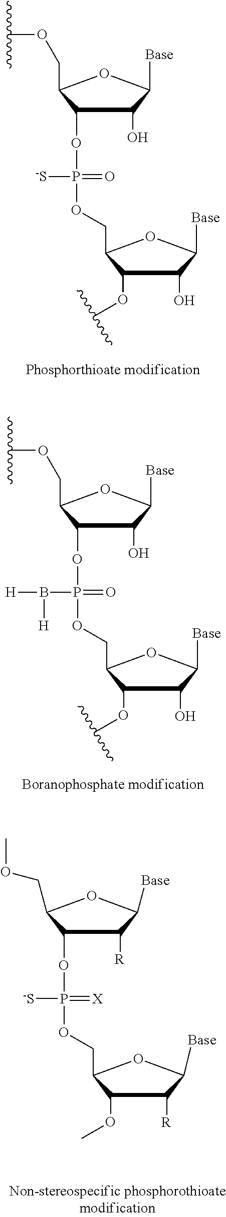

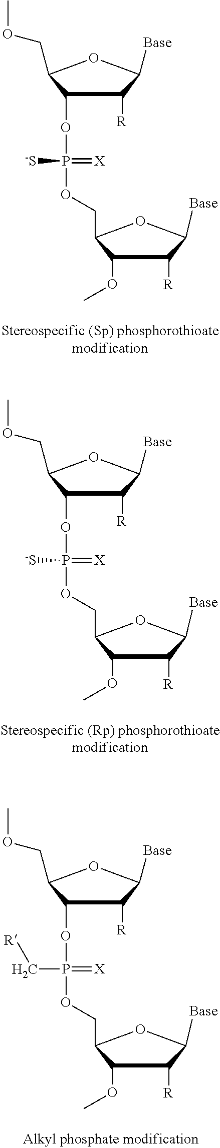

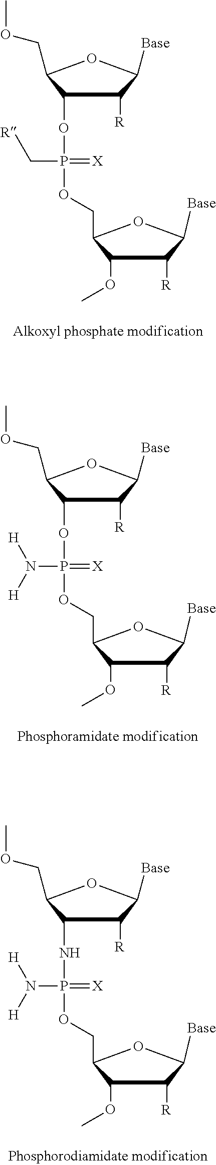

In the above structural formula, Base denotes the base A, U, C, G or T. X may be oxygen (O) or sulfur (S). R may be the same or different in the above structure, such as hydrogen (H), fluorine (F), methoxyl (OME) or methoxyethyl (MOE), hydroxyl, allyl, ethylamino, propargyl, amino, cyanoethyl, acetyl etc.; R′ and R″ may each independently be hydrogen (H), methyl (CH3), ethyl (CH₂CH₃), propyl (CH₂CH₂CH₃), isopropyl (CH(CH₃)₂), allyl, propargyl, acyloxybenzyl and acyloxyethyl.

Modification of the ribose group refers to modification of the 2′-hydroxyl group (2′-OH) in the ribose group. After introducing certain substituents (e.g., methoxyl or fluorine) at the 2′-hydroxyl of the ribose group, the nucleic acid is not readily cleaved by ribonuclease, thereby enhancing stability of the nucleic acid and allowing the nucleic acid to be more resistant to hydrolysis by the nuclease. Modifications of the 2′-hydroxyl group in the nucleotide pentose include 2′-fluoro modification (e.g., 2′-arabino-fluoro modification), 2′-methoxy modification (2′-OME), 2′-methoxyethyl modification (2′-MOE), 2′-2,4-dinitrophenol modification (2′-DNP modification), 2′,4′-constrained ethyl modification, 2′-Amino modification, 2′-Deoxy modification, BNA, acyclic nucleic acid modification, mal-positioned nucleic acid modification, L-type nucleic acid modification and the like. BNA (internal ring bridged nucleotide) refers to a constrained or inaccessible nucleotide. BNA can contain a bridging structure of a five-, six-, or seven-membered ring, with a “locked” C3′-endo sugar pucker. The bridge is typically incorporated into the 2′-, 4′-position of the ribose ring to provide the 2′,4′-BNA nucleotides such as locked ethyl modification (LNA), constrained ethyl modification (ENA), and constrained ethyl bicyclic nucleic acid modification (cET BNA). Acyclic nucleic acid is a nucleotide formed after the ribose ring of the nucleotide is opened, such as unlocked nucleic acid (UNA) nucleotides and glycerol nucleic acid (GNA) nucleotides. Mal-positioned nucleic acid modification refers to a 3′,5′-phosphate bond linkage substituted by the 2′,5′-phosphate bond linkage. L-type nucleic acid modification refers to a naturally occurring D-type nucleic acid being substituted by its mirror-stereoscopic counterpart L-type nucleic acid.

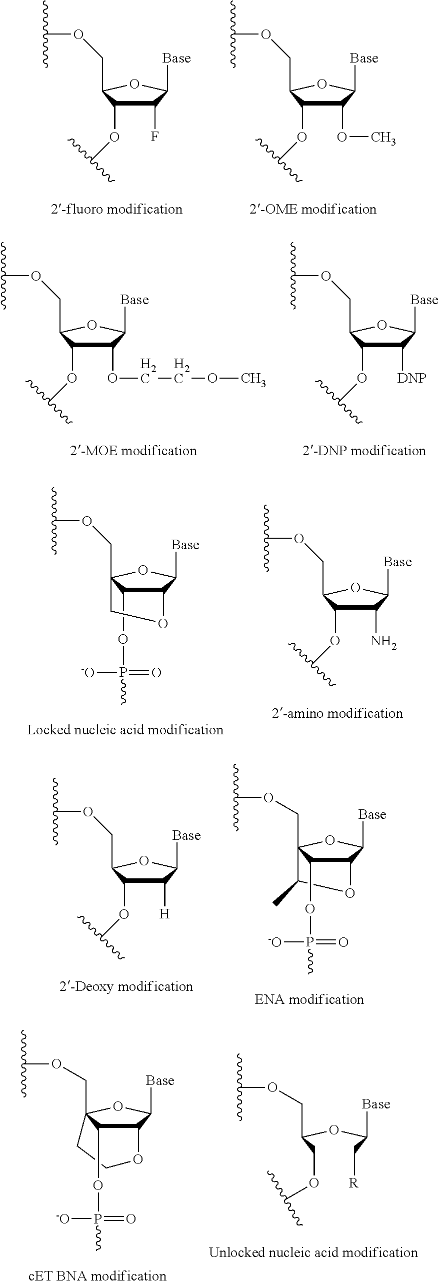

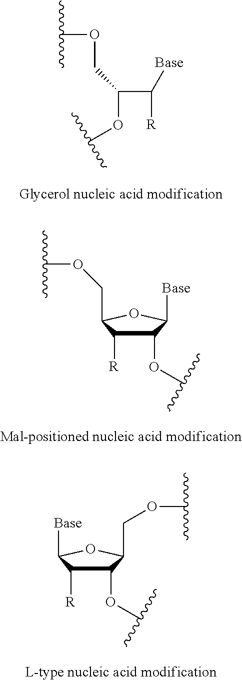

In the above structural formula, Base denotes the base A, U, C, G or T. R may be the same or different in the above structure, such as hydrogen (H), fluorine (F), methoxyl (OME) or methoxyethyl (MOE), hydroxyl, allyl, ethylamino, propargyl, cyanoethyl and acetyl.

According to a particularly preferred embodiment of the present disclosure, wherein the sense strand of the RNAi agent comprising the nucleotide group of uracil base or cytosine base, which is the nucleotide group in which the ribose group is modified, that is, the 2′-OH of the ribose group in the sense strand of the RNAi agent comprising the nucleotide group of uracil base or cytosine base is substituted by methoxyl or fluorine. More preferably, the 3′-end of both the sense strand and antisense strand of the RNAi agent may be linked with dTdT; alternatively, the 3′-end of the antisense strand of the RNAi agent may be linked with AA or UU, or any combination of two nucleic acids (the nucleic acids may be but are not limited to CC, GG or UG), to provide the sequence with specificity as inducement to mRNA degradation. RNAi agents with such modifications exhibit more excellent in vivo inhibitory effect, and said modifications may further reduce the in vivo immunogenicity of the RNAi agents of the present disclosure.

The RNAi agent of the present disclosure may further comprise the modification in which a monophosphate nucleoside is linked to the 5′-end of the antisense strand. The 5′-monophosphate at the end of the guide strand of the siRNA is important for RISC recognition. Wherein phosphorylation of the 5′-hydroxyl group plays a certain role on whether the siRNA can be effectively loaded on the intracellular Ago2. The monophosphate at 5′-end of the guide strand in the siRNA has interaction with Argonaute-2 (Ago2) through the hydrogen bond, in order to ensure accurate targeting and precise cleavage of the mRNA target. Several derivatives of the 5′-monophosphate nucleosides are commonly used, this type of derivative of the phosphate nucleoside has been proven to exhibit certain stability in the biological metabolism medium, and to play a certain role in facilitating the loading of siRNA guide strand on the intracellular Ago2 (Nucleic Acids Research, 2015, 43, 2993-3011). The RNAi agent according to the present disclosure, wherein the trans-vinyl phosphate (VP) is preferably the first choice, the RNAi agent may comprise derivatives of the monophosphate nucleoside other than those mentioned above.

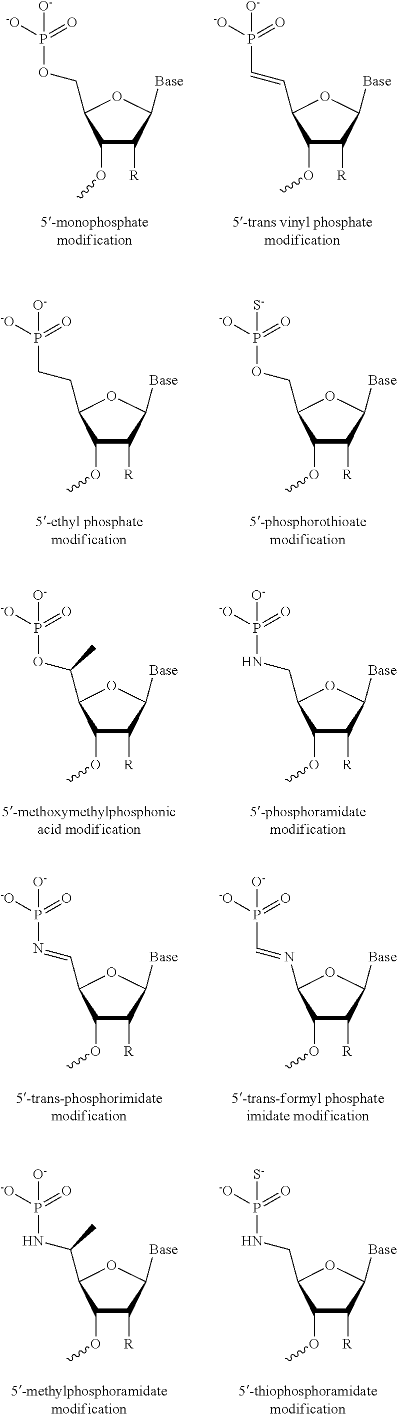

In the above structural formula, Base denotes the base A, U, C, G or T. R may be the same or different in the above structure, such as hydrogen (H), fluorine (F), methoxyl (OME) or 5 methoxyethyl (MOE), hydroxyl, allyl, ethylamino, propargyl, cyanoethyl, amino and acetyl.

In the present disclosure, the meanings of

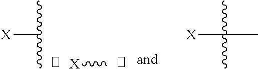

are consistent, referring to a chemical element X that is connected with any one or more groups.

In some embodiments, at least one nucleotide in the nucleic acid is a modified nucleotide or includes a modified internucleotide linkage.

In some embodiments, the modified nucleotide is selected from one or more of 2′-O-methylnucleotides, 2′-fluoronucleotides, 2′-deoxyribonucleotides, 2′,3′-open cyclic nucleotide analogs, locked nucleotides, 2′-F-arabinose nucleotide, 2′-methoxyethyl nucleotide, dealkylated nucleotide, ribitol, reverse nucleotide, reverse 2′-O-methyl nucleotide, reverse 2′-deoxyribonucleotides, 2′-amino-modified nucleotides, 2′-alkyl-modified nucleotides, morpholino, peptide nucleic acid (PNA), glycerol nucleic acid (GNA), triazacyclic DNA (tcDNA), nucleotides comprising unnatural bases, nucleotides containing vinyl phosphonates, nucleotides containing cyclopropyl phosphonates, and 3′-O-methyl nucleotides. In some embodiments, the modified nucleotide is further preferably selected from one or two of 2′-O-methylnucleotides and 2′-fluoronucleotides.

In some embodiments, the modified internucleotide linkage is preferably selected from one or more of phosphorothioate internucleotide linkages and methylphosphonate internucleotide linkages. In some embodiments, the modified internucleotide linkage is further preferably selected from one or more of phosphorothioate monoester internucleotide linkages and phosphorothioate diester internucleotide linkages.

In some preferred embodiments, the antisense strand has a 2′-fluoronucleotide at positions 2, 4, 6, and 16, and optionally at position 14, of any nucleotide sequence shown in SEQ ID NO: 62-122. Compared with known modification methods, the above-mentioned antisense strand modification scheme is further conducive to enhancing the inhibitory effect of the nucleic acid on MAPT.

In some more preferred embodiments, the antisense strand has a 2′-fluoronucleotide at positions 2, 4, 6, and 16, and optionally at position 14, of any nucleotide sequence shown in SEQ ID NO: 62-122, with the remaining nucleotides being 2′-O-methylnucleotides.

In some preferred embodiments, the sense strand has a 2′-fluoronucleotide at positions 7, 9, 10, and 11, and optionally at position 18, of any nucleotide sequence shown in SEQ ID NO: 1-61. Compared with known modification methods, the above-mentioned sense strand modification scheme is further conducive to enhancing the inhibitory effect of the nucleic acid on MAPT.

In some more preferred embodiments, the sense strand has a 2′-fluoronucleotide at positions 7, 9, 10, and 11, and optionally at position 18, of any nucleotide sequence shown in SEQ ID NO: 1-61, with the remaining nucleotides being 2′-O-methylnucleotides.

In some embodiments, the antisense strand comprises a phosphorothioate internucleotide linkage between the last 2-4 (e.g., 2, 3, or 4) nucleotides at the 5′ end and/or the 3′ end of the antisense strand, and the sense strand comprises a phosphorothioate internucleotide linkage between the last 2-4 (e.g., 2, 3, or 4) nucleotides at the 5′ end and/or the 3′ end of the sense strand.

In some specific embodiments, the antisense strand has phosphorothioate internucleotide linkages between the last 3 nucleotides at the 5′ end and 3′ end of the antisense strand. The sense strand has phosphorothioate internucleotide linkages between the last 3 nucleotides at the 5′ end of the sense strand.

In some embodiments, the antisense strand contains a nucleotide sequence differing by 0, 1, or 2 nucleotides from any antisense strand shown in Table 2 or Table 3.

In some embodiments, the sense strand contains a nucleotide sequence differing by 0, 1, or 2 nucleotides from any sense strand shown in Table 2 or Table 3.

In some embodiments, the nucleic acid contains a double strand shown in either Table 2 or Table 3.

In some preferred embodiments, the nucleic acid contains a double strand selected from any one of SN-5180, SN-5182, SN-5194, SN-5196, SN-5107, SN-5114, SN-5124, SN-6725, and SN-6726.

TABLE 2

| No. | Sense (5′-3′) | Antisense (5′-3′) |
|---|---|---|
| SN-5161 | csasggagUfuCfGfAfagugauggaa | usUfscCfaUfcacuucgAfaCfuccugsgsc |
| SN-5162 | usascaccAfuGfCfAfccaagaccaa | usUfsgGfuCfuuggugcAfuGfguguasgsc |
| SN-5173 | csgsgacgCfuGfGfCfcugaaagaau | asUfsuCfuUfucaggccAfgCfguccgsusg |
| SN-5174 | gsasugcuAfaGfAfGfcacuccaaca | usGfsuUfgGfagugcucUfuAfgcaucsasg |
| SN-5175 | usgsgauuUfcCfUfCfuccaaaguua | usAfsaCfuUfuggagagGfaAfauccascsa |
| SN-5176 | uscsacguUfuCfAfCfguggaaauca | usGfsaUfuUfccacgugAfaAfcgugasasc |
| SN-5177 | gsgsacugGfaAfGfCfgaugacaaaa | usUfsuUfgUfcaucgcuUfcCfaguccsusu |
| SN-5178 | gsgsaaggUfgCfAfGfauaauuaaua | usAfsuUfaAfuuaucugCfaCfcuuccscsg |
| SN-5179 | asgsgugcAfgAfUfAfauuaauaaga | usCfsuUfaUfuaauuauCfuGfcaccususc |
| SN-5170 | gscsagauAfaUfUfAfauaagaagca | usGfscUfuCfuuauuaaUfuAfucugcsasc |
| SN-5171 | uscsuuagCfaAfCfGfuccaguccaa | usUfsgGfaCfuggacguUfgCfuaagasusc |
| SN-5172 | asasguguGfgCfUfCfaaaggauaau | asUfsuAfuCfcuuugagCfcAfcacuusgsg |
| SN-5183 | gsgscucaAfaGfGfAfuaauaucaaa | usUfsuGfaUfauuauccUfuUfgagccsusu |
| SN-5185 | csuscaaaGfgAfUfAfauaucaaaca | usGfsuUfuGfauauuauCfcUfuugagscsc |
| SN-5186 | csasguguGfcAfAfAfuagucuacaa | usUfsgUfaGfacuauuuGfcAfcacugscsc |
| SN-5187 | gsusggccAfgGfUfGfgaaguaaaau | asUfsuUfuAfcuuccacCfuGfgccacscsu |
| SN-5188 | usgsgccaGfgUfGfGfaaguaaaaua | usAfsuUfuUfacuuccaCfcUfggccascsc |
| SN-5180 | csgsgaggAfaAfUfAfaaaagauuga | usCfsaAfuCfuuuuuauUfuCfcuccgscsc |
| SN-5181 | gsgsaggaAfaUfAfAfaaagauugaa | usCfsaAfuCfuuuuuauUfuCfcuccususu |
| SN-5182 | gsasggaaAfuAfAfAfaagauugaaa | usUfsuCfaAfucuuuuuAfuUfuccucscsg |
| SN-5193 | usgsgccaAfgCfAfGfgguuugugau | asUfscAfcAfaacccugCfuUfggccasgsg |
| SN-5194 | gscsucggGfaCfUfUfcaaaaucagu | asCfsuGfaUfuuugaagUfcCfcgagcsusu |
| SN-5195 | gsgscaauUfcCfUfUfuugauucuuu | asAfsaGfaAfucaaaagGfaAfuugccsusu |
| SN-5196 | asgscaacAfaAfGfGfauuugaaacu | asGfsuUfuCfaaauccuUfuGfuugcususu |
| SN-5197 | gscsaacaAfaGfGfAfuuugaaacuu | asAfsgUfuUfcaaauccUfuUfguugcsusu |
| SN-5198 | uscscacaGfaAfAfCfccuguuuuau | asUfsaAfaAfcaggguuUfcUfguggasusu |
| SN-5199 | cscsacagAfaAfCfCfcuguuuuauu | asAfsuAfaAfacaggguUfuCfuguggsasu |
| SN-5190 | csusccauAfcUfGfAfgggugaaauu | asAfsuUfuCfacccucaGfuAfuggagsusu |
| SN-5191 | uscscauaCfuGfAfGfggugaaauua | usAfsaUfuUfcacccucAfgUfauggasusu |
| SN-5192 | cscsauacUfgAfGfGfgugaaauuaa | usUfsaAfuUfucacccuCfaGfuauggsasg |
| SN-5103 | gscsagcuGfaAfCfAfuauacauaga | usCfsuAfuGfuauauguUfcAfgcugcsusu |
| SN-5104 | csusgaacAfuAfUfAfcauagauguu | asAfscAfuCfuauguauAfuGfuucagsusu |
| SN-5105 | gsasguugUfaGfUfUfggauuugucu | asGfsaCfaAfauccaacUfaCfaacucsasa |
| SN-5106 | gsusuguaGfuUfGfGfauuugucugu | asCfsaGfaCfaaauccaAfcUfacaacsusc |
| SN-5107 | ususggauUfuGfUfCfuguuuaugcu | asGfscAfuAfaacagacAfaAfuccaasusu |
| SN-5108 | usgsgauuUfgUfCfUfguuuaugcuu | asAfsgCfaUfaaacagaCfaAfauccasasa |
| SN-5109 | gsgsauuuGfuCfUfGfuuuaugcuuu | asAfsaGfcAfuaaacagAfcAfaauccsasa |
| SN-5100 | gsasgugaCfuAfUfGfauagugaaaa | usUfsuUfcAfcuaucauAfgUfcacucsusu |
| SN-5101 | gsusgacuAfuGfAfUfagugaaaaga | usCfsuUfuUfcacuaucAfuAfgucacsusc |
| SN-5102 | gsasuaguGfaAfAfAfgaaaaaaaaa | usUfsuUfuUfuuucuuuUfcAfcuaucsasu |
| SN-5113 | csgscaugUfaUfCfUfugaaaugcuu | asAfsgCfaUfuucaagaUfaCfaugcgsusu |
| SN-5114 | csasuguaUfcUfUfGfaaaugcuugu | asCfsaAfgCfauuucaaGfaUfacaugscsg |
| SN-5115 | asuscuugAfaAfUfGfcuuguaaaga | usCfsuUfuAfcaagcauUfuCfaagaususu |
| SN-5116 | gsasagcaCfaGfGfAfuuaggacuga | usCfsaGfuCfcuaauccUfgUfgcuucsusu |
| SN-5117 | gscsuuacAfaCfUfCfcugcaucaca | usGfsuGfaUfgcaggagUfuGfuaagcsusu |
| SN-5118 | gsasgggaCfuUfGfGfcaguagaaau | asUfsuUfcUfacugccaAfgUfcccucsusu |
| SN-5119 | gsusgggcUfaGfAfUfaggauauacu | asGfsuAfuAfuccuaucUfaGfcccacsusu |
| SN-5110 | gsgsgcuaGfaUfAfGfgauauacugu | asCfsaGfuAfuauccuaUfcUfagcccsusu |
| SN-5111 | gsgscuagAfuAfGfGfauauacugua | usAfscAfgUfauauccuAfuCfuagccsusu |
| SN-5112 | gscsuagaUfaGfGfAfuauacuguau | asUfsaCfaGfuauauccUfaUfcuagcsusu |
| SN-5123 | asuscaauAfgUfUfCfcauuuaaauu | asAfsuUfuAfaauggaaCfuAfuugaususu |
| SN-5124 | csasauagUfuCfCfAfuuuaaauuga | usCfsaAfuUfuaaauggAfaCfuauugsasu |
| SN-5125 | cscscaugAfgUfUfUfgccauguuga | usCfsaAfcAfuggcaaaCfuCfaugggsusu |
| SN-5126 | cscscaugAfuUfUfCfuucgguaauu | asAfsuUfaCfcgaagaaAfuCfaugggsusu |
| SN-5127 | gsuscuguGfaAfUfGfucuauauagu | asCfsuAfuAfuagacauUfcAfcagacsusu |
| SN-5128 | gsusgaauGfuCfUfAfuauaguguau | asCfsaCfuAfuauagacAfuUfcacagsasc |
| SN-5129 | gsusgaauGfuCfUfAfuauaguguau | asUfsaCfaCfuauauagAfcAfuucacsusu |
| SN-5120 | gsusguauUfgUfGfUfguuuuaacaa | usUfsgUfuAfaaacacaCfaAfuacacsusu |
| SN-5121 | gsusauugUfgUfGfUfuuuaacaaau | asUfsuUfgUfuaaaacaCfaCfaauacsasc |
| SN-5122 | ususguguGfuUfUfUfaacaaaugau | asUfscAfuUfuguuaaaAfcAfcacaasusu |
| SN-5233 | gsusguguUfuUfAfAfcaaaugauuu | asAfsaUfcAfuuuguuaAfaAfcacacsasa |

TABLE 3

| No. | Sense (5′-3′) | Antisense (5′-3′) |
|---|---|---|
| SN-6725 | gsasggaaAfuAfAfAfaagauuGfaaa | usUfsuCfaAfucuuuuuAfuUfuccucscsg |
| SN-6726 | gsasggaaAfuAfAfAfaagauugaaa | usUfsuCfaAfucuuuuuauUfuccucscsg |
| SN-5182 | gsasggaaAfuAfAfAfaagauugaaa | usUfsuCfaAfucuuuuuAfuUfuccucscsg |

In each modified sequence of the present disclosure, nucleotides represented by lowercase letters are 2′-O-methylnucleotides; “f” indicates that the adjacent nucleotide to its left is a 2′-fluoronucleotide; and “s” represents that the two adjacent nucleotides on its left and right are linked via a phosphorothioate diester internucleotide linkage.

The nucleic acids described in the present disclosure can be obtained by conventional methods in the art, such as solid-phase synthesis and liquid-phase synthesis. Commercial custom services for solid-phase synthesis are available, so they can also be obtained through commercial purchase. The modified nucleotide groups can be introduced via nucleotide monomers with corresponding modifications.

Based on the nucleic acids (siRNAs) synthesized as above, the present disclosure can further construct shRNA expression plasmids with the same or similar functions as the aforementioned nucleic acids. The methods for constructing such expression plasmids are well-known to those skilled in the art and will not be elaborated herein.

### Targeted Drug Delivery System

The present disclosure also provides a targeted drug delivery system, which comprises a targeting moiety, a linking moiety, and any of the aforementioned nucleic acids connected with the targeting moiety via the linking moiety.

In combination with common knowledge in the art, the nucleic acids (siRNAs) of the present disclosure exhibit superior inhibitory effects when applied to different targeted drug delivery systems. In other words, the advantageous effects of the naked sequences and modified sequences in the present disclosure do not depend on the choice of targeting vectors. To further improve the bioavailability and therapeutic efficacy of siRNAs, the present disclosure also optimizes the targeted drug delivery system and obtains the following technical solutions.

In some embodiments, the targeted drug delivery system comprises a ligand and the aforementioned nucleic acid linked to the ligand.

In some embodiments, the linking moiety (or ligand) is linked to the 5′ end or 3′ end of the sense strand or antisense strand of the nucleic acid. In some embodiments, the linking moiety (or ligand) is linked to the 5′ end of the sense strand or antisense strand of the nucleic acid. In some embodiments, the linking moiety (or ligand) is linked to the 3′ end of the sense strand or antisense strand of the nucleic acid.

In some embodiments, the linking moiety (or ligand) is linked to the 5′ end or 3′ end of the sense strand of the nucleic acid. In some embodiments, the linking moiety (or ligand) is linked to the 5′ end of the sense strand of the nucleic acid. In some embodiments, the linking moiety (or ligand) is linked to the 3′ end of the sense strand of the nucleic acid.

In some embodiments, the linking moiety (or ligand) is linked to the 5′ end or 3′ end of the antisense strand of the nucleic acid. In some embodiments, the linking moiety (or ligand) is linked to the 5′ end of the antisense strand of the nucleic acid. In some embodiments, the linking moiety (or ligand) is linked to the 3′ end of the antisense strand of the nucleic acid.

In some embodiments, the linking moiety (or ligand) is linked to the 3′ end of the sense strand of the nucleic acid.

In some embodiments, the ligand comprises a structure shown in formula (XI) or formula (XII):

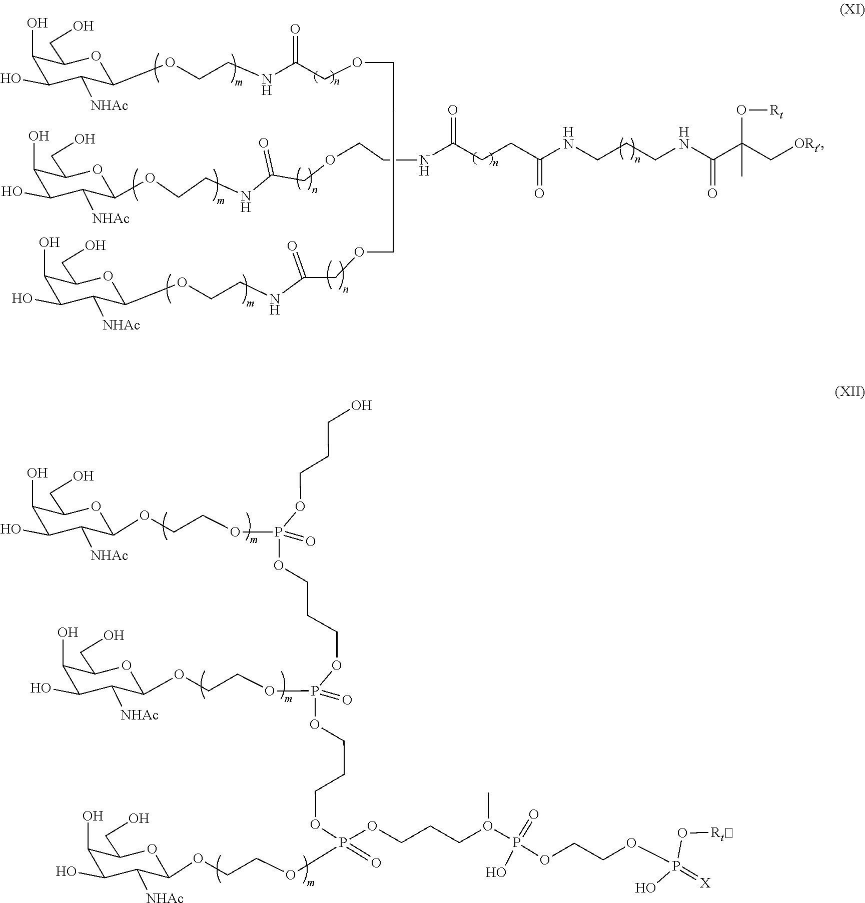

wherein m and n in formula (XI) or formula (XII) are each independently an integer of 0-6; Rₜ and R_{t′} are each independently any of the aforementioned nucleic acids.

In some embodiments, the targeted drug delivery system has a structure shown in formula (601), (602), (603), (604), (605), (606), or (607):

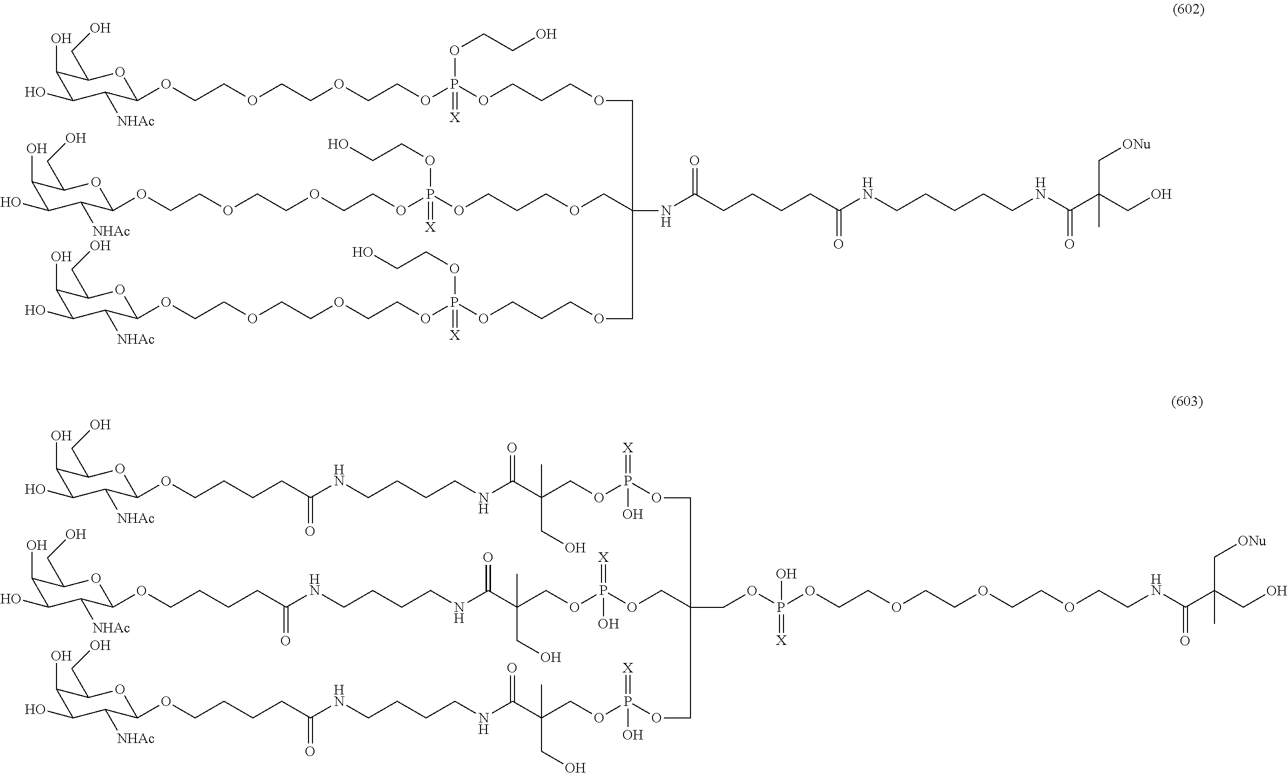

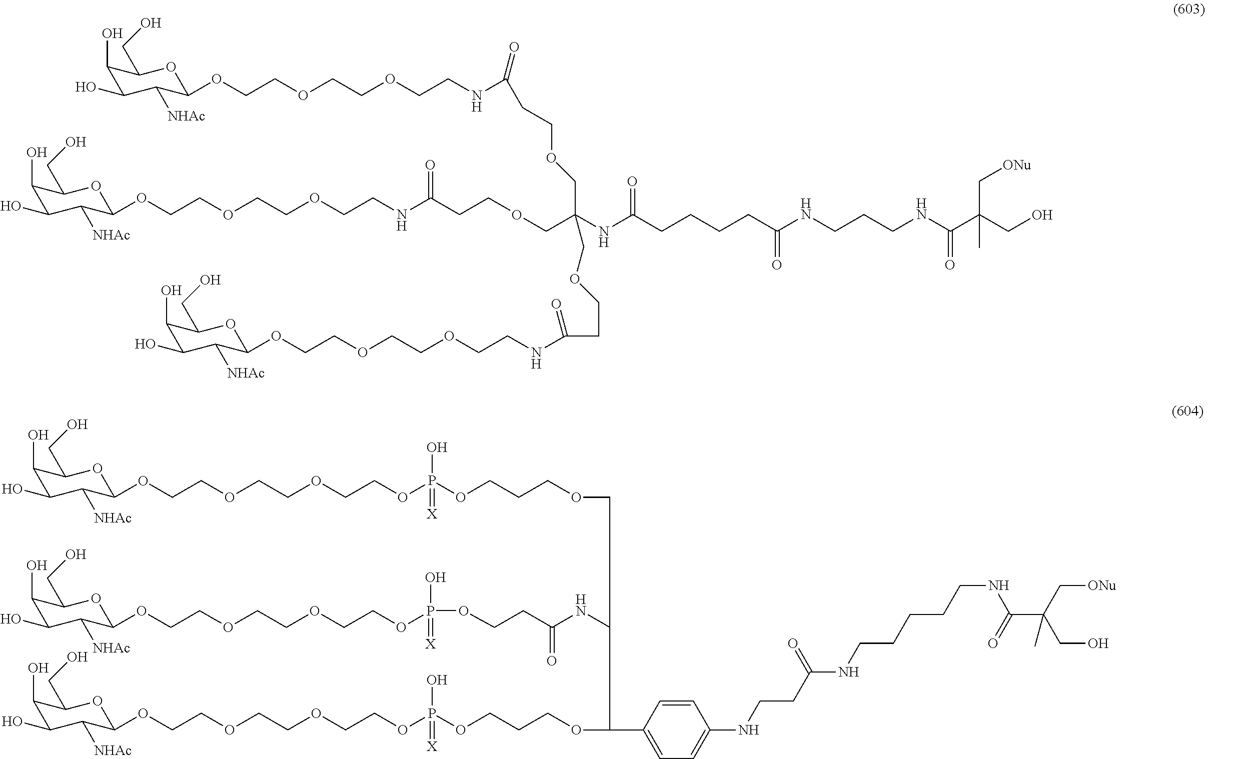

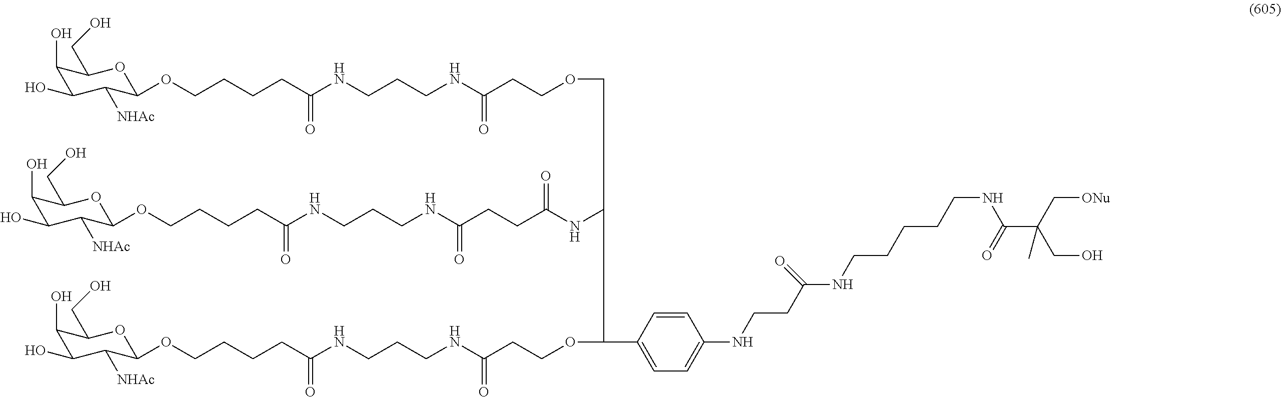

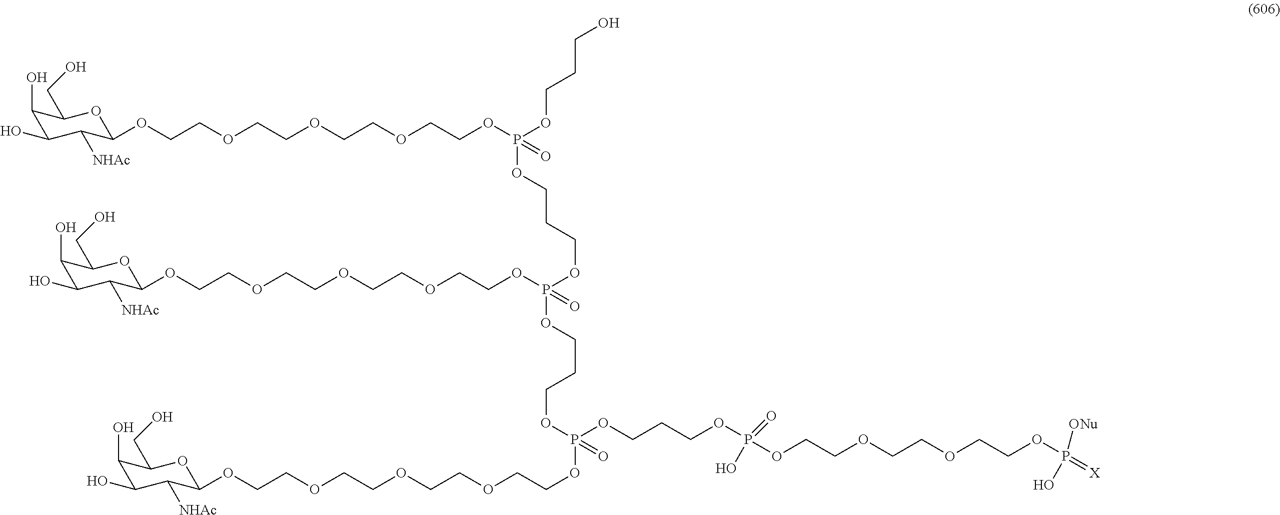

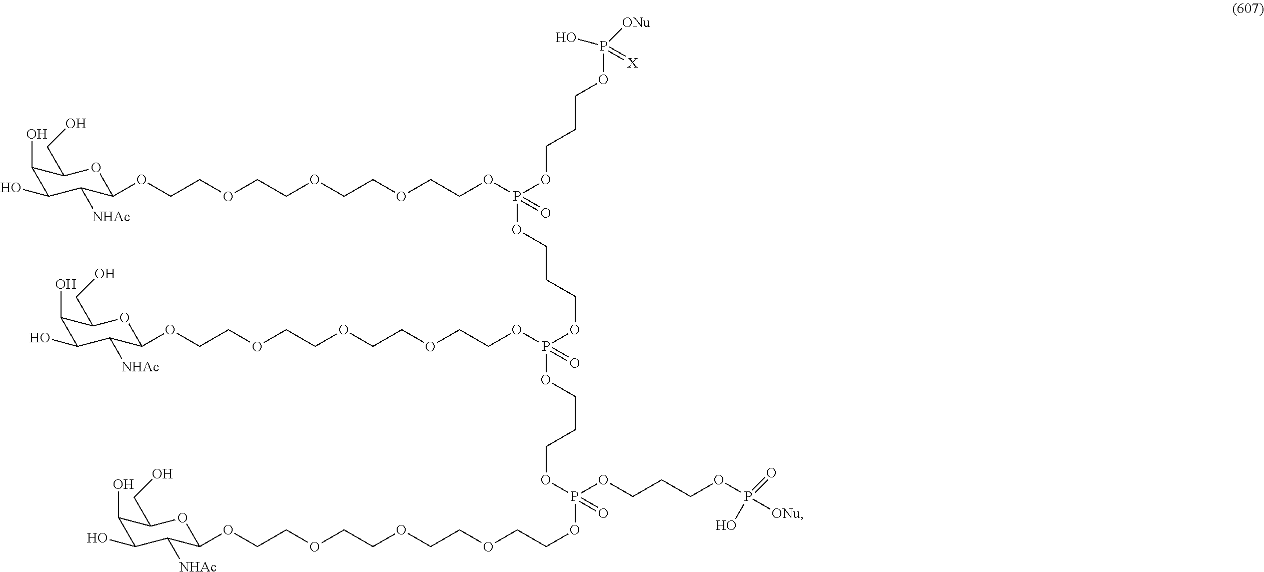

wherein X in formula (601), (602), (603), (604), (605), (606), or (607) is O or S; and Nu is any of the aforementioned nucleic acids.

In the present disclosure, the conjugate having the structure shown in formula (603) can be obtained by the following method: contacting the compound shown in formula (603A) with an anhydride (e.g., succinic anhydride) to obtain the corresponding carboxylate (603B), further connecting the carboxylate (603B) to a solid phase (SPS) to obtain compound (603C), and further connecting the compound (603C) to an active group Nu (Nu=the nucleic acid according to any aspect of the present disclosure).

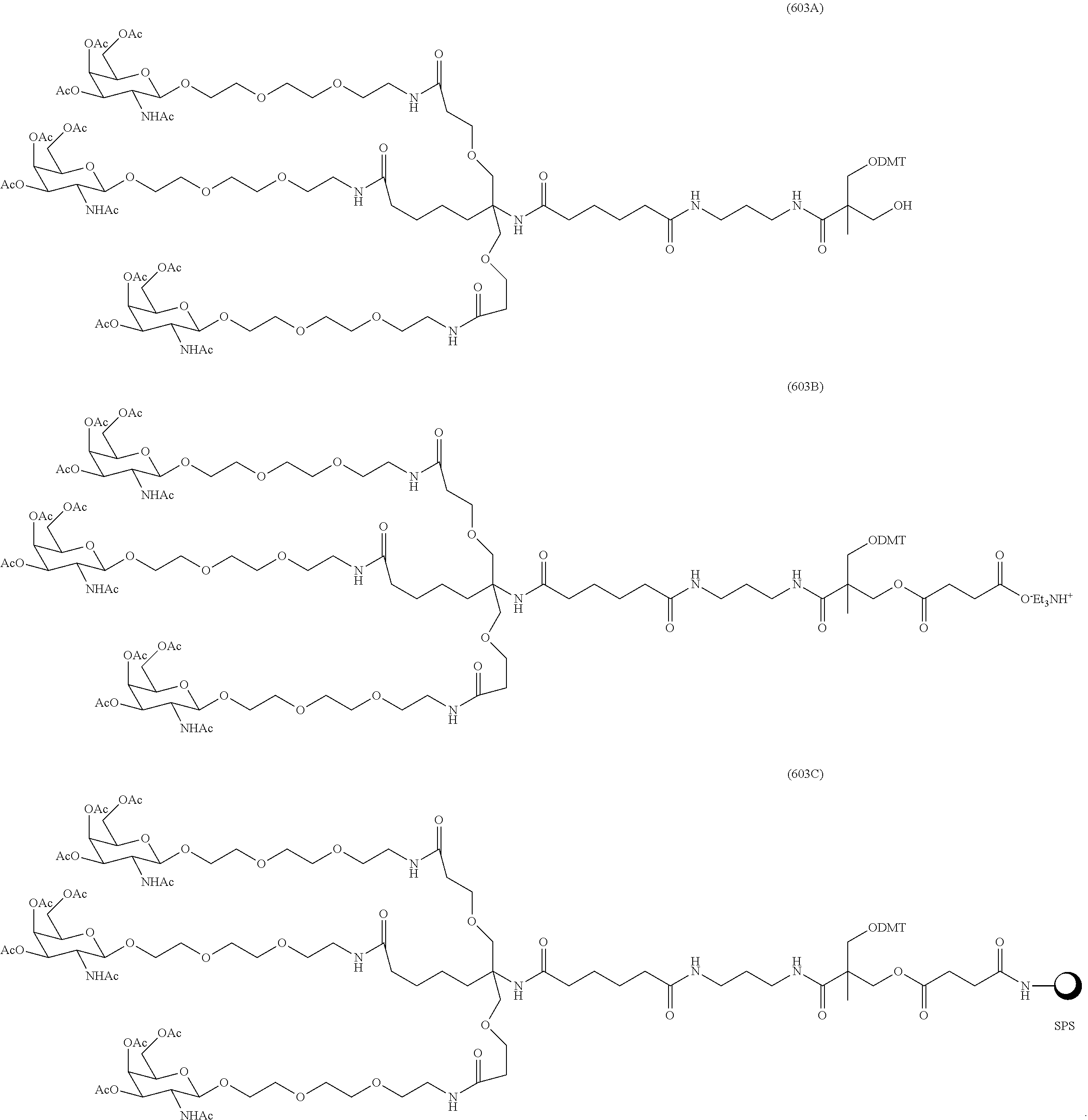

In some preferred embodiments, the targeted drug delivery system has a structure shown in formula (603) or formula (606).

In some embodiments the targeted drug delivery system comprises a ligand and the nucleic acid linked to the ligand, and the ligand has a structure shown formula (I), (II) or (V):

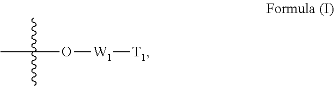

wherein W₁ is selected from a direct linkage or

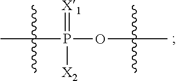

wherein X′₁ is selected from an O or S atom, or is absent;
when X′₁ is selected from an O or S atom, X₂ is selected from —O—, —S—, —SH, —OH (hydroxyl), —NH₂ (amino), C₁-C₆ alkyl, C₁-C₆ alkoxy, or —O—(CH₂)_{n′}—OR′₅, R′₅ is selected from H, a direct linkage or

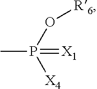

wherein R′₆ is H or a direct linkage, X₁ is selected from an O or S atom, X₄ is —OH or —SH, and n′ is an integer from 1 to 10; when X′₁ is absent, X₂ is a direct linkage;
T₁ is selected from —(CH₂)ₘCH₃, wherein m is an integer from 10 to 30; or

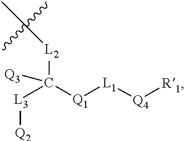

wherein Q₁ and Q₄ are each independently selected from a direct linkage, —NH₂ (amino), —COOH (carboxyl), amide group (—NHCO— or —CONH—), —O—, —S—, —S—S—, phosphate ester group, or phosphorothioate group;
Q₂ is selected from —SH, —OH (hydroxyl), —NH₂ (amino), —H, C₁-C₆ alkyl, preferably —CH₃ (methyl), —COOH (carboxyl), amide group (—NHCO— or —CONH—), —O—, —S—, —S—S—, phosphate ester group, phosphorothioate group, or

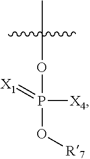

wherein R′₇ is H or a direct linkage, and X₁ and X₄ are as defined above;
Q₃ is selected from —H or C₁-C₁₀ alkyl;
L₁ is —(CH₂)ₗ—(NR′₄)ₜ—(CH₂)_{q}—, wherein l and q are integers from 0 to 10, and l+q=1-10, t is 0 or 1, and R′₄ is —CO(CH₂)ᵣCOOH, wherein r is an integer from 10 to 30;
L₂ and L₃ are each independently selected from a C₁-C₁₀ saturated alkane chain or a direct linkage;
R′₁ is selected from a C₁₀-C₃₀ saturated fatty acid chain, a C₁₀-C₃₀ saturated alkane chain, a C₁₀-C₃₀ unsaturated hydrocarbon group, or —(CH₂)ₘ—X₃—R′₃, wherein m is an integer from 10 to 30, X₃ is selected from a direct linkage, an oxygen atom or a sulfur atom, R′₃ is selected from a nitrogen- and oxygen-containing saturated six-membered heterocyclic group, H, a direct linkage, or

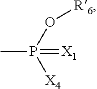

wherein R′₆, X₁ and X₄ are as defined above; when X₃ is a direct linkage, R′₃ is neither H nor a direct linkage;
when W₁ is a direct linkage, T₁ is not —(CH₂)ₘCH₃;

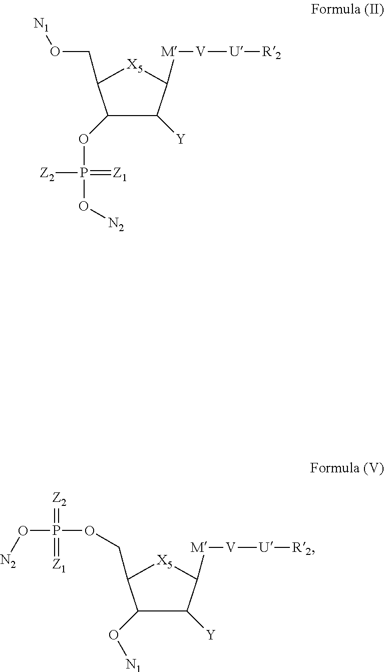

in formulae (II) and (V), the five-membered ring is a five-membered cyclic sugar structure in ribose or deoxyribose, wherein X₅ is selected from —CH₂—, —CH(CH₃)—, —C(CH₃)₂—, —O—, —NH—, —N(CH₃)— or —S—;
M′ is selected from H, —O—, —C— or a modified or unmodified nucleotide base;
N₁ is selected from a direct linkage, H, C₁-C₃ alkyl or

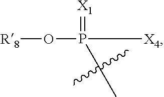

wherein R′₈ is H or a direct linkage, and X₁ and X₄ are as defined above;
N₂ is selected from a direct linkage, H or C₁-C₃ alkyl;
Y is selected from H, NH₂, OH, halogen, C₁-C₆ alkyl, C₁-C₆ haloalkyl, —O—R′₉ or —O—(CH₂)n-O—R′₁₀, wherein R′₉ is C₁-C₆ alkyl, preferably —O—CH₃, n is an integer from 1 to 6, R′₁₀ is C₁-C₆ alkyl, preferably n is 2, and R′₁₀ is C₁ alkyl, i.e., 2′-methoxyethoxy;
V is selected from a C₁-C₄ saturated alkane chain or is absent;
U′ is selected from —NH₂ (amino), —COOH (carboxyl), amide group (—NHCO— or —CONH—), or is absent;
Z₁ is selected from an O or S atom;
Z₂ is selected from C₁₀-C₃₀ alkoxy, fatty acid chain (preferably terminal carboxyl fatty acid), amide lipid chain, alkene chain, or alkane chain;
R′₂ is selected from C₁₀-C₃₀ alkoxy, fatty acid chain (preferably terminal carboxyl fatty acid), amide lipid chain, alkene chain, alkane chain or is absent.

In some embodiments, the ligand is selected from at least one of the following structures (L1)-(L36) and (L′10):

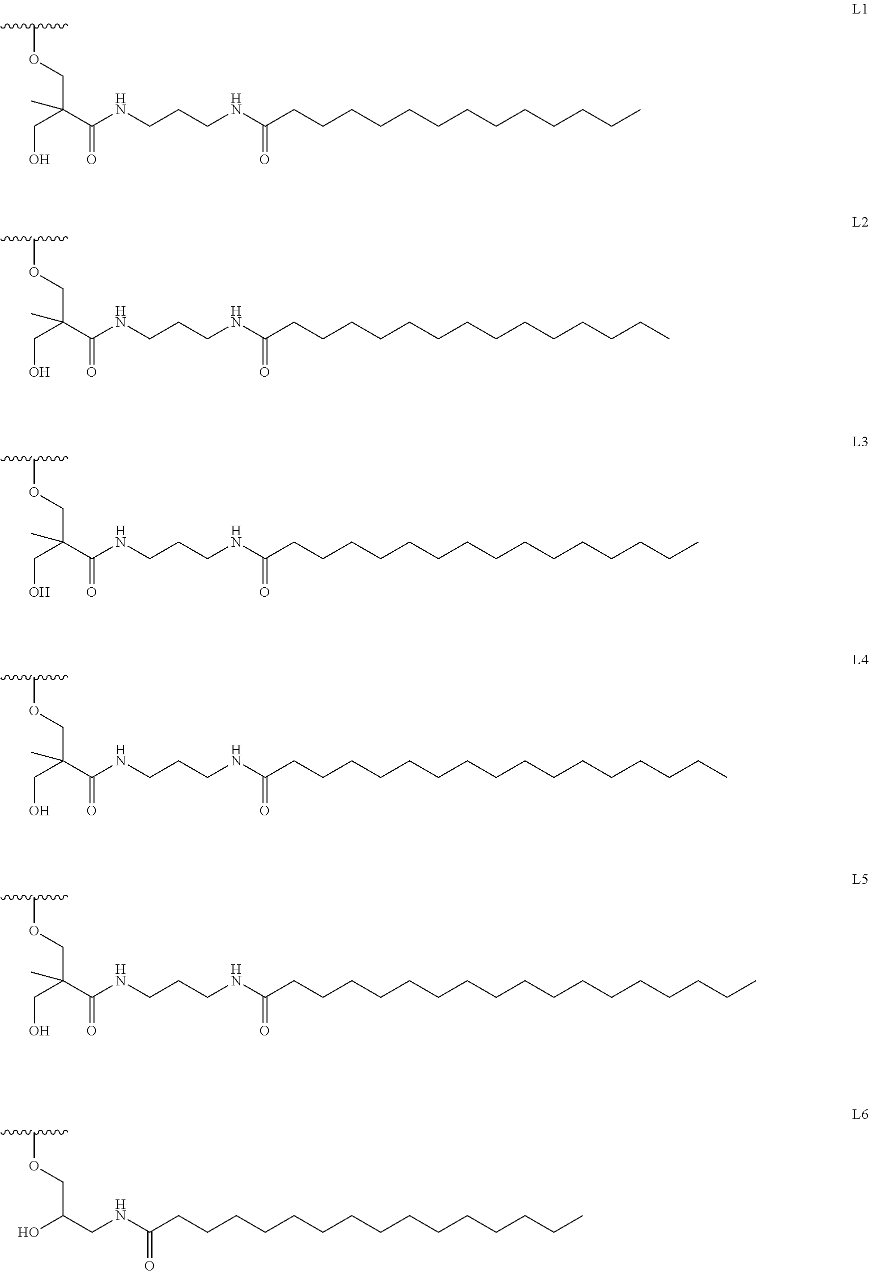

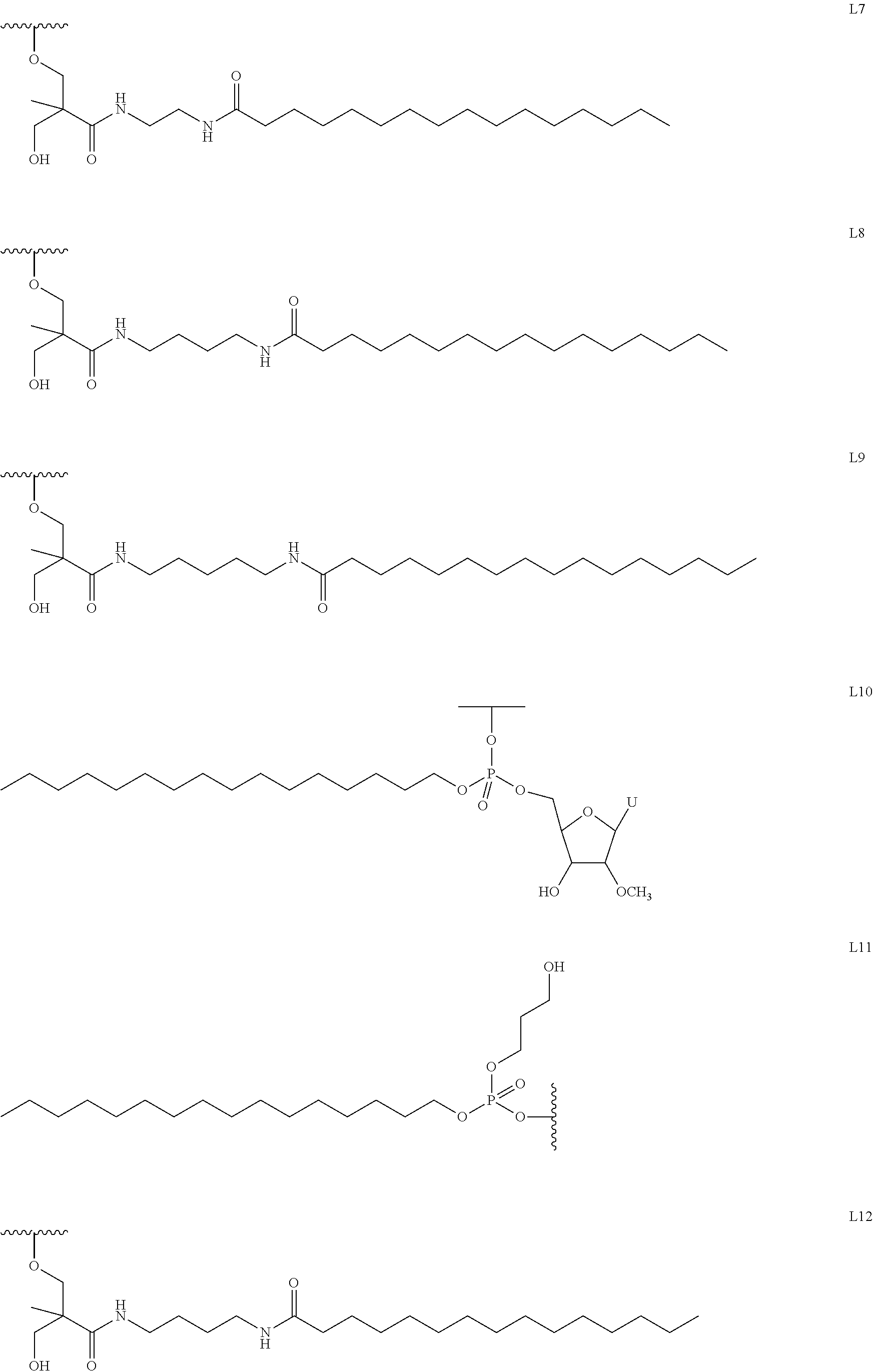

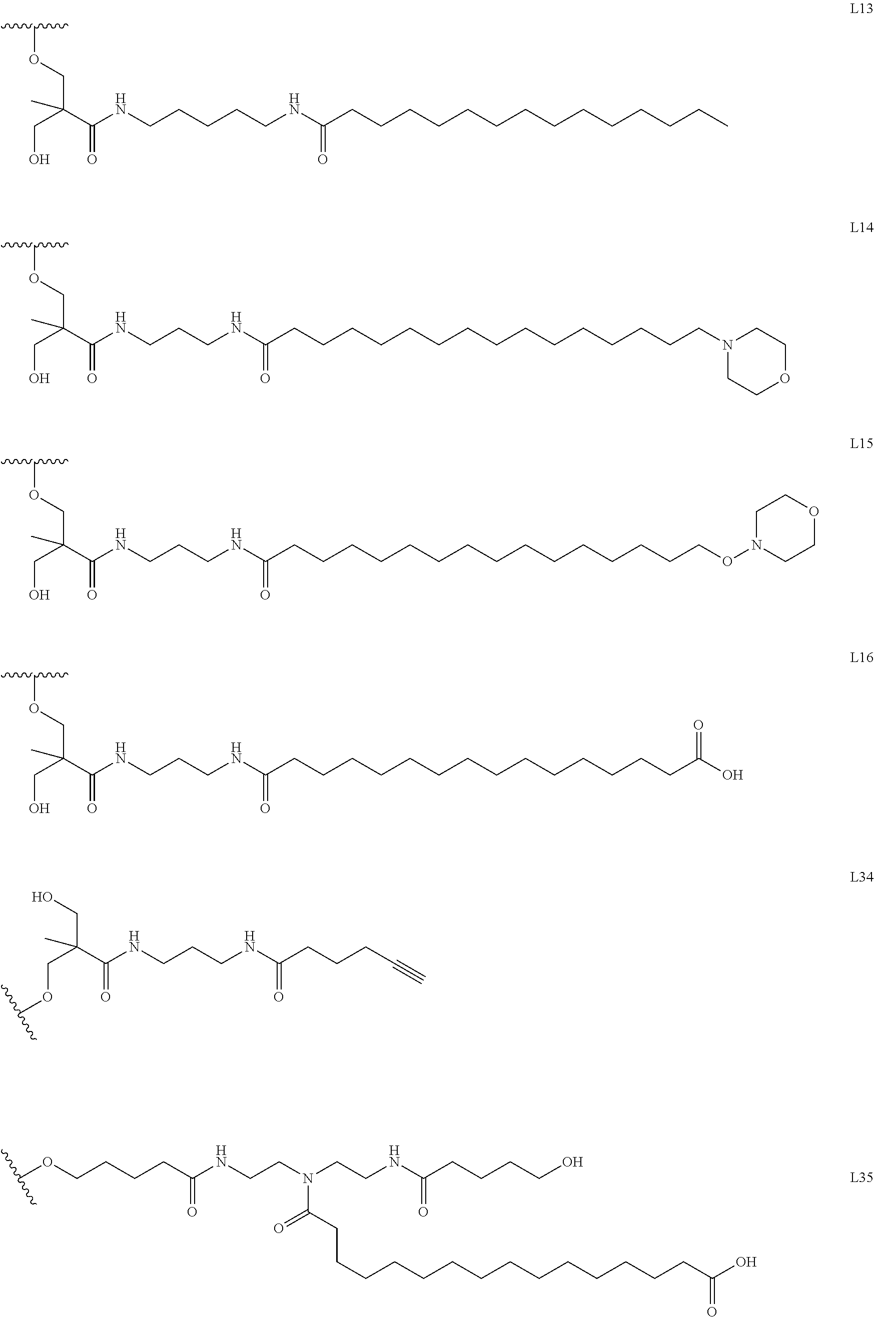

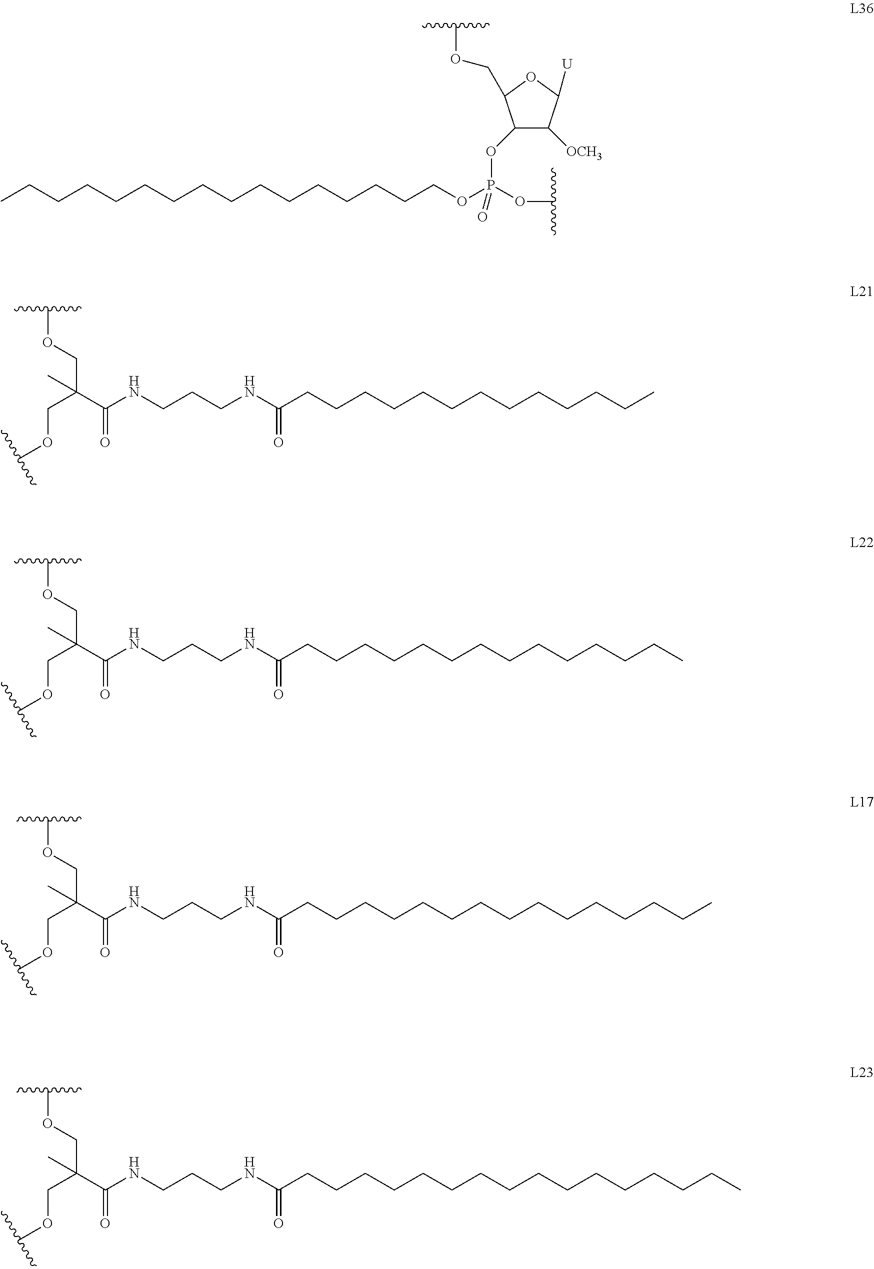

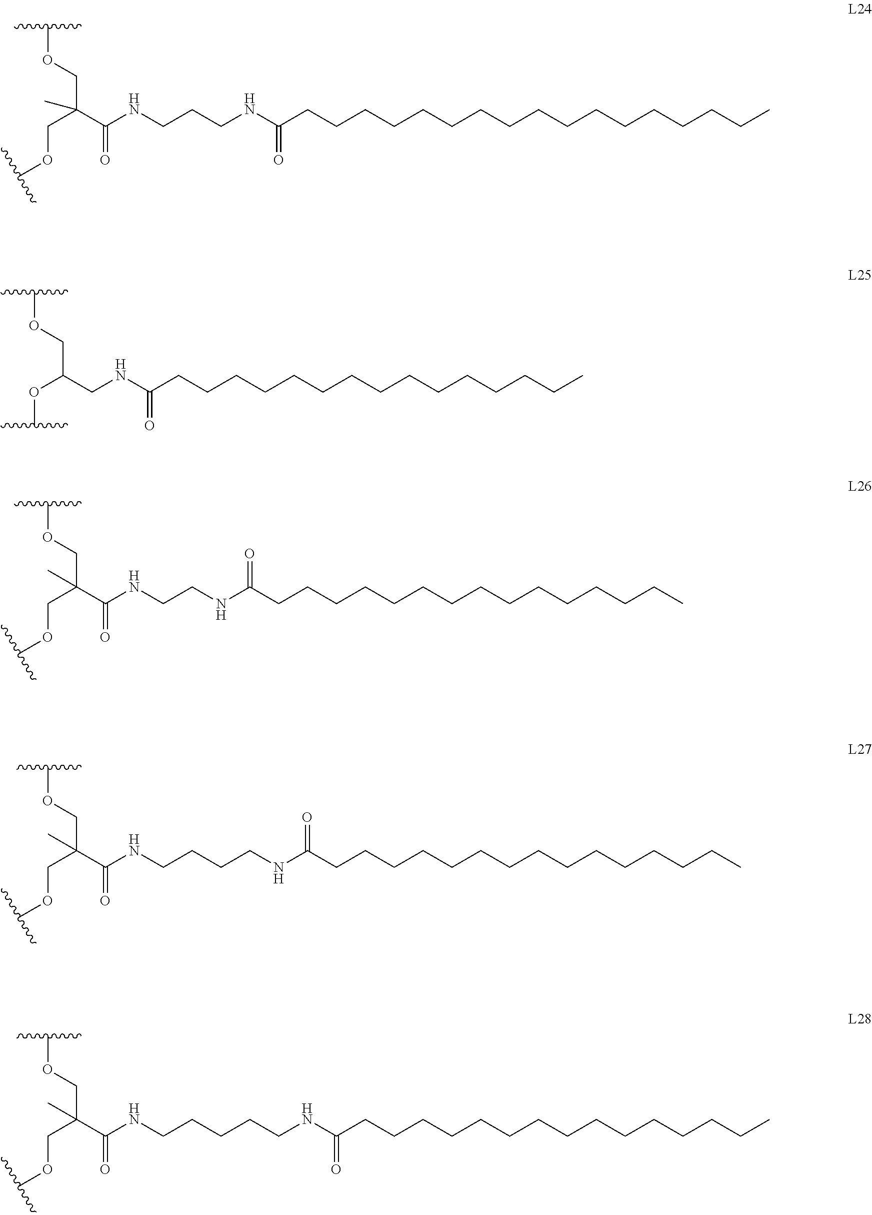 [SMILES: *OCC(C)(CO*)C(=O)NCCCNC(=O)CCCCCCCCCCCCCCCCC ; *OCC(CNC(=O)CCCCCCCCCCCCCCC)O* ; *OCC(C)(CO*)C(=O)NCCNC(=O)CCCCCCCCCCCCCCC ; *OCC(C)(CO*)C(=O)NCCCCNC(=O)CCCCCCCCCCCCCCC ; *OCC(C)(CO*)C(=O)NCCCCCNC(=O)CCCCCCCCCCCCCCC]

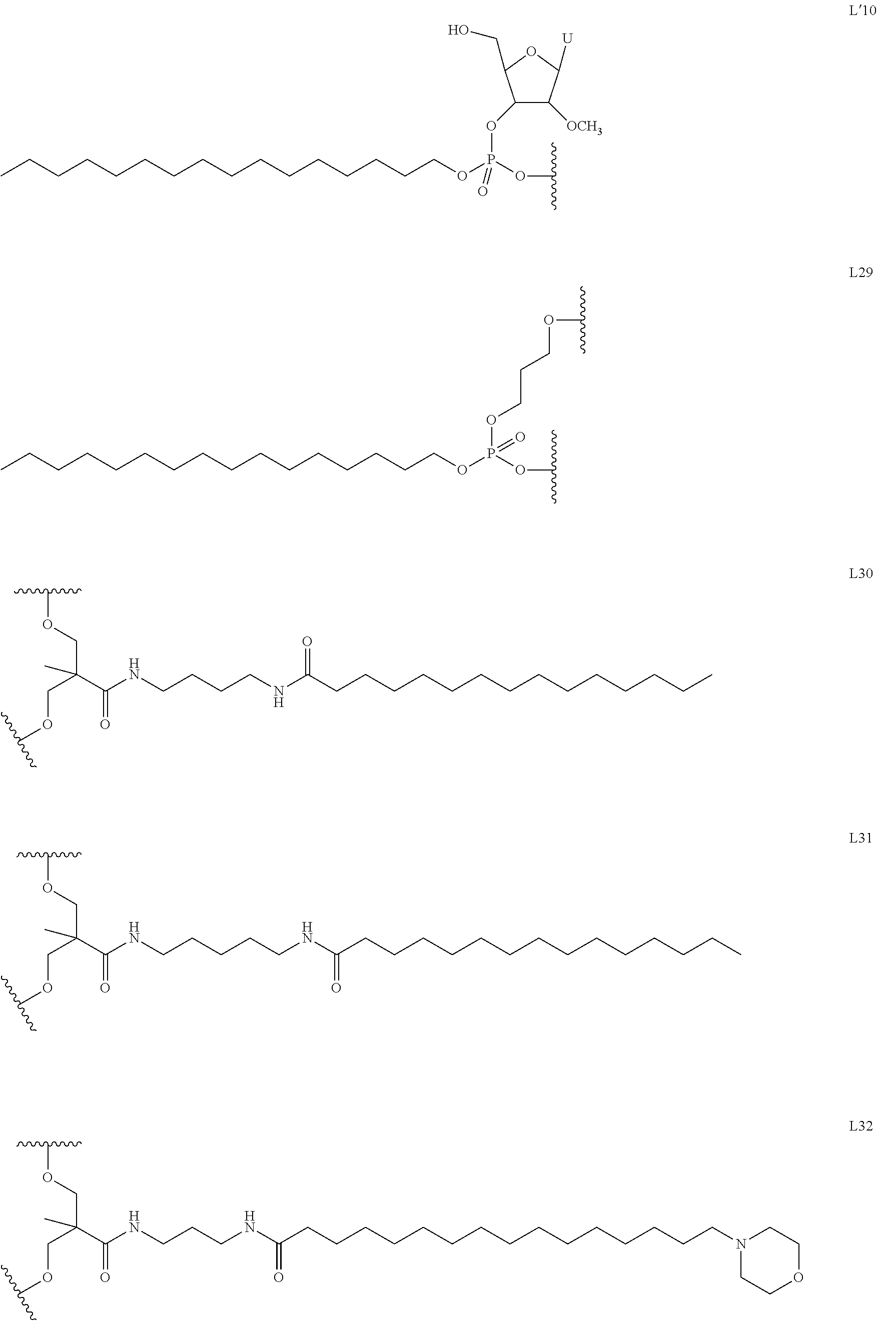

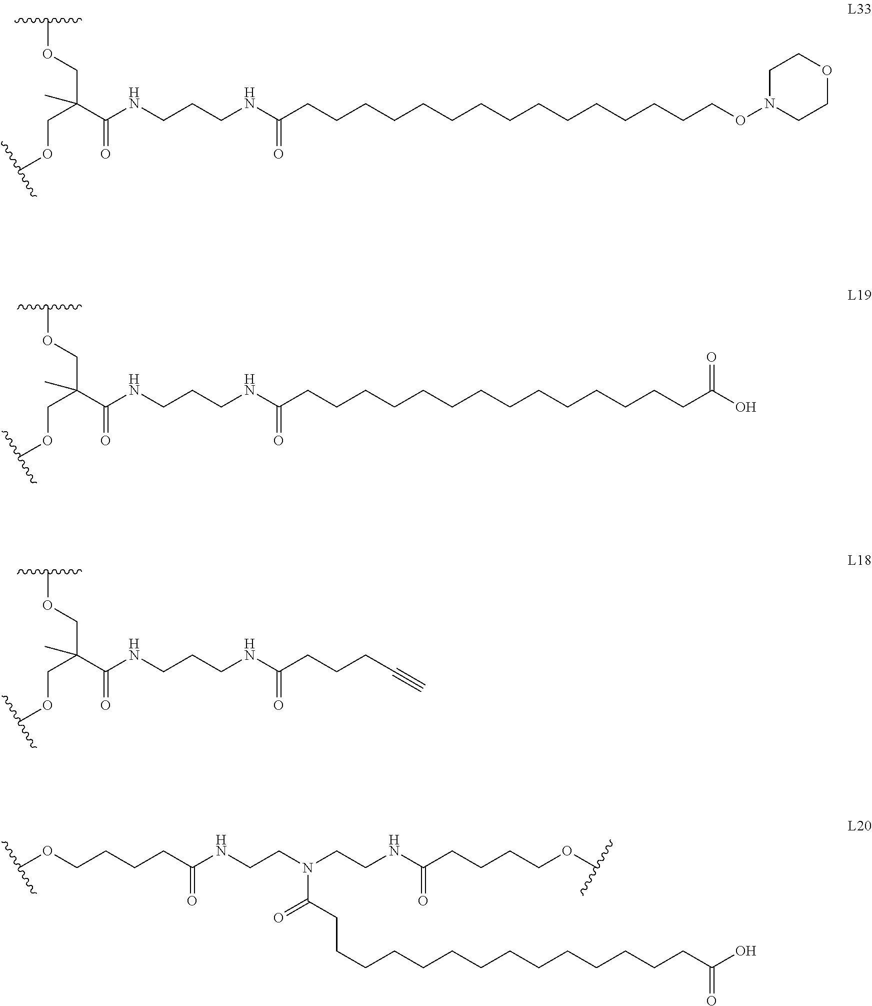 [SMILES: *OCC(C)(CO*)C(=O)NCCCNC(=O)CCCCCCCCCCCCCCCON1CCOCC1 ; *OCC(C)(CO*)C(=O)NCCCNC(=O)CCCCCCCCCCCCCCC(=O)O ; *OCC(C)(CO*)C(=O)NCCCNC(=O)CCCC#C ; *OCCCCC(=O)NCCN(CCNC(=O)CCCCO*)C(=O)CCCCCCCCCCCCCCC(=O)O]

wherein U

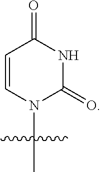 [SMILES: *n1ccc(=O)[nH]c1=O]

In some embodiments, the ligand is selected from at least one of the following structures (L1′)-(L36′) and (L′10′):

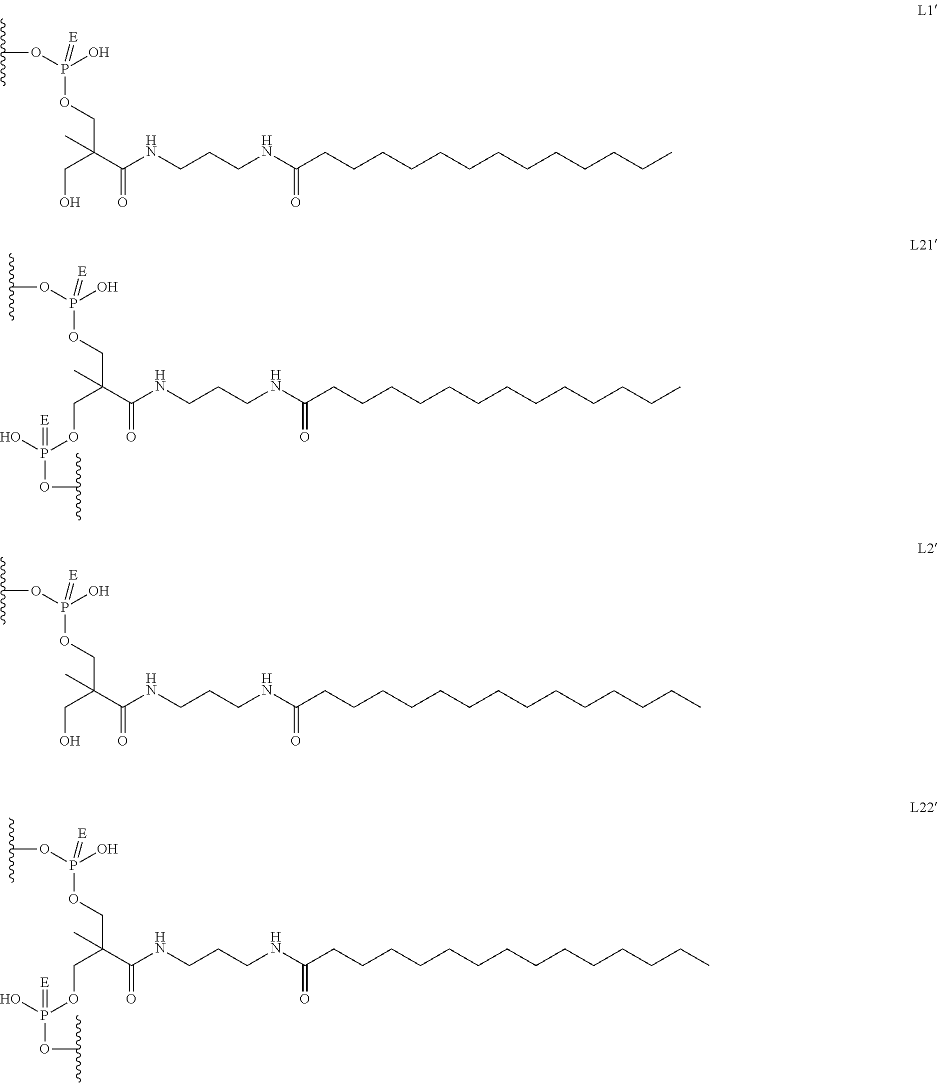

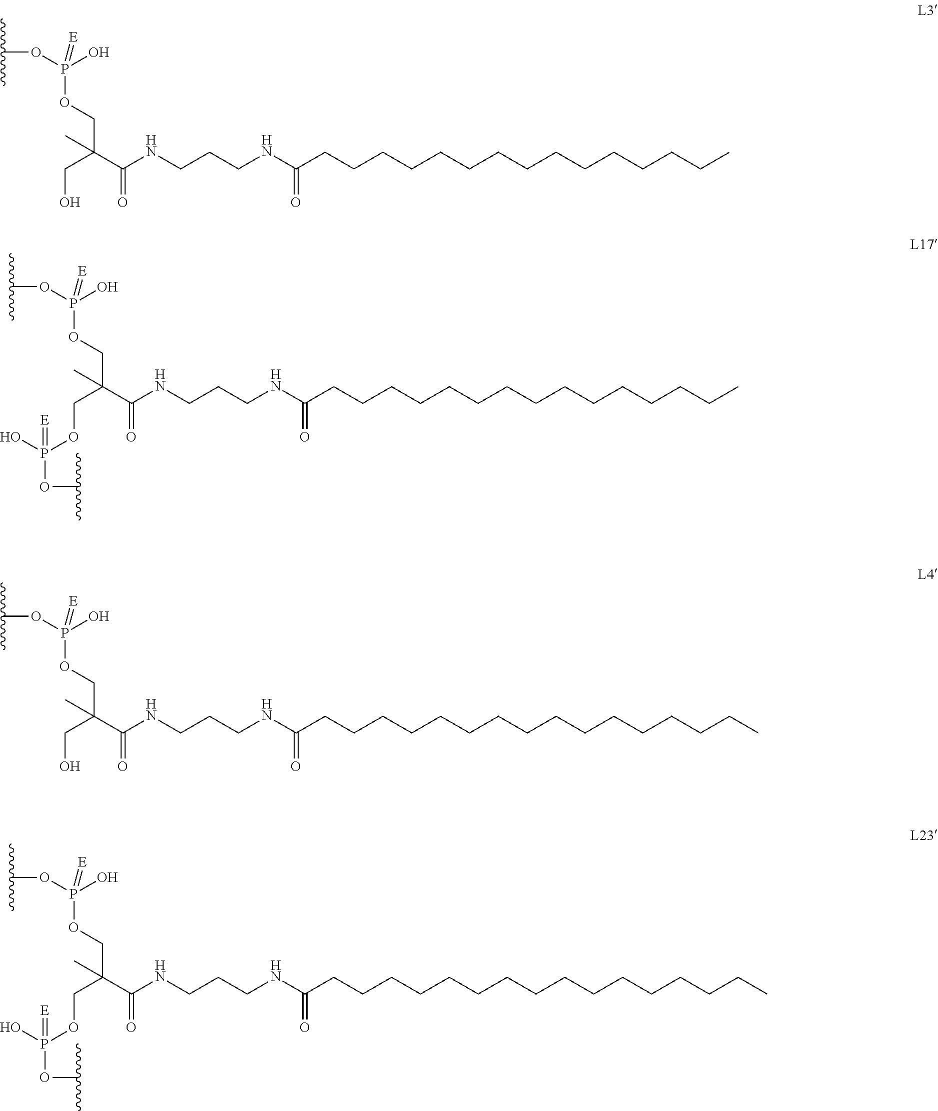

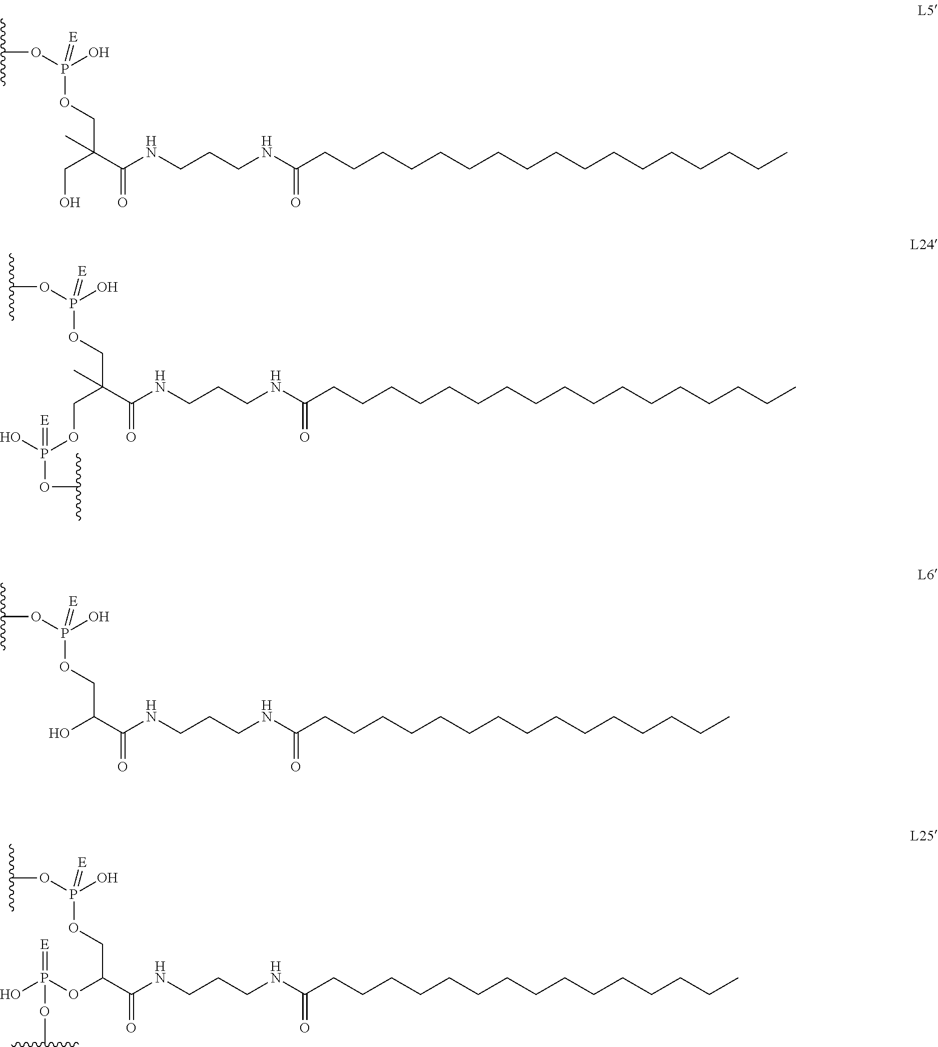

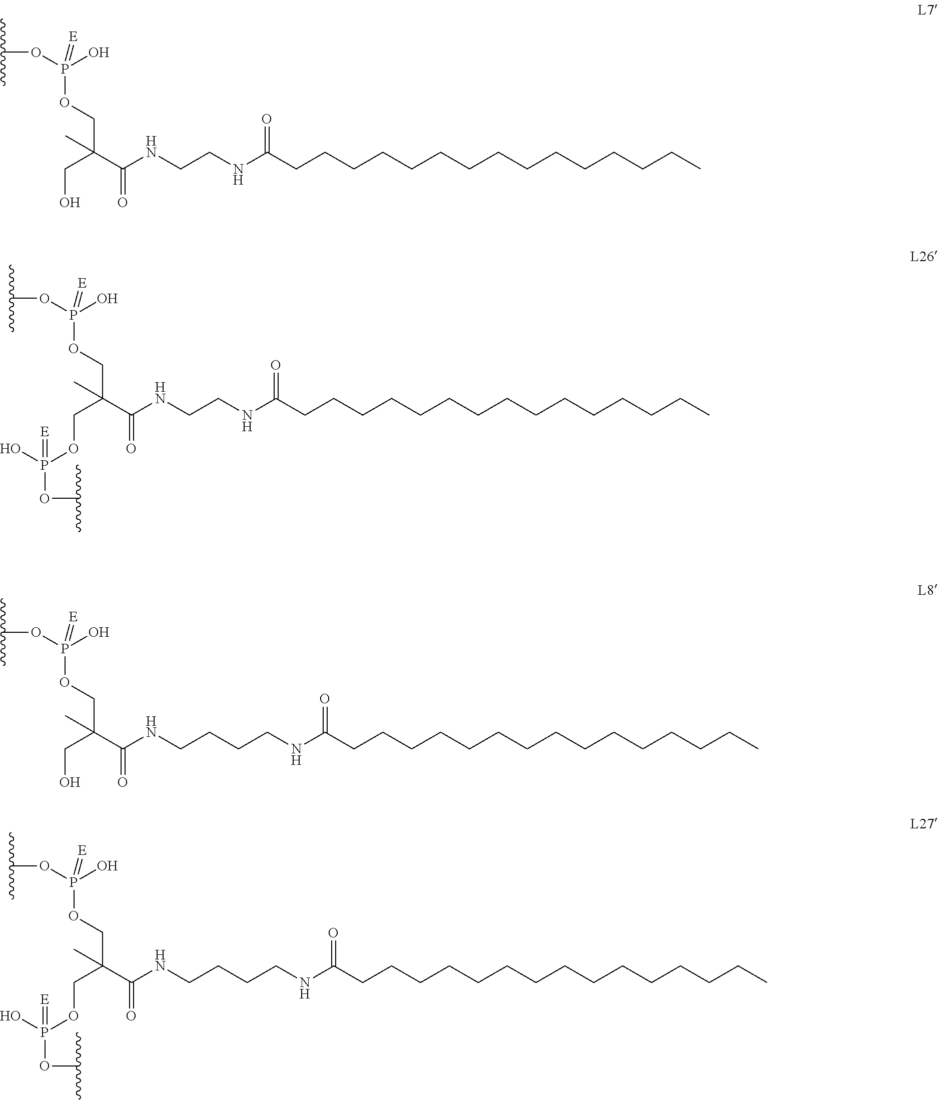

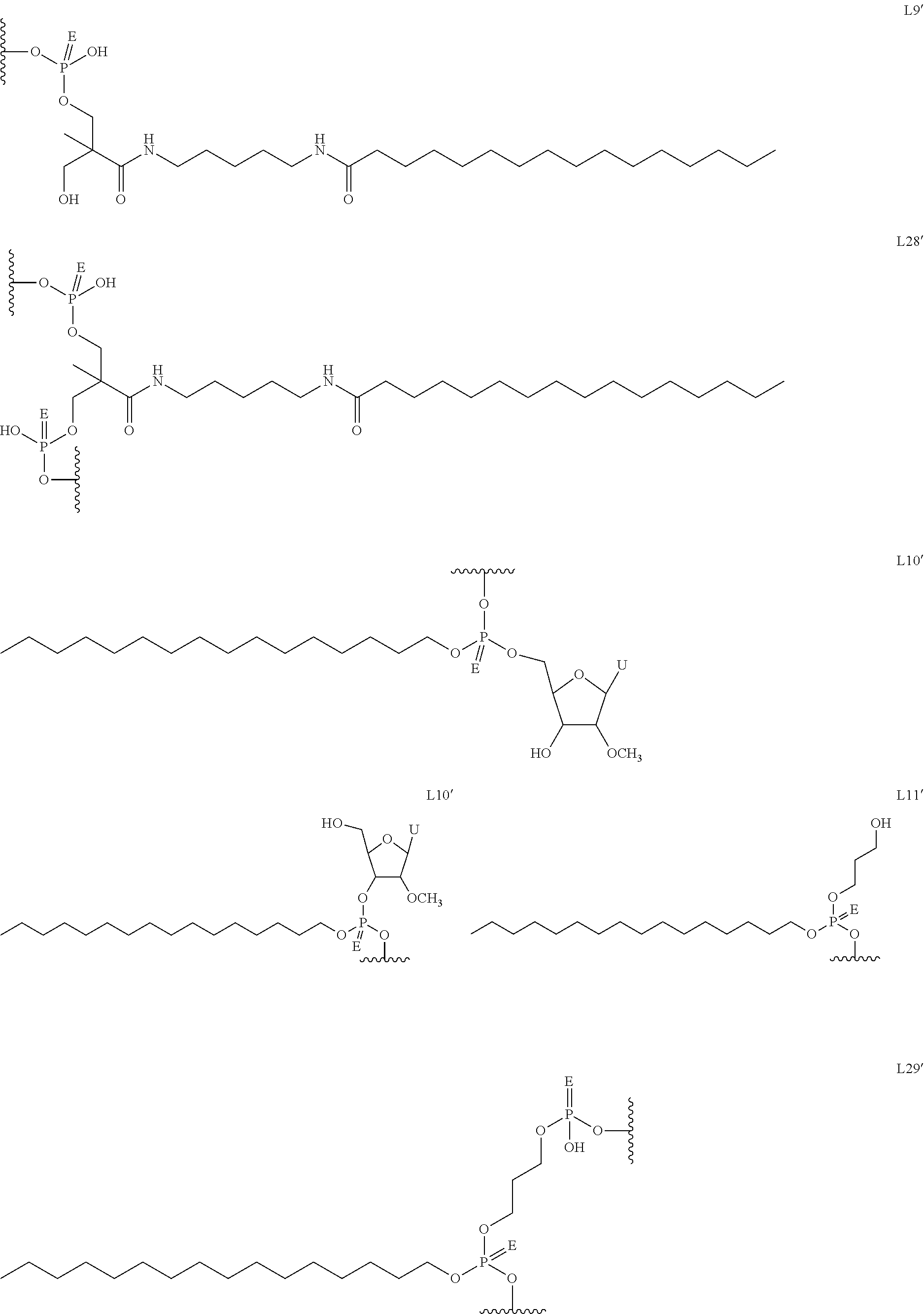

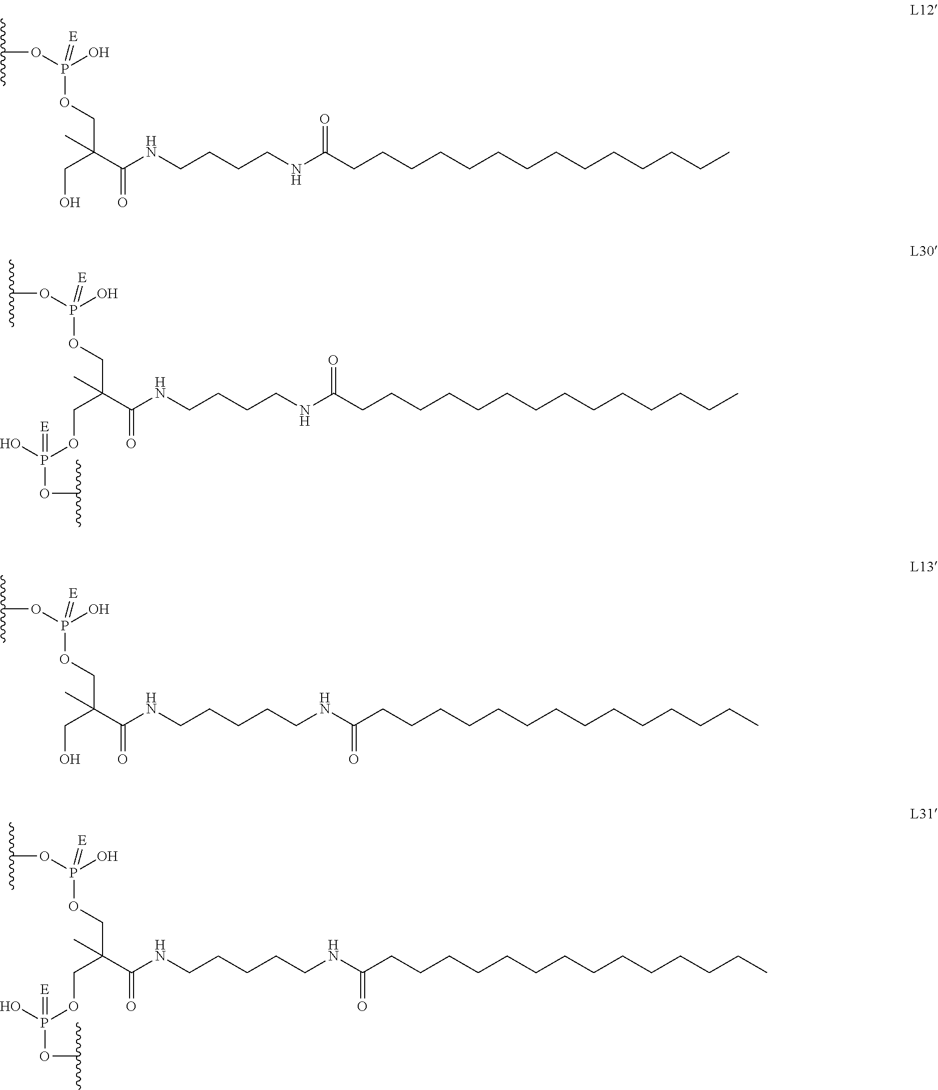

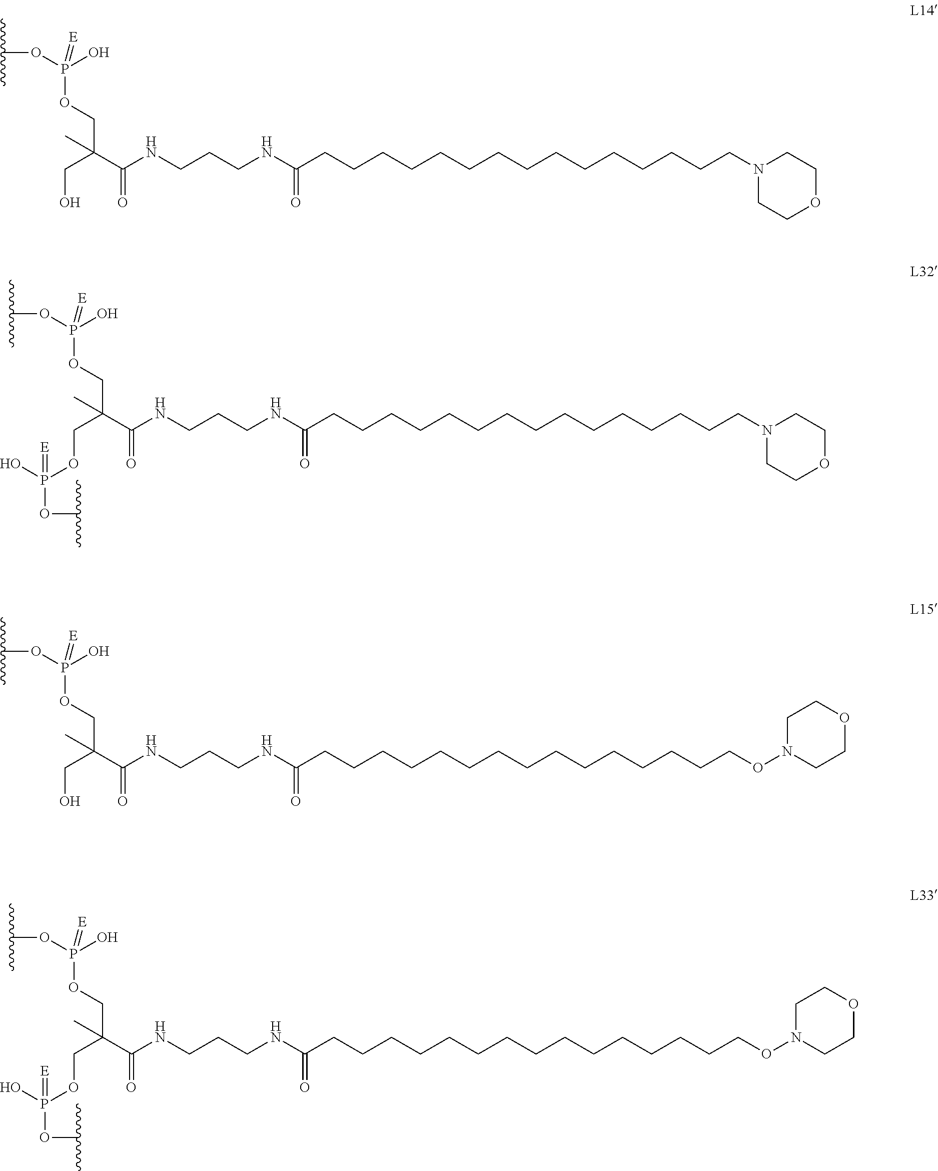

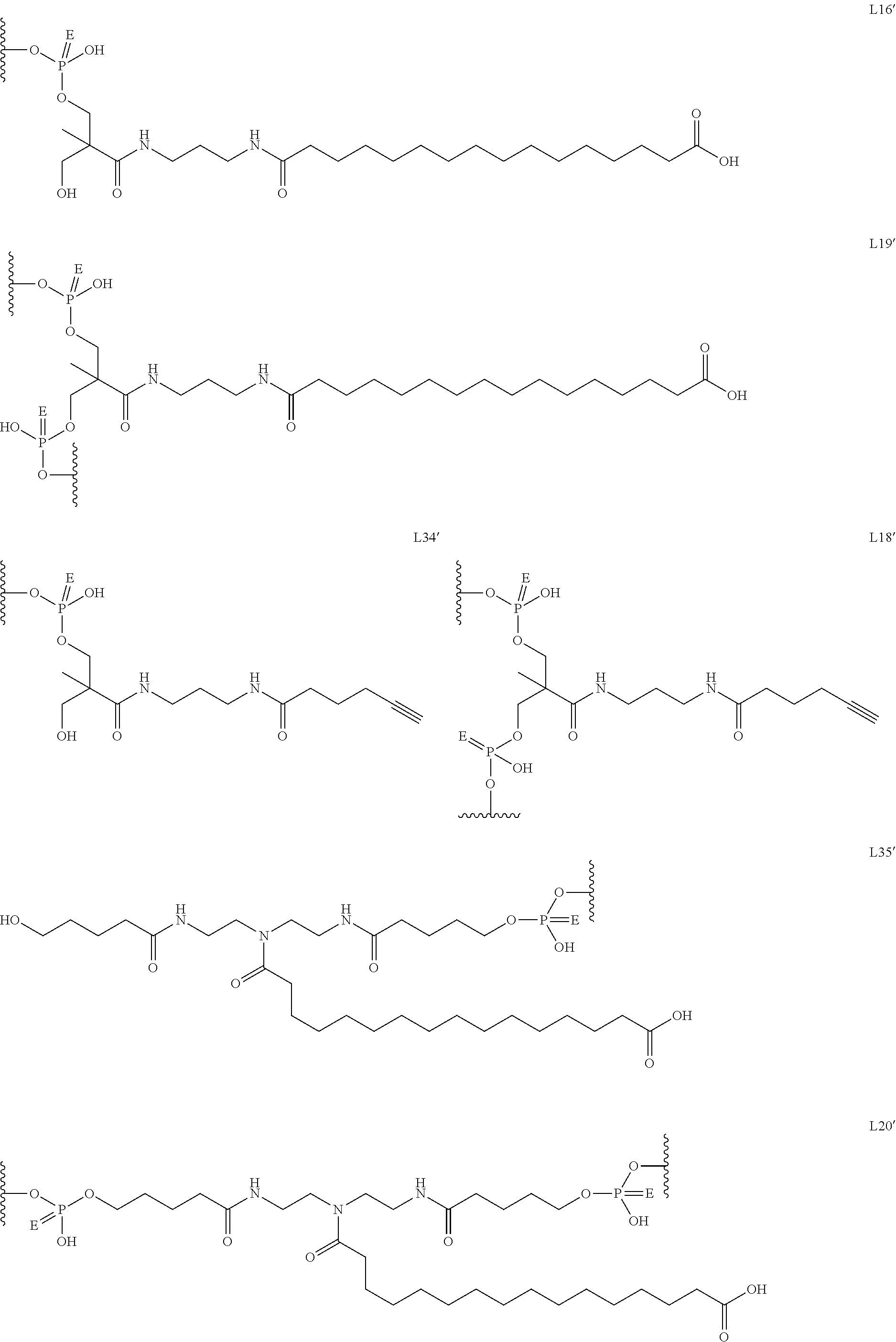

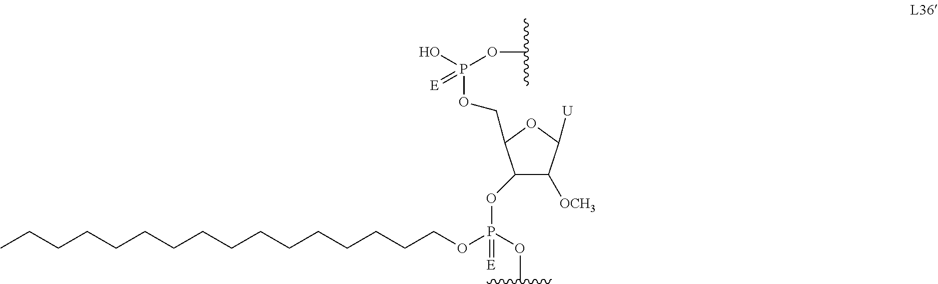

wherein E is selected from O and S, and U is

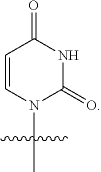 [SMILES: *n1ccc(=O)[nH]c1=O]

In some embodiments, the targeted drug delivery system comprises a structure shown in Formula (III), Formula (IV), or Formula (VI).

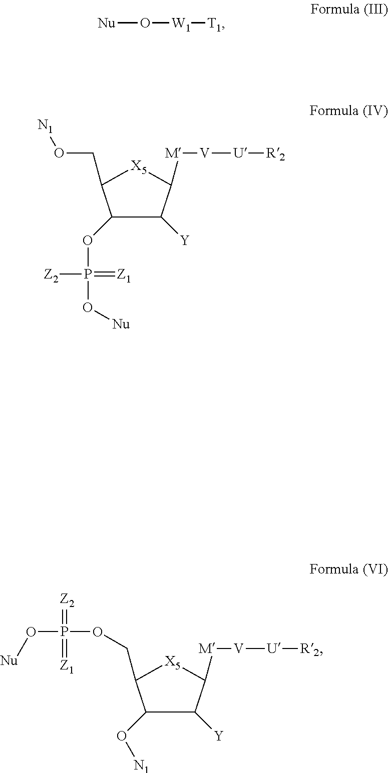

wherein Nu is a nucleic acid, and other variables are as previously defined.

In some preferred embodiments, the targeted drug delivery system has a structure of formula (III), wherein
1) X₂ is —O—(CH₂)_{n′}—OR′₅, wherein R′₅ is selected from a direct linkage or

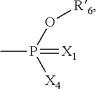

wherein R′₆ is a direct linkage, and n′ is an integer from 1 to 10;
2) Q₂ is selected from —O—, —S— or

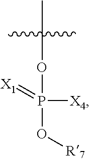

wherein R′₇ is selected from a direct linkage; or
3) R′₁ is selected from —(CH₂)ₘ—X₃—R′₃, wherein m is an integer from 10 to 30, X₃ is selected from an oxygen atom, a sulfur atom, a direct linkage, or

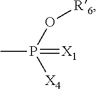

wherein R′₆ is a direct linkage;

In some preferred embodiments, the targeted drug delivery system has the structure of formula (IV) or formula (VI), wherein N₁ is a direct linkage or

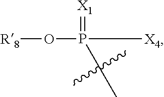

wherein R′₈ is selected from a direct linkage.

In some preferred embodiments, the targeted drug delivery system is selected from at least one of the following structures (L1″)-(L36″) and (L′10″):

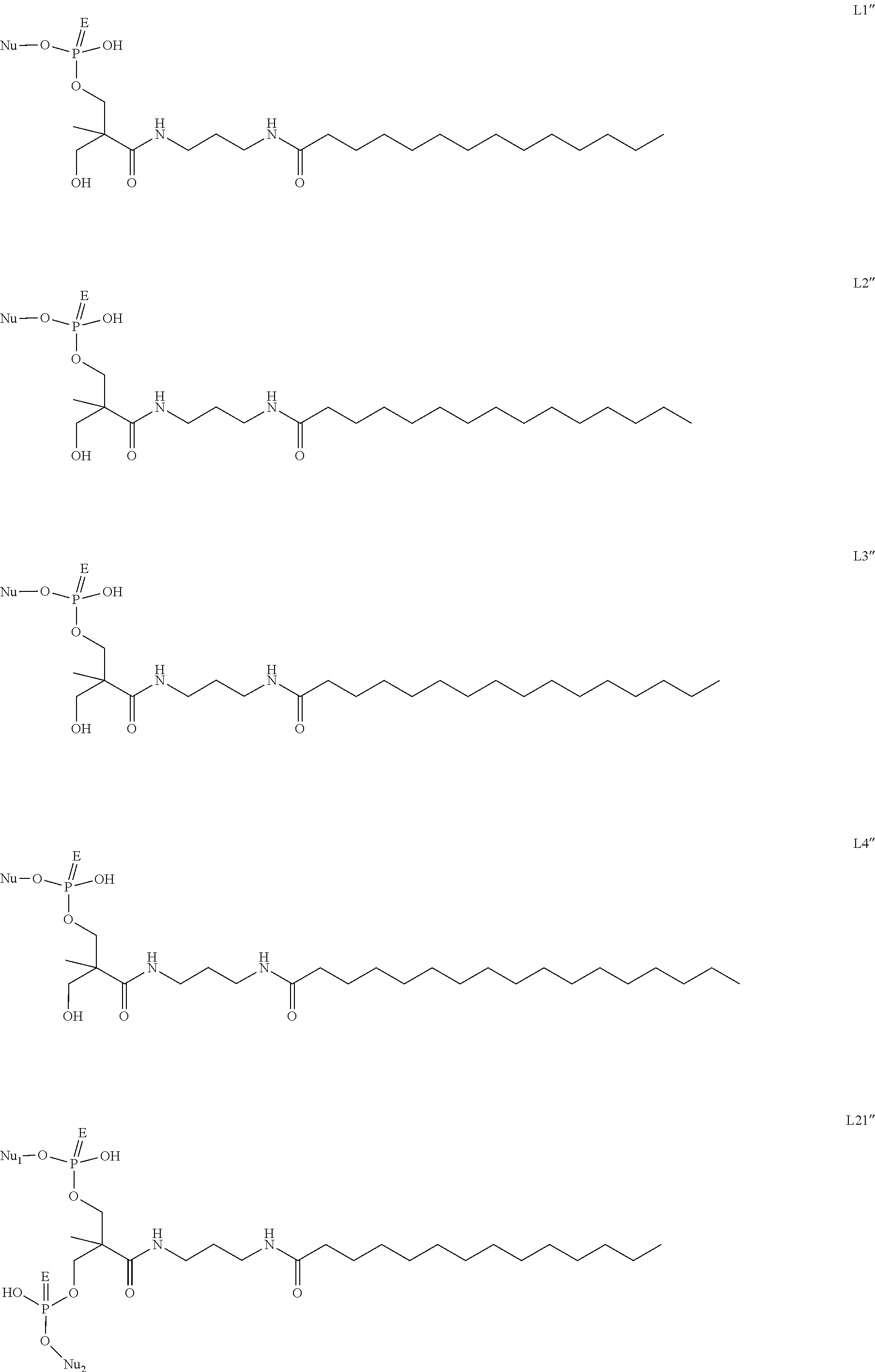

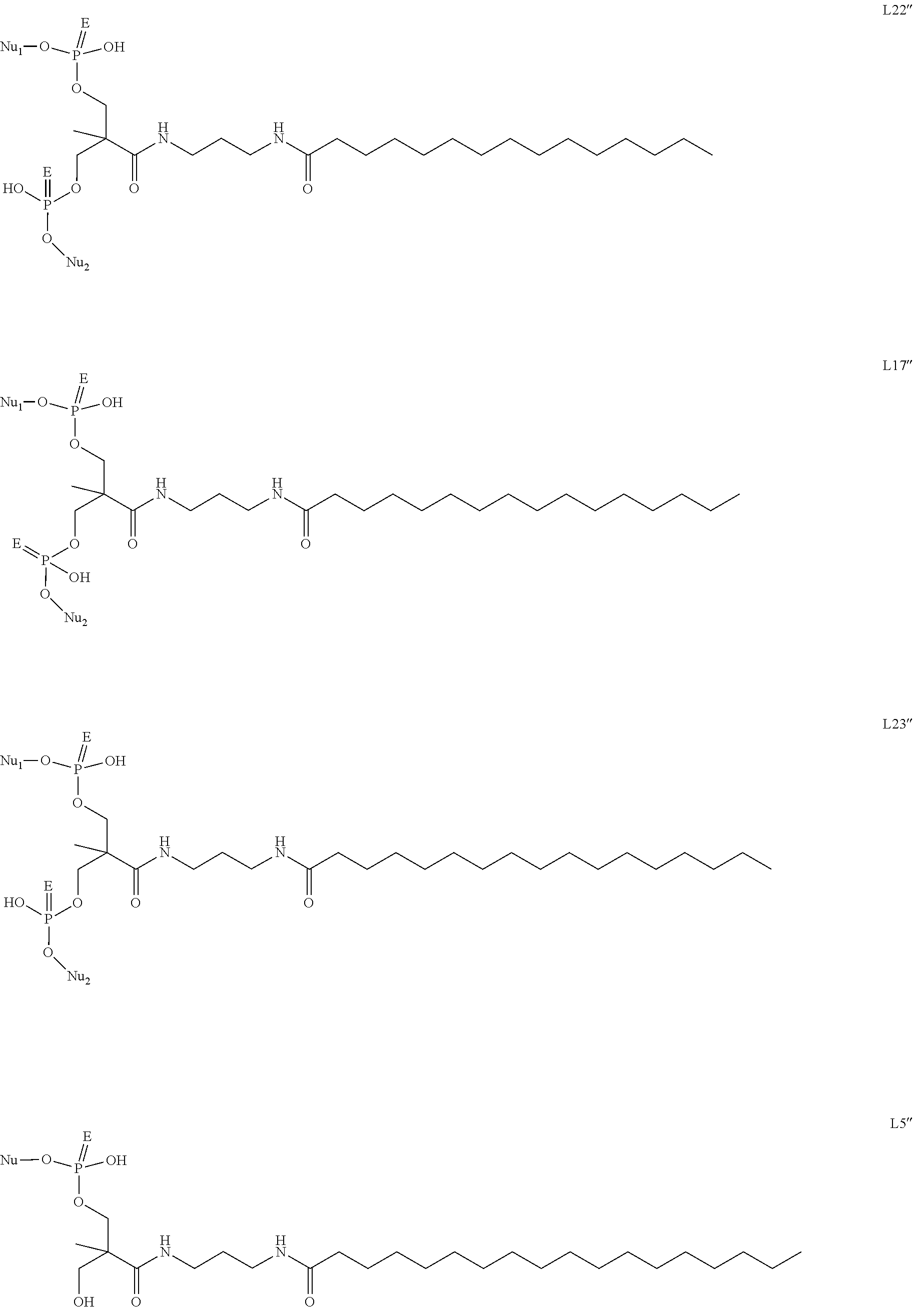

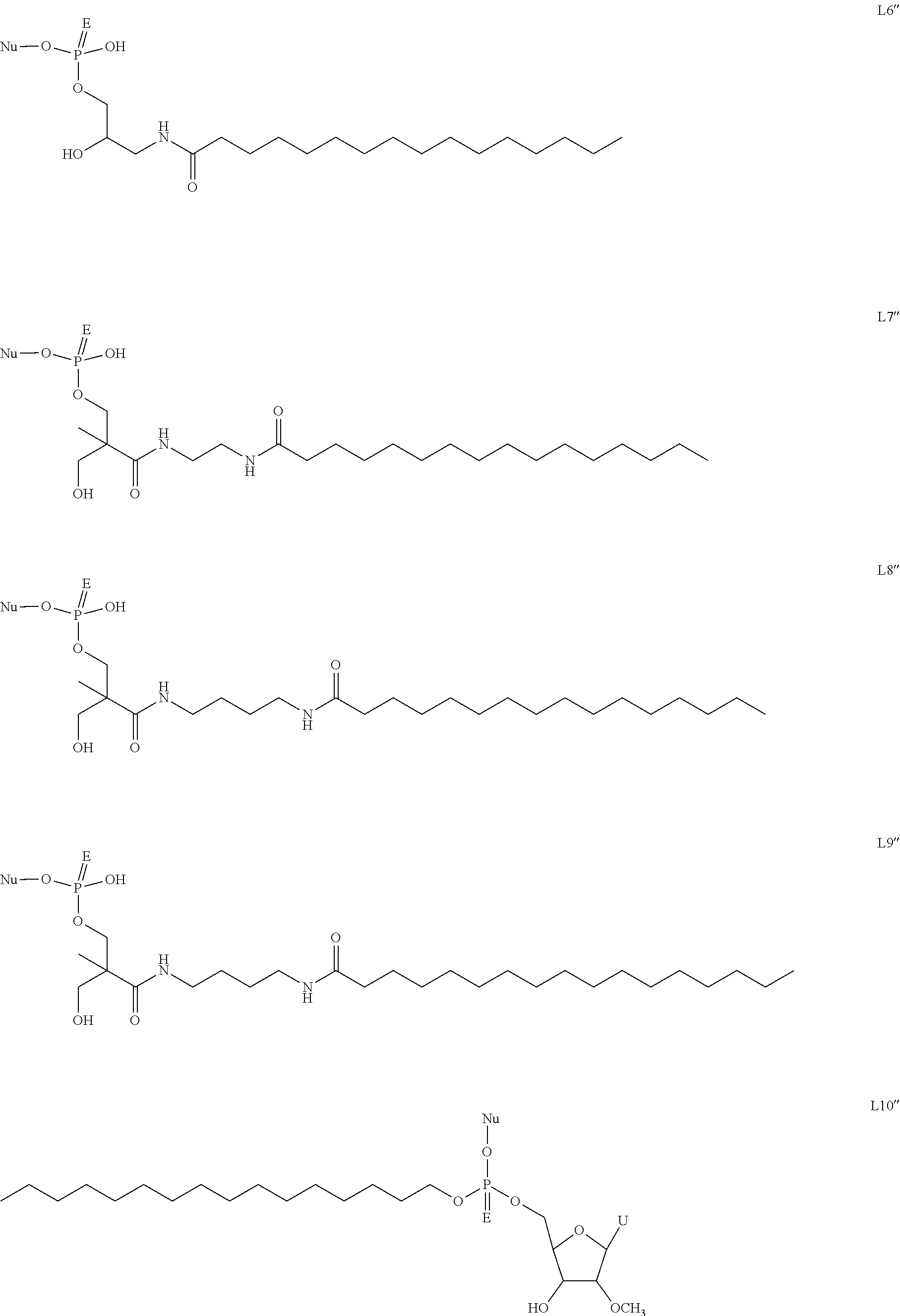

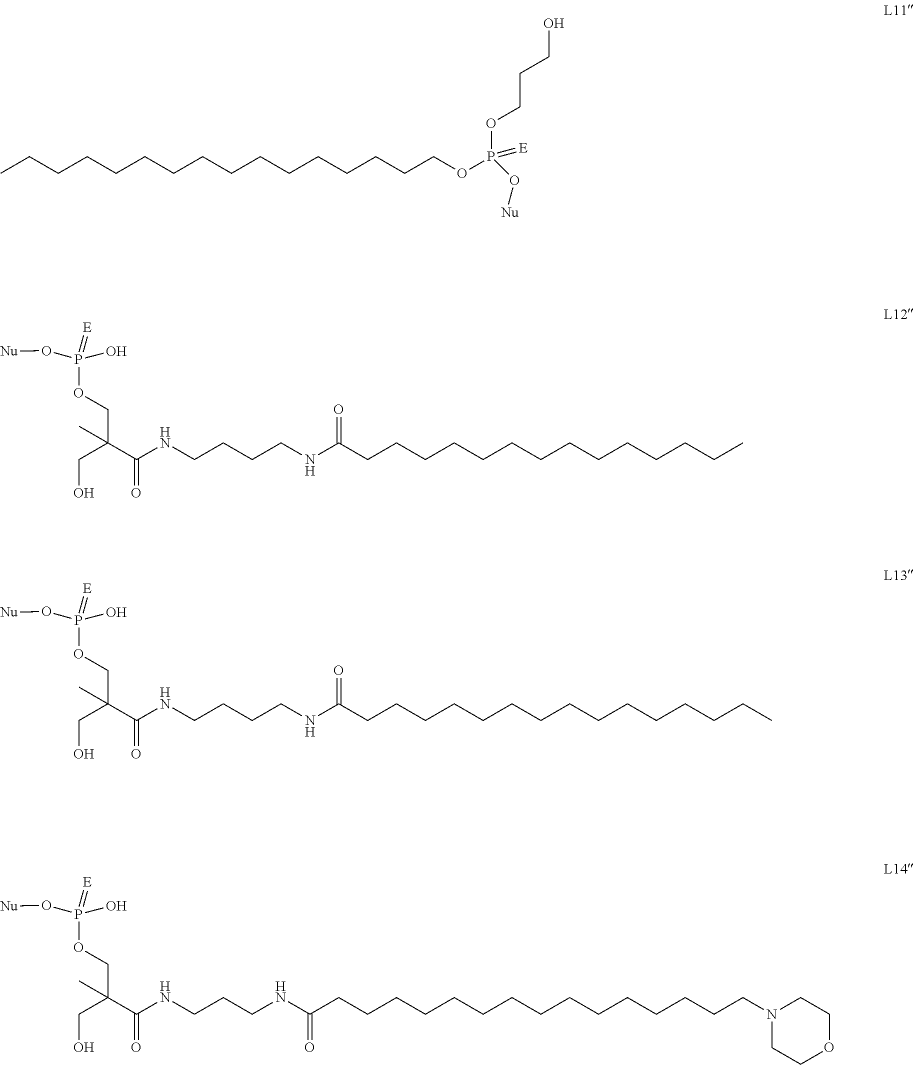

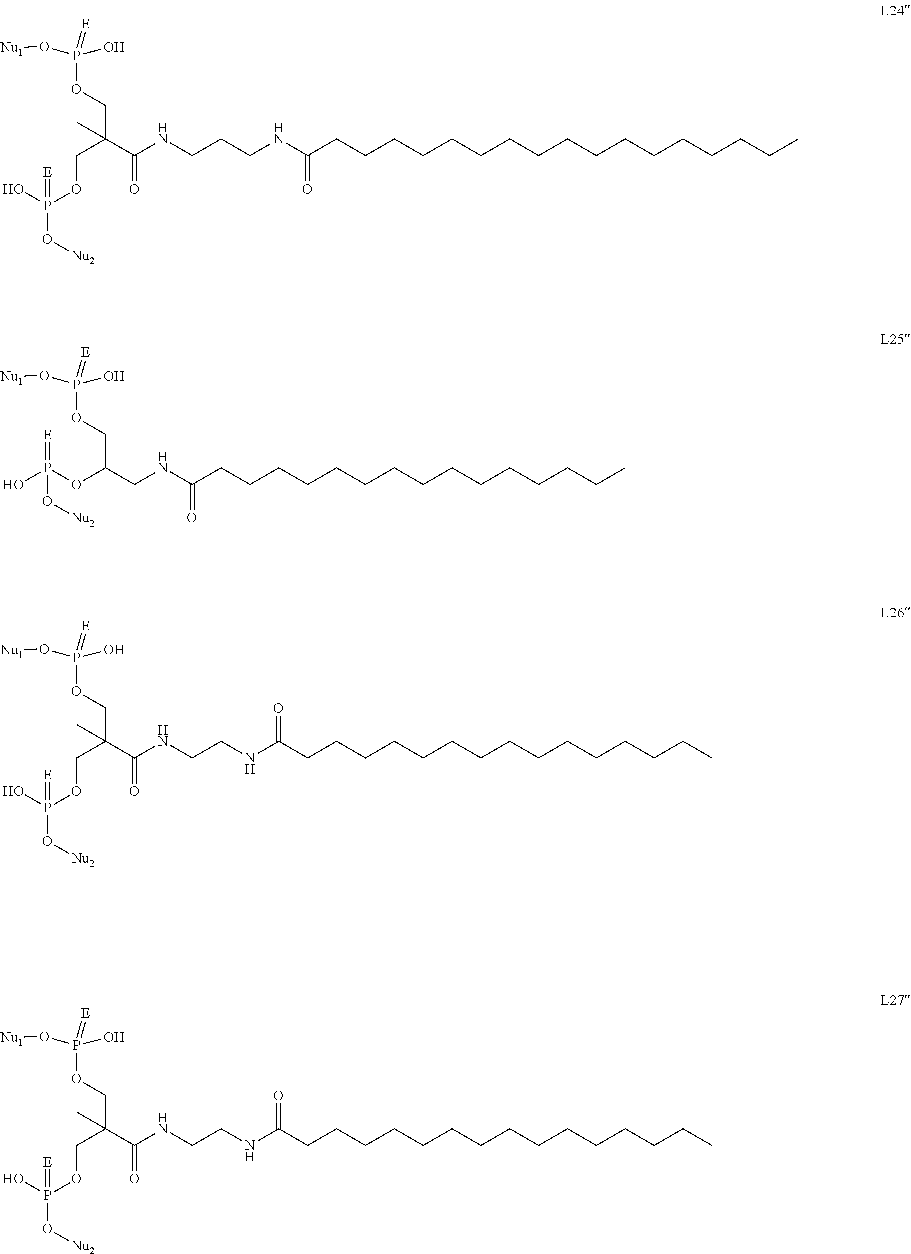

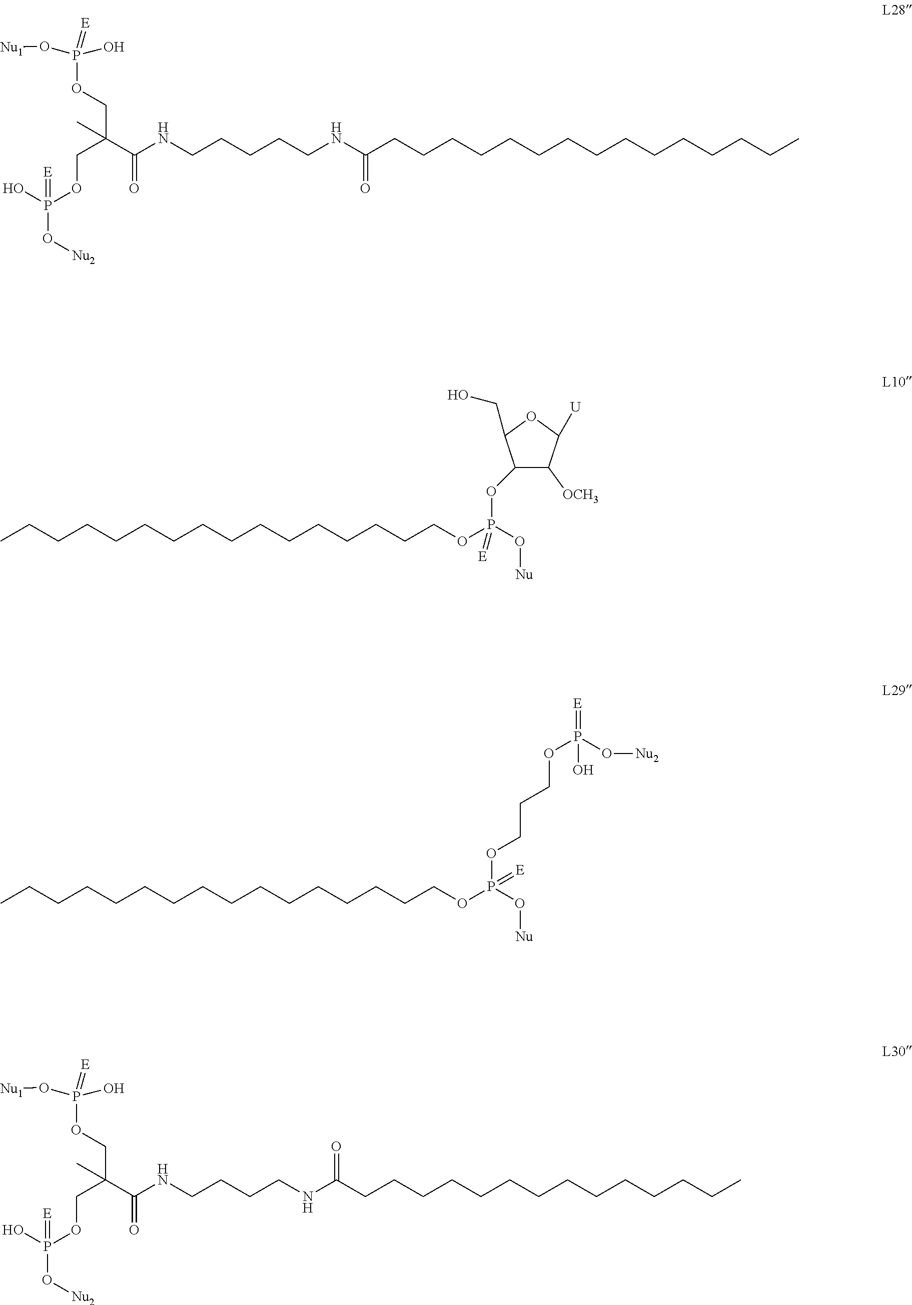

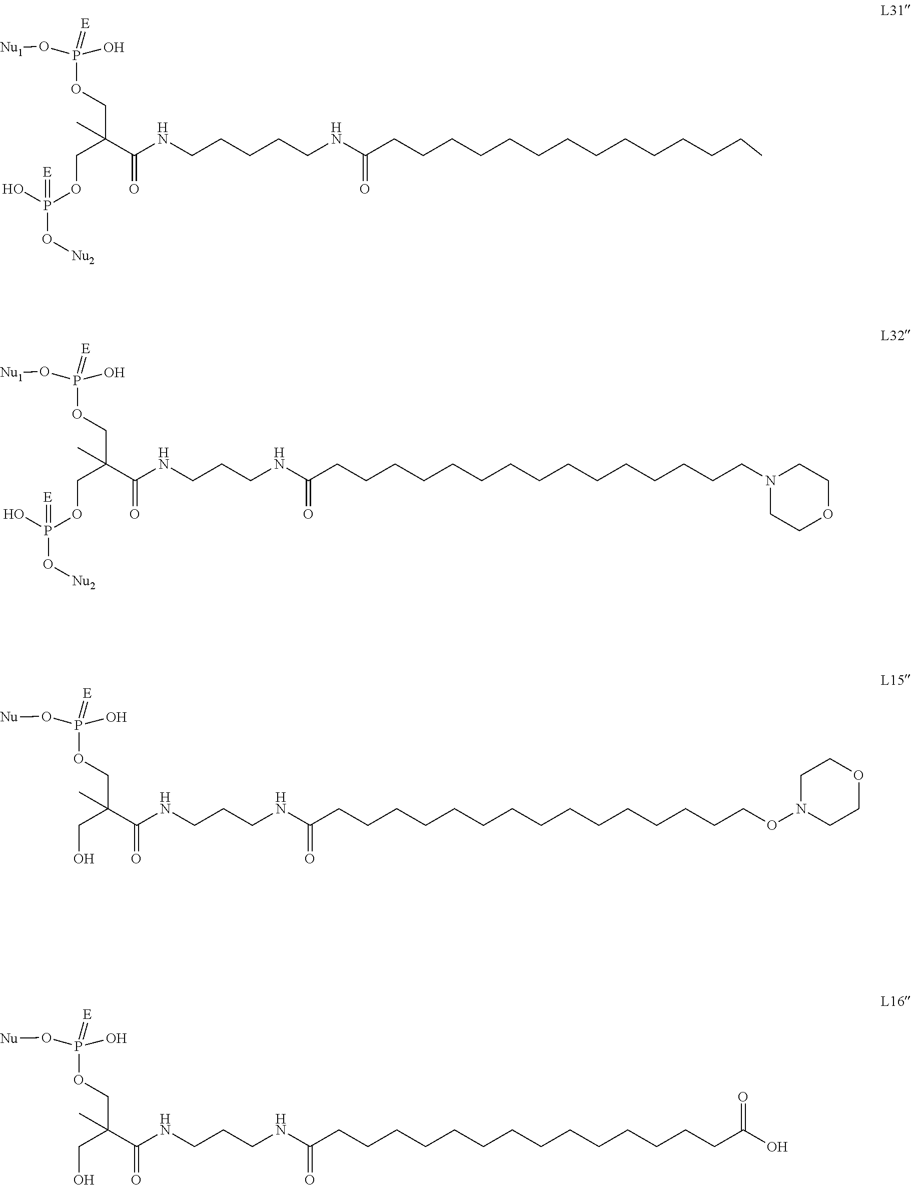

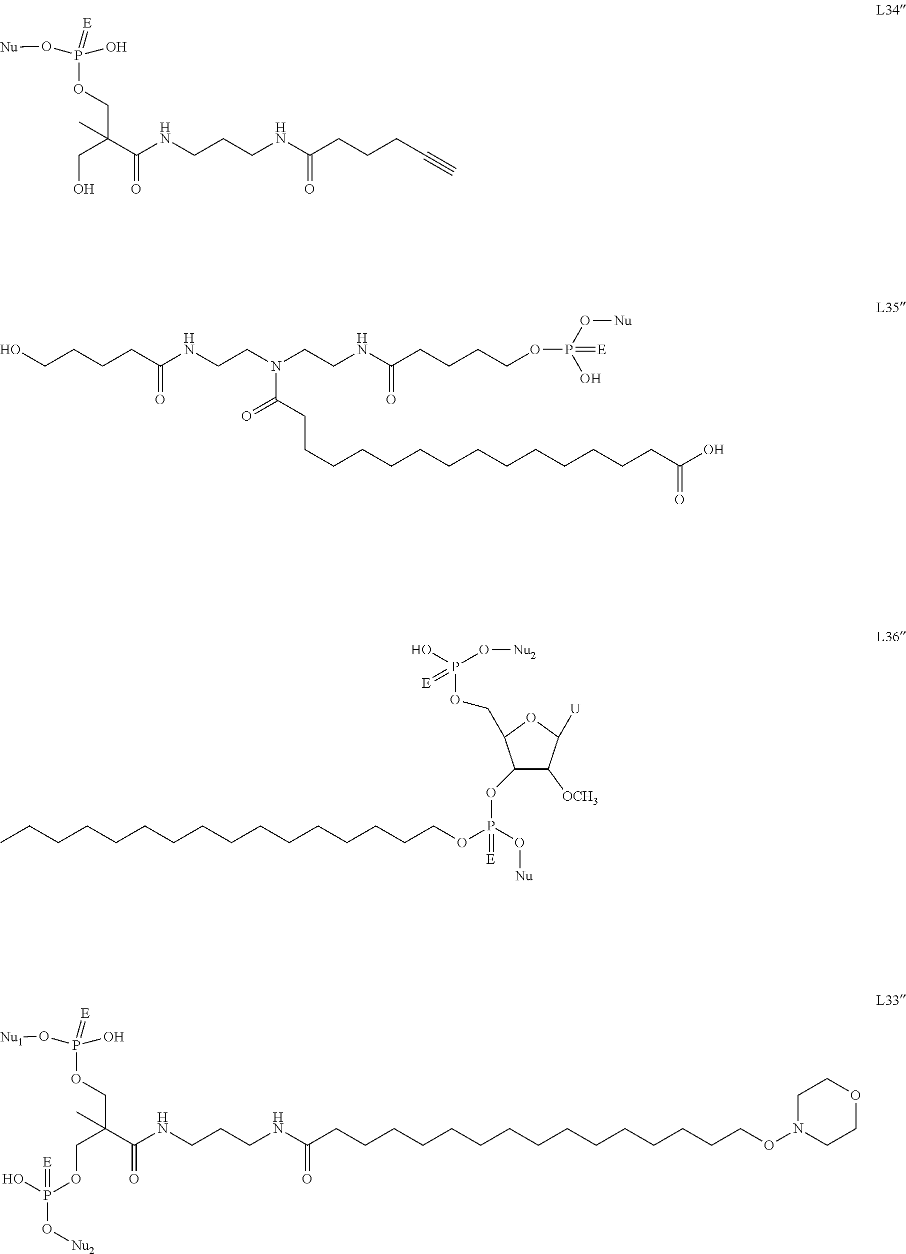

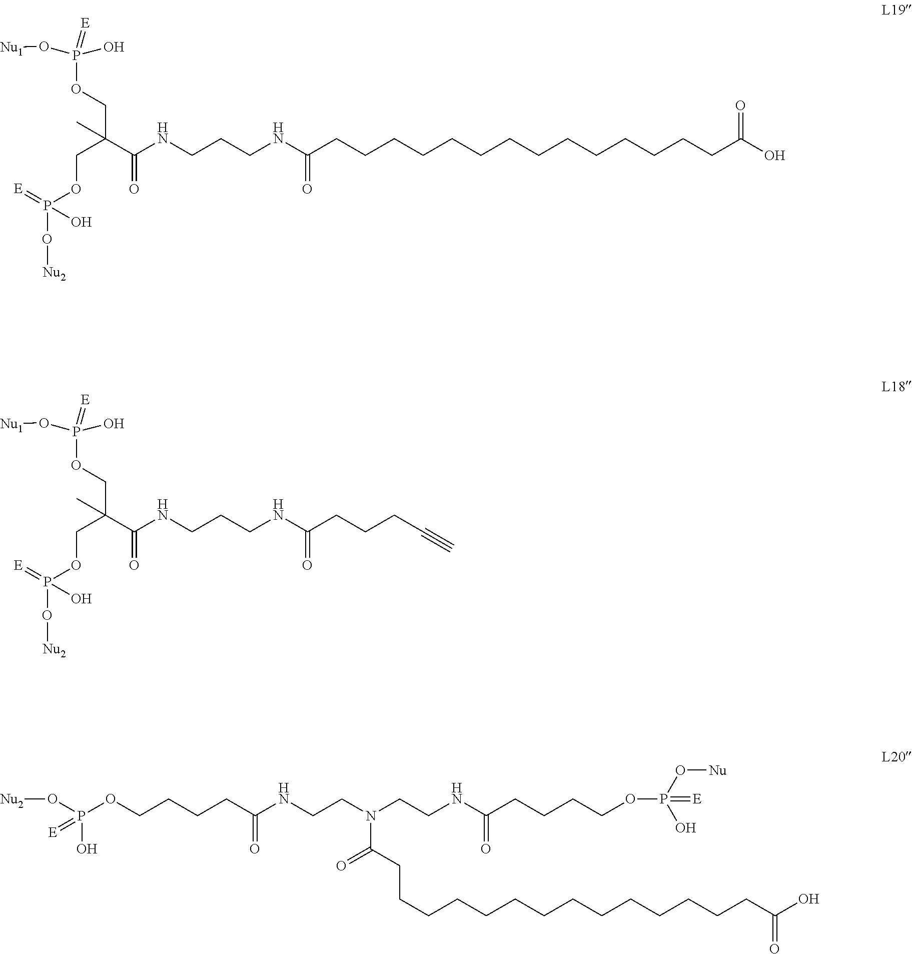

wherein Nu, Nu₁, and Nu₂ independently represent a nucleic acid or a fragment of a nucleic acid; Nu, Nu₁, and Nu₂ may be the same or different; and
wherein E is selected from O or S.

### Cell

The present disclosure also provides an isolated cell containing any of the aforementioned nucleic acids or any of the aforementioned targeted drug delivery systems.

In some embodiments, the cell can be used for purposes such as gene function research, disease model research, or drug screening.

In some embodiments, the cell will not develop into an animal individual. In some specific embodiments, the cell can be a microbial cell or an animal cell, but the animal cell is not an animal embryonic stem cell or a cell at various stages of formation and development thereof (e.g., germ cells, fertilized egg cells, etc.).

### Pharmaceutical Composition

The present disclosure also provides a pharmaceutical composition, which contains any of the aforementioned nucleic acids or any of the aforementioned targeted drug delivery systems, and a pharmaceutically acceptable carrier.

The pharmaceutical composition can be prepared from the nucleic acid and the pharmaceutically acceptable carrier by conventional methods in the art. For example, the pharmaceutical composition can be an injectable solution. The injectable solution can be used for subcutaneous, intramuscular, or intravenous injection.

According to the pharmaceutical composition of the present disclosure, there are no specific requirements for the amounts of the nucleic acid or targeted drug delivery system and the pharmaceutically acceptable carrier. Generally, relative to 1 part by weight of the nucleic acid (or 1 part by weight of the targeted drug delivery system calculated as nucleic acid), the content of the pharmaceutically acceptable carrier can be 1-100,000 parts by weight (e.g., 1 part by weight, 5 parts by weight, 10 parts by weight, 50 parts by weight, 100 parts by weight, 500 parts by weight, 1,000 parts by weight, 5,000 parts by weight, 10,000 parts by weight, 50,000 parts by weight, 100,000 parts by weight, or any value between any two of these numbers).

According to the pharmaceutical composition of the present disclosure, the pharmaceutically acceptable carrier can be various carriers conventionally used in the art, for example, including at least one of pH buffers, protectants, and osmotic pressure regulators. The pH buffer can be a Tris-HCl buffer with a pH of 7.5-8.5 and/or a phosphate buffer with a pH of 5.5-8.5, preferably a phosphate buffer with a pH of 5.5-8.5. The protectant can be at least one of inositol, sorbitol, and sucrose. Based on the total weight of the pharmaceutical composition, the content of the protectant can be 0.01-30% by weight (e.g., 0.01% by weight, 0.05% by weight, 0.1% by weight, 0.5% by weight, 1% by weight, 5% by weight, 10% by weight, 15% by weight, 20% by weight, 25% by weight, 30% by weight, or any value between these numbers). The osmotic pressure regulator can be sodium chloride and/or potassium chloride. The content of the osmotic pressure regulator should be sufficient to make the osmotic pressure of the pharmaceutical composition 200-700 milliosmoles per kilogram. Those skilled in the art can determine the content of the osmotic pressure regulator according to the required osmotic pressure.

According to a preferred embodiment of the present disclosure, the pharmaceutically acceptable carrier is a liposome. The liposome can be any liposome capable of encapsulating nucleic acids, with a diameter of 25-1000 nm, and may include but is not limited to cholesterol and its analogs or derivatives.

The dosage of the pharmaceutical composition of the present disclosure can be a conventional dosage in the art, and the dosage can be determined according to various parameters, especially the age, weight, and gender of the subject. For example, for female mice aged 3-4 months and weighing 25-30 g, the dosage of the pharmaceutical composition, based on the amount of the nucleic acid in the pharmaceutical composition, can be 0.01-100 mg/kg body weight, preferably 1-10 mg/kg body weight.

### Method and Use

The present disclosure also provides a method for inhibiting the expression of MAPT (Tau) in cells, which comprises: contacting the cells with any of the aforementioned nucleic acids, any of the aforementioned targeted drug delivery systems, or the aforementioned pharmaceutical composition to inhibit the expression of MAPT (Tau) in the cells.

In some embodiments, the cells are in a subject, for example, a human subject, such as a subject suffering from a MAPT (Tau)-related disease or a subject in need of preventing the risk of a MAPT (Tau)-related disease.

In some embodiments, the cells are located in vitro. The method is for research purposes or for constructing animal models.

In some embodiments, contacting the cells with the nucleic acid, the targeted drug delivery system, or the pharmaceutical composition inhibits the expression of MAPT (Tau) by at least 50%, 60%, 70%, 80%, 90%, or 95% (for example, compared to the expression level of MAPT (Tau) before the cells first contact the nucleic acid, the targeted drug delivery system, or the pharmaceutical composition; for example, before administering the first dose of the nucleic acid, the targeted drug delivery system, or the pharmaceutical composition to the subject). In certain embodiments, inhibiting the expression of MAPT (Tau) reduces the level of MAPT (Tau) protein in a serum sample from the subject by at least 50%, 60%, 70%, 80%, 90%, or 95%, for example, compared to the expression level of MAPT (Tau) before the cells first contact the nucleic acid, the targeted drug delivery system, or the pharmaceutical composition.

The present disclosure also provides the use of any of the aforementioned nucleic acids, any of the aforementioned targeted drug delivery systems, or the aforementioned pharmaceutical composition in treating and/or preventing MAPT (Tau)-related diseases. That is, a method for treating and/or preventing MAPT (Tau)-related diseases, which comprises: administering any of the aforementioned nucleic acids, any of the aforementioned targeted drug delivery systems, or the aforementioned pharmaceutical composition to a subject.

The present disclosure also provides the use of any of the aforementioned nucleic acids, any of the aforementioned targeted drug delivery systems, or the aforementioned pharmaceutical composition in preparing a medicament for treating and/or preventing MAPT (Tau)-related diseases. That is, a method for preparing a medicament for treating and/or preventing MAPT (Tau)-related diseases, which comprises using any of the aforementioned nucleic acids, any of the aforementioned targeted drug delivery systems, or the aforementioned pharmaceutical composition.

In some embodiments, the disease is: (i) a disease associated with increased or elevated MAPT (Tau); or (ii) a disease that would benefit from reduced MAPT (Tau) expression.

In some embodiments, the disease is selected from one or more of the following: Alzheimer's disease (AD), frontotemporal dementia (FTD), progressive supranuclear palsy (PSP), corticobasal ganglionic degeneration (CBD), Pickle's disease (PiD), agranulocytosis granulomatosa (AGD), subacute sclerosing proptosis total encephalitis (SSPE), Christiansen's syndrome (CS), post-encephalitis Parkinson's syndrome (PEP), Guadeloupe Parkinson's syndrome (GP), globoid glial cell tauopathy (GGT), spinal cerebellar ataxia type 11 (SCA11), chronic traumatic encephalopathy (CTE), aging-associated tau astrocytosis (ARTAG), and primary age-related tauopathy (PART).

In the present disclosure, the subject can be a mammal, including primates (such as humans, non-human primates, e.g., monkeys and chimpanzees), non-primates (such as cattle, pigs, horses, goats, rabbits, sheep, hamsters, guinea pigs, cats, dogs, rats, or mice), or birds. In some embodiments, the subject is preferably a primate, more preferably a human.

In some embodiments, administration can be carried out through various routes, depending on whether local or systemic treatment is needed. The dosage can be referred to as previously mentioned, and will not be repeated here.

In some embodiments, administration can be local (e.g., transdermal patch), pulmonary, such as inhalation or insufflation via powder or spray, including via nebulizer; intratracheal, nasal, topical, transdermal, oral, or parenteral. Parenteral administration includes intravenous, intra-arterial, subcutaneous, intraperitoneal, or intramuscular injection or infusion; subcutaneous, such as via an implantation device; or intracranial, such as intraparenchymal, intrathecal, or intraventricular administration.

In some embodiments, the nucleic acid, the targeted drug delivery system, or the pharmaceutical composition is administered to the subject via subcutaneous administration, intravenous administration, intramuscular administration, and/or intracranial administration.

### Examples

The embodiments of the present disclosure will be described in detail below with reference to examples. It should be understood that these examples are only used to illustrate the present disclosure and are not intended to limit the scope of the present disclosure. For the experimental methods without specific conditions indicated in the following examples, priority is given to the guidelines provided in the present disclosure, and they can also be carried out in accordance with experimental manuals or conventional conditions in the art, or with reference to other known experimental methods in the art, or in accordance with the conditions suggested by the manufacturers.

In the specific examples described below, regarding the measurement parameters of raw material components, unless otherwise specified, there may be slight deviations within the range of weighing accuracy. For temperature and time parameters, acceptable deviations caused by instrument testing accuracy or operation accuracy are allowed.

### I. Methods

SH-SY5Y cell culture and nucleofection of siRNA: The neuroblastoma SH-SY5Y cell line (CellBank, #SCSP-0514) was cultured in a medium containing 1x DMEM+10% fetal bovine serum (FBS)+1% penicillin-streptomycin. The SF Cell Line 4D-Nucleofector X Kit and Nucleocuvette Strip (Lonza, #V4XC-2032) were used. On the day of nucleofection, 30,000 SH-SY5Y cells were resuspended in 20 μL Nucleofector Solution (+ Supplement). siRNA (500 nM or 20 nM) was added or not added according to experimental groups, and electroporation was performed under the “CA-137” program. After transfection, the entire 20 μL content in the Nucleocuvette was mixed with 80 μL pre-warmed 1x DMEM+10% FBS and transferred to a 96-well plate. At 24 hours post-transfection, qRT-PCR was performed using the Cells-to-CT Kit (ThermoFisher). The Taqman primer set HS00902194_m1 (FAM-MGB) was used to detect the MAPT gene, and HS01060665_g1 (VIC-MGB) was used to detect ACTB as an internal control. Dose response experiments with different concentrations of siRNA were also conducted according to the above method.

Hep3B cell culture and transfection of siRNA: The hepatocarcinoma Hep3B cell line (Procell, #CL-0102) was plated in 96-well plates at a density of 13,000 cells per well (medium as described above). Reverse transfection was performed using 0.3 μL Lipofectamine RNAiMax (according to the manufacturer's protocol) at a range of siRNA concentrations (up to 10 nM) for 48 hours. Finally, the cells were harvested and quantified using the Cells-to-CT Kit. The Taqman primer set HS02800695_m1 (VIC-MGB) was used to detect the internal control gene HPRT1 (ThermoFisher).

iNeuron cell culture and transfection of siRNA: iNeurons were generated in-house from an induced pluripotent stem cell line (iPSC, Sigma-Aldrich, #UOXFi006-A) using the plasmid CLYBL-TO-hNGN2-BSD-mApple (Addgene, #124229) and the CRISPR-Cas9 protocol included therein. The edited iPSCs were plated on Matrigel-coated 6-well plates in mTeSR plus medium (STEMCELL, #100-0276) supplemented with 10 M Y-27632 ROCK inhibitor (STEMCELL, #72302) on day zero. For the next two days, the medium was changed daily to NM (DMEM F12+2 mg/ml heparin+1x B-27 (Gibco)+1x N2 (Gibco)+1x non-essential amino acids+1x Glutamax+2 g/ml doxycycline). On day three, cells were re-plated in NM medium at 1.2×10⁶ per well in the presence or absence of siRNA and Lipofectamine RNAiMax. Mature iNeurons (with nearly 100% NeuN expression) were harvested on differentiation day five for qRT-PCR.

### II. Results

1. The siRNAs in Table 2 were diluted with ddH₂O to a final concentration of 10 nM, transfected into SH-SY5Y cells using RNAiMax, and their in vitro activity was detected 48 hours post-transfection (biological replicates n=3). The results are shown in Table 4 below:

TABLE 4

| | % Avg remaining | | | % Avg remaining | |
|---|---|---|---|---|---|
| siRNA | MAPT mRNA | Std | siRNA | MAPT mRNA | Std |
| SN-5161 | 39.3 | 5.0 | SN-5104 | 58.1 | 1.2 |
| SN-5162 | 43.8 | 3.5 | SN-5105 | 54.9 | 9.5 |
| SN-5173 | 37.8 | 1.5 | SN-5106 | 54.7 | 7.1 |
| SN-5174 | 49.2 | 6.4 | SN-5107 | 53.9 | 5.9 |
| SN-5175 | 58.8 | 8.7 | SN-5108 | 49.1 | 1.6 |
| SN-5176 | 50.8 | 4.4 | SN-5109 | 57.3 | 4.2 |
| SN-5177 | 41.1 | 1.1 | SN-5100 | 57.4 | 3.7 |
| SN-5178 | 52.1 | 1.7 | SN-5101 | 56.4 | 4.7 |
| SN-5179 | 54.8 | 3.4 | SN-5102 | 54.7 | 0.8 |
| SN-5170 | 51.2 | 4.9 | SN-5113 | 57.5 | 4.3 |
| SN-5171 | 61.8 | 2.0 | SN-5114 | 57.9 | 3.9 |
| SN-5172 | 59.1 | 6.0 | SN-5115 | 59.7 | 2.7 |
| SN-5183 | 45.4 | 4.0 | SN-5116 | 54.9 | 7.5 |
| SN-5185 | 45.9 | 0.6 | SN-5117 | 61.6 | 2.8 |
| SN-5186 | 41.7 | 3.7 | SN-5118 | 68.4 | 2.4 |
| SN-5187 | 42.8 | 4.9 | SN-5119 | 59.5 | 4.6 |
| SN-5188 | 41.4 | 1.1 | SN-5110 | 66.9 | 2.0 |
| SN-5180 | 35.1 | 4.2 | SN-5111 | 65.2 | 8.8 |
| SN-5181 | 37.9 | 1.1 | SN-5112 | 66.3 | 2.3 |
| SN-5182 | 30.5 | 3.1 | SN-5103 | 62.2 | 6.1 |
| SN-5193 | 40.5 | 4.2 | SN-5104 | 53.7 | 5.8 |
| SN-5194 | 32.2 | 6.1 | SN-5105 | 57.5 | 6.5 |
| SN-5195 | 46.7 | 7.6 | SN-5106 | 56.5 | 6.6 |
| SN-5196 | 48.3 | 4.1 | SN-5107 | 57.4 | 2.4 |
| SN-5197 | 51.3 | 5.5 | SN-5108 | 65.1 | 2.8 |
| SN-5198 | 48.5 | 4.4 | SN-5109 | 61.0 | 2.6 |
| SN-5199 | 48.6 | 1.8 | SN-5120 | 63.5 | 8.1 |
| SN-5190 | 58.9 | 9.3 | SN-5121 | 69.9 | 3.8 |
| SN-5191 | 72.3 | 2.2 | SN-5122 | 76.8 | 1.3 |
| SN-5192 | 66.5 | 9.5 | SN-5233 | 76.1 | 12.5 |
| SN-5103 | 63.4 | 4.1 | | | |

2. The siRNAs selected from Table 4 were further evaluated in SH-SY5Y cells by nucleofection, and their in vitro activity against endogenous MAPT mRNA was detected 24 hours post-nucleofection (biological replicates n=2). The results are summarized in Table 5.

TABLE 5

| | |
|---|---|
| 20 nM | 500 nM |

| | % Avg remaining | | % Avg remaining | |
|---|---|---|---|---|
| siRNA | MAPT mRNA | Std | MAPT mRNA | Std |
| SN-5180 | 59.3 | 7.2 | 21.8 | 2.9 |
| SN-5182 | 32.9 | 5.4 | 14.1 | 3.4 |
| SN-5194 | 52.8 | 8.6 | 29.8 | 1.9 |
| SN-5196 | 30.6 | 2.1 | 37.8 | 1.9 |
| SN-5107 | 48.1 | 5.0 | 30.9 | 5.0 |
| SN-5114 | 37.5 | 5.1 | 32.1 | 3.5 |
| SN-5124 | 51.6 | 1.0 | 35.6 | 1.7 |

3. The siRNAs selected from Table 4 were further evaluated in iNeuron cells by nucleofection. The in vitro activity of the siRNA compounds in transfected iNeurons on differentiation day five was detected (biological replicates n=3), and the results are shown in Table 6; the dose response results in transfected iNeurons on differentiation day five were detected (biological replicates n=3), and the results are shown in Table 7.

TABLE 6

| | 500 nM | | 100 nM | |
|---|---|---|---|---|
| | % AVG remaining | | % AVG remaining | |
| siRNA | MAPT mRNA | SD | MAPT mRNA | SD |
| SN-5182 | 13.4 | 0.6 | 30.1 | 7.7 |
| SN-5180 | 52.4 | 9.1 | 61.4 | 3.5 |
| SN-5194 | 48.5 | 1.2 | 71.5 | 5.3 |
| SN-5107 | 49.1 | 1.9 | 48.9 | 10.1 |
| SN-5114 | 58.4 | 4.3 | 62.1 | 6.2 |
| SN-5124 | 53.1 | 2.2 | 42.5 | 5.3 |
| SN-5196 | 40.2 | 2.3 | 34.4 | 3.3 |

TABLE 7

| | 10 nM | | 5 nm | | 2.5 nM | | 1 nM | | 0.5 nM | | 0.25 nM | | 0.1 nM | |
|---|---|---|---|---|---|---|---|---|---|---|---|---|---|---|
| | % AVG | | % AVG | | % AVG | | % AVG | | % AVG | | % AVG | | % AVG | |
| | remaining | | remaining | | remaining | | remaining | | remaining | | remaining | | remaining | |
| | MAPT | | MAPT | | MAPT | | MAPT | | MAPT | | MAPT | | MAPT | |
| siRNA | mRNA | SD | mRNA | SD | mRNA | SD | mRNA | SD | mRNA | SD | mRNA | SD | MRNA | SD |
| SN-5182 | 38.5 | 3.8 | 38.0 | 3.3 | 40.2 | 8.3 | 37.7 | 7.9 | 37.7 | 1.9 | 46.4 | 1.6 | 70.9 | 7.3 |
| SN-5180 | 58.3 | 19.2 | 56.6 | 12.8 | 42.6 | 0.9 | 46.0 | 2.4 | 53.6 | 4.9 | 56.1 | 9.3 | 85.4 | 10.8 |
| SN-5184 | 122.3 | 17.2 | 116.4 | 13.8 | 90.1 | 7.4 | 68.9 | 7.3 | 59.3 | 4.9 | 84.7 | 6.2 | 87.7 | 3.4 |
| SN-5107 | 57.7 | 22.1 | 48.8 | 14.7 | 46.9 | 2.3 | 49.1 | 1.7 | 57.8 | 5.4 | 55.3 | 2.6 | 78.6 | 5.2 |
| SN-5114 | 78.1 | 11.0 | 69.9 | 7.3 | 64.0 | 8.0 | 59.6 | 2.9 | 65.2 | 8.6 | 58.6 | 3.9 | 76.0 | 10.1 |
| SN-5124 | 52.6 | 6.1 | 54.2 | 10.7 | 51.1 | 6.5 | ND | ND | 63.8 | 3.8 | ND | ND | 65.7 | 6.0 |
| SN-5196 | 63.8 | 6.8 | 62.3 | 10.9 | 53.7 | 4.1 | 58.0 | 1.1 | 64.6 | 3.8 | 68.9 | 7.1 | 86.1 | 1.7 |

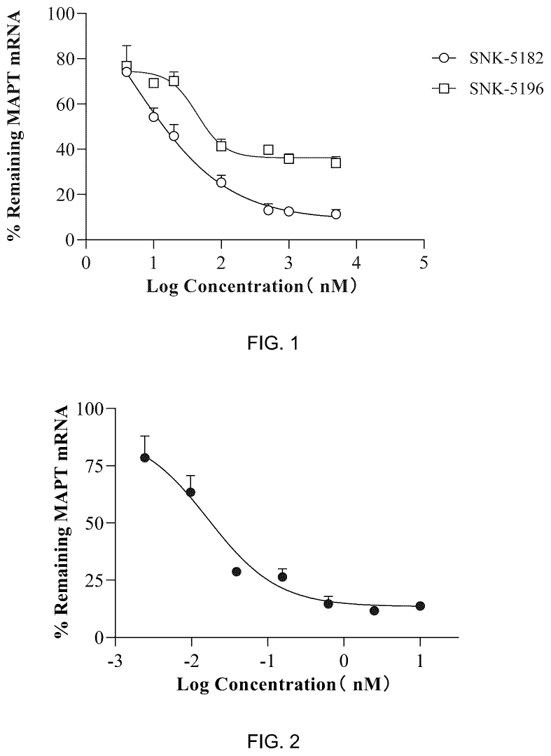

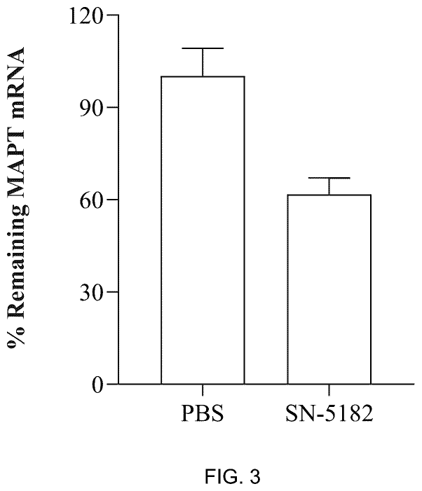

In the tables, ND indicates no data.
4. SN-5182 and SN-5196 were further evaluated in SH-SY5Y cells by nucleofection, and the in vitro dose response curves of SN-5182 and SN-5196 against endogenous MAPT mRNA in SH-SY5Y cells 24 hours post-nucleofection were detected (biological replicates n=2). The results are shown in FIG. 1.
5. SN-5182 was further evaluated in Hep3B cells by nucleofection, and the in vitro dose response curve of SN-5182 against endogenous MAPT mRNA in Hep3B cells 48 hours post-transfection was detected (biological replicates n=3). The results are shown in FIG. 2.
6. The different modified sequences related to SN-5182 shown in Table 3 were further evaluated in Hep3B cells. Their IC₅₀ values of the in vitro dose response curves against endogenous MAPT mRNA in Hep3B cells 48 hours post-transfection were detected (biological replicates n=2). The results are shown in Table 8.

TABLE 8

| siRNA | IC50, nM |
|---|---|
| SN-6725 | 0.057 |
| SN-6726 | 0.567 |
| SN-5182 | 0.011 |

It can be seen from the above that the siRNAs of the present disclosure (especially including SN-5182) can significantly reduce the expression level of MAPT in cells, showing great potential in the preparation of RNAi drugs for Tau-related diseases.

The above-mentioned embodiments only express several implementation modes of the present disclosure, and their descriptions are relatively specific and detailed, but they should not be understood as limiting the protection scope of the present disclosure. It should be pointed out that for those of ordinary skill in the art, without departing from the concept of the present disclosure, several modifications and improvements can also be made, and these all belong to the protection scope of the present disclosure.

1. A nucleic acid comprising a sense strand and an antisense strand, wherein the sense strand comprises a sequence having more than 80% sequence identity to any of sequences shown in SEQ ID NO: 1-61, and the antisense strand comprises a sequence having more than 80% sequence identity to any of sequences shown in SEQ ID NO: 62-122.

2. The nucleic acid according to claim 1, wherein the antisense strand comprises at least 15 contiguous nucleotides and the at least 15 contiguous nucleotides of the antisense strand differ by no more than 3 nucleotides from at least 15 contiguous nucleotides of the nucleotide sequences of any of the sequences shown in SEQ ID NO: 62-122, and wherein the sense strand comprises a nucleotide sequence at least partially complementary to the antisense strand; optionally, the sense strand comprises at least 15 contiguous nucleotides and the at least 15 contiguous nucleotides of the sense strand differ by no more than 3 nucleotides from at least 15 contiguous nucleotides of the nucleotide sequences of any of the sequences shown in SEQ ID NO: 1-61.

3. The nucleic acid according to claim 1, wherein the antisense strand comprises a nucleotide sequence differing by 0, 1 or 2 nucleotides from any of sequences shown in SEQ ID NO: 79, 81, 83, 85, 96, 103, 113, and wherein the sense strand comprises a nucleotide sequence at least partially complementary to the antisense strand; optionally, the sense strand comprises a nucleotide sequence differing by 0, 1, or 2 nucleotides from any of sequences shown in SEQ ID NO. 18, 20, 22, 24, 35, 42, 52.

4. The nucleic acid according to claim 1, wherein the sense strand comprises a nucleotide sequence differing by 0, 1, or 2 nucleotides from the sequence shown in SEQ ID NO: 18, and the antisense strand comprises a nucleotide sequence differing by 0, 1, or 2 nucleotides from the sequence shown in SEQ ID NO: 79;
the sense strand comprises a nucleotide sequence differing by 0, 1 or 2 nucleotides from the sequence shown in SEQ ID NO: 20, and the antisense strand comprises a nucleotide sequence differing by 0, 1 or 2 nucleotides from the sequence shown in SEQ ID NO: 81;
the sense strand comprises a nucleotide sequence differing by 0, 1 or 2 nucleotides from the sequence shown in SEQ ID NO: 22, and the antisense strand comprises a nucleotide sequence differing by 0, 1 or 2 nucleotides from the sequence shown in SEQ ID NO: 83;
the sense strand comprises a nucleotide sequence differing by 0, 1 or 2 nucleotides from the sequence shown in SEQ ID NO: 24, and the antisense strand comprises a nucleotide sequence differing by 0, 1 or 2 nucleotides from the sequence shown in SEQ ID NO: 85;
the sense strand comprises a nucleotide sequence differing by 0, 1 or 2 nucleotides from the sequence shown in SEQ ID NO: 35, and the antisense strand comprises a nucleotide sequence differing by 0, 1 or 2 nucleotides from the sequence shown in SEQ ID NO: 96;
the sense strand comprises a nucleotide sequence differing by 0, 1 or 2 nucleotides from the sequence shown in SEQ ID NO: 42, and the antisense strand comprises a nucleotide sequence differing by 0, 1 or 2 nucleotides from the sequence shown in SEQ ID NO: 103; or,
the sense strand comprises a nucleotide sequence differing by 0, 1 or 2 nucleotides from the sequence shown in SEQ ID NO: 52, and the antisense strand comprises a nucleotide sequence differing by 0, 1 or 2 nucleotides from the sequence shown in SEQ ID NO: 113.

5. The nucleic acid according to claim 1, wherein at least one nucleotide of the nucleic acid is a modified nucleotide and/or comprises a modified internucleotide linkage.

6. The nucleic acid according to claim 5, wherein the modified nucleotide is selected from one or more of 2′-O-methylnucleotides, 2′-fluoronucleotides, 2′-deoxyribonucleotides, 2′,3′-open cyclic nucleotide analogs, locked nucleotides, 2′-F-arabinose nucleotide, 2′-methoxyethyl nucleotide, dealkylated nucleotide, ribitol, reverse nucleotide, reverse 2′-O-methyl nucleotide, reverse 2′-deoxyribonucleotides, 2′-amino-modified nucleotides, 2′-alkyl-modified nucleotides, morpholino, peptide nucleic acid (PNA), glycerol nucleic acid (GNA), triazacyclic DNA (tcDNA), nucleotides comprising unnatural bases, nucleotides containing vinyl phosphonates, nucleotides containing cyclopropyl phosphonates, and 3′-O-methyl nucleotides; and
the modified internucleotide linkage is selected from one or more of phosphorothioate internucleotide linkages and methylphosphonate internucleotide linkages.

7. The nucleic acid according to claim 5, the modified nucleotide is selected from one or more of 2′-O-methylnucleotides and 2′-fluoronucleotides; and the modified internucleotide linkage is selected from one or more of phosphorothioate monoester internucleotide linkages and phosphorothioate diester internucleotide linkages.

8. The nucleic acid according to claim 5, wherein the antisense strand comprises a 2′-fluoronucleotide at positions 2, 4, 6, and 16, and optionally at position 14, of any one of the nucleotide sequences shown in SEQ ID NO: 62-122.

9. The nucleic acid according to claim 5, wherein the sense strand comprises a 2′-fluoronucleotide at positions 7, 9, 10, and 11, and optionally at position 18, of any one of the nucleotide sequences shown in SEQ ID NOS. 1-61.

10. The nucleic acid according to claim 5, wherein the antisense strand comprises a phosphorothioate internucleotide linkage between the last 2-4 nucleotides at a 5′ end and/or a 3′ end of the antisense strand, and the sense strand comprises a phosphorothioate internucleotide linkage between the last 2-4 nucleotides at a 5′ end and/or a 3′ end of the sense strand.

11. The nucleic acid according to claim 1, wherein the antisense strand comprises a nucleotide sequence differing by 0, 1 or 2 nucleotides from an antisense strand shown in either Table 2 or Table 3;
wherein the sense strand comprises a nucleotide sequence differing by 0, 1 or 2 nucleotides from a sense strand shown in either Table 2 or Table 3; or
wherein the nucleic acid comprises a double strand shown in either Table 2 or Table 3.

12. The nucleic acid according to claim 11, wherein the nucleic acid comprises a double strand selected from any one of SN-5180, SN-5182, SN-5194, SN-5196, SN-5107, SN-5114, SN-5124, SN-6725, SN-6726.

13. A targeted drug delivery system comprising:
a targeting moiety;
a linking moiety; and
a nucleic acid according to claim 1,
wherein the nucleic acid is linked to the targeting moiety via the linking moiety.

14. The targeted drug delivery system of claim 13, wherein the linking moiety is linked to a 5′ end or a 3′ end of the sense strand of the nucleic acid or a 5′ end or a 3′ end of the antisense strand of the nucleic acid.

15. The targeted drug delivery system according to claim 13, wherein the targeted drug delivery system comprises a ligand and the nucleic acid linked to the ligand, the ligand comprising a structure shown in formula (XI) or formula (XII): 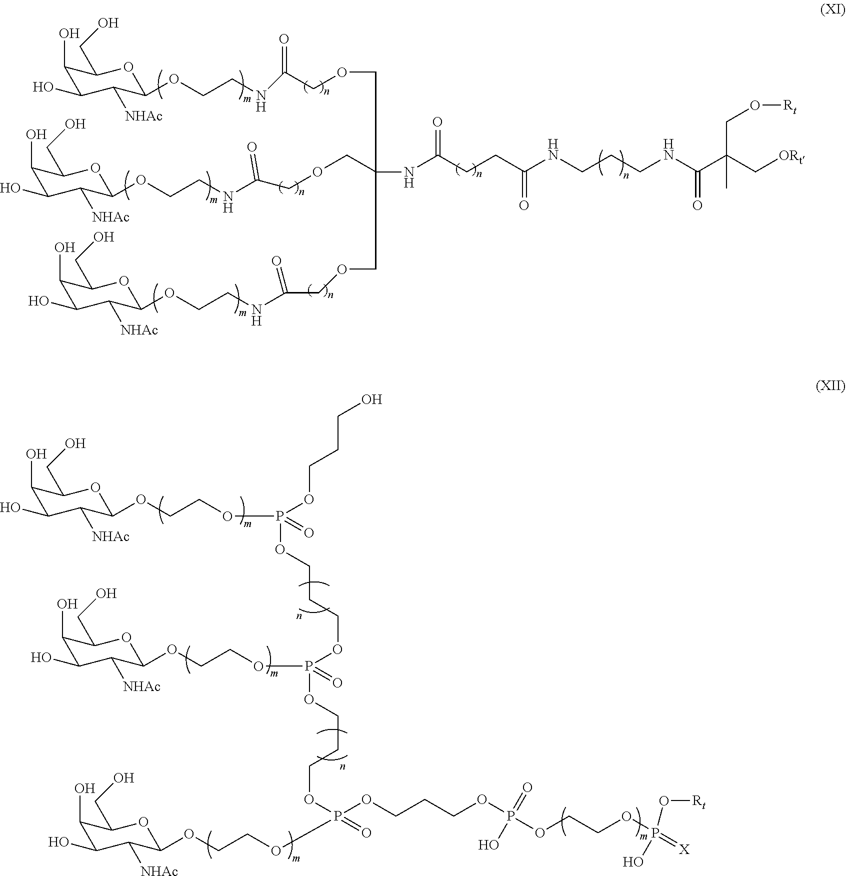
wherein m and n in formula (XI) or formula (XII) are each independently an integer from 0 to 6; and Rt and Rt′ are each independently any one of the nucleic acid.

16. The targeted drug delivery system according to claim 13, wherein the targeted drug delivery system has a structure shown in formula (601), (602), (603), (604), (605), (606) or (607): 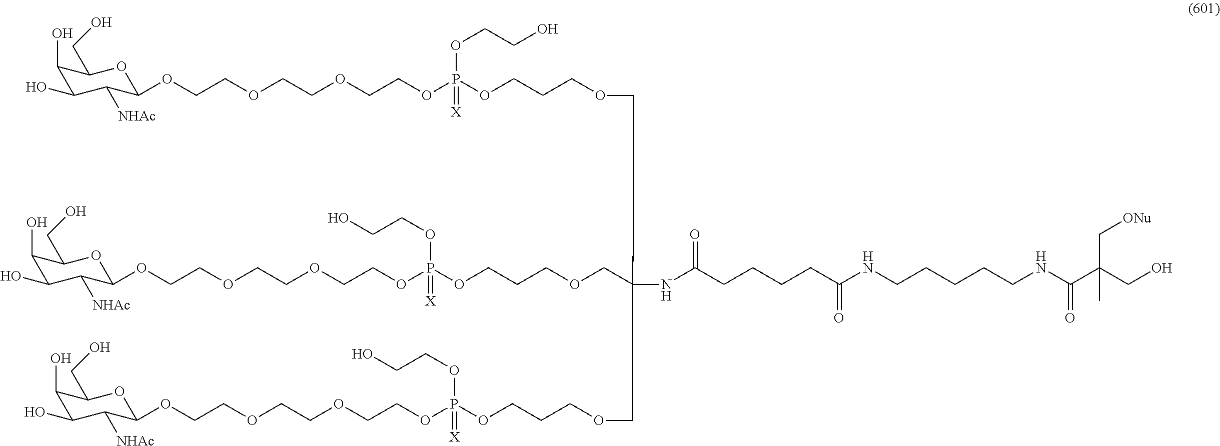 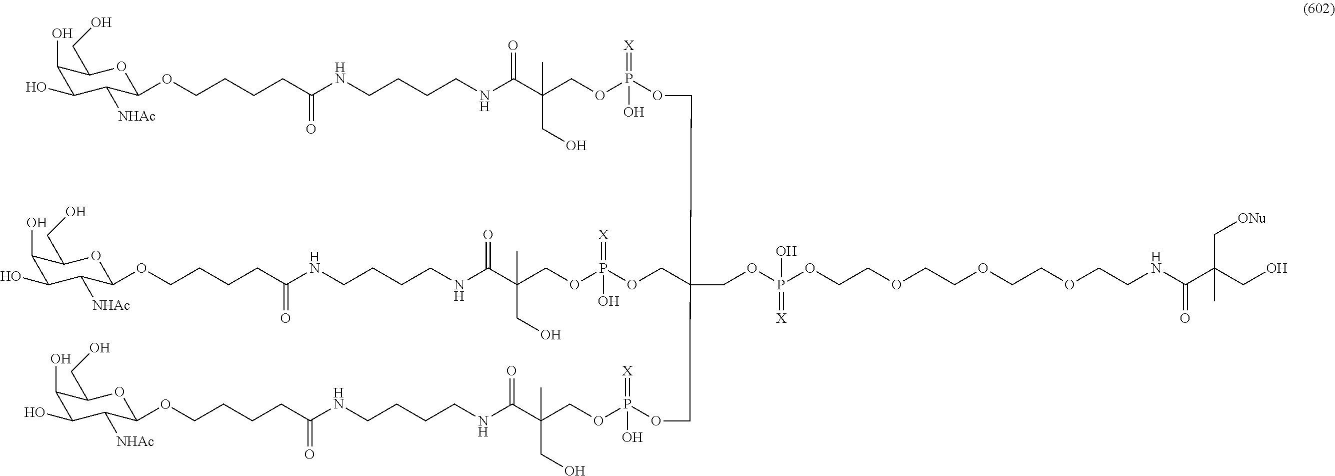 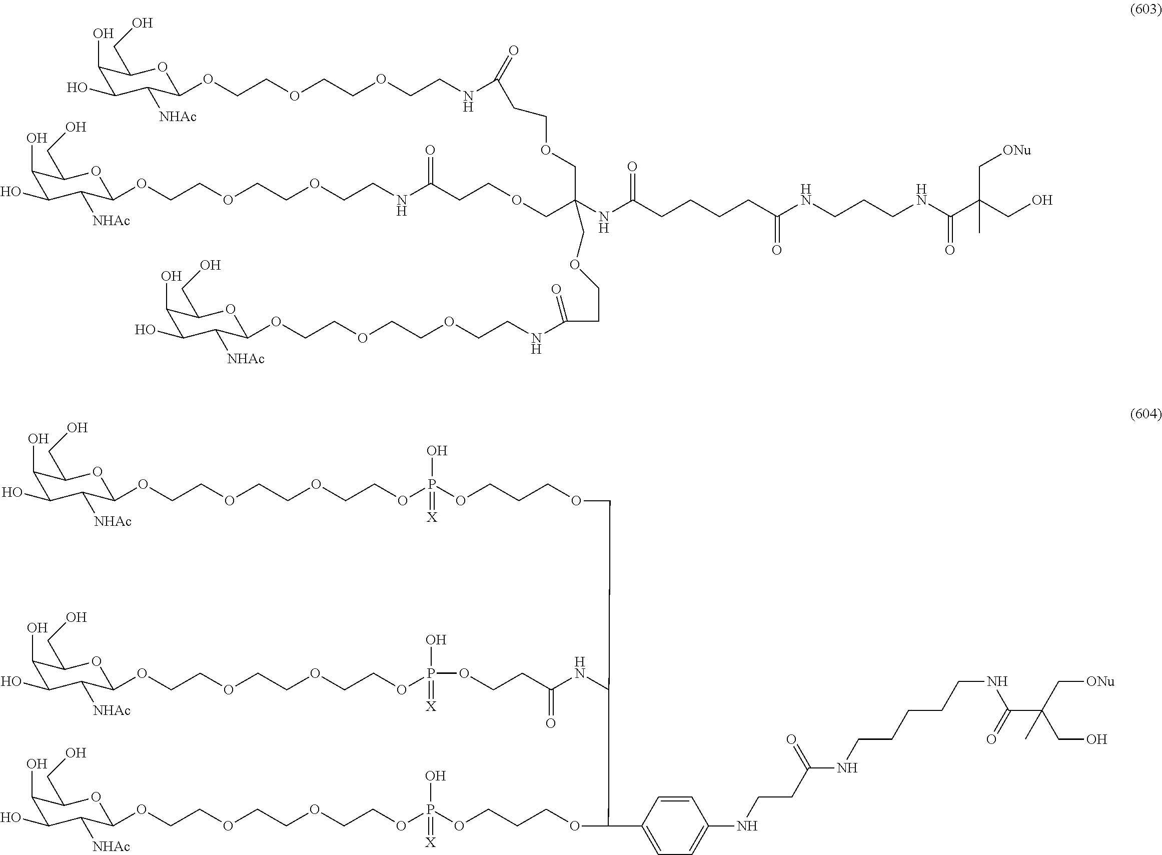 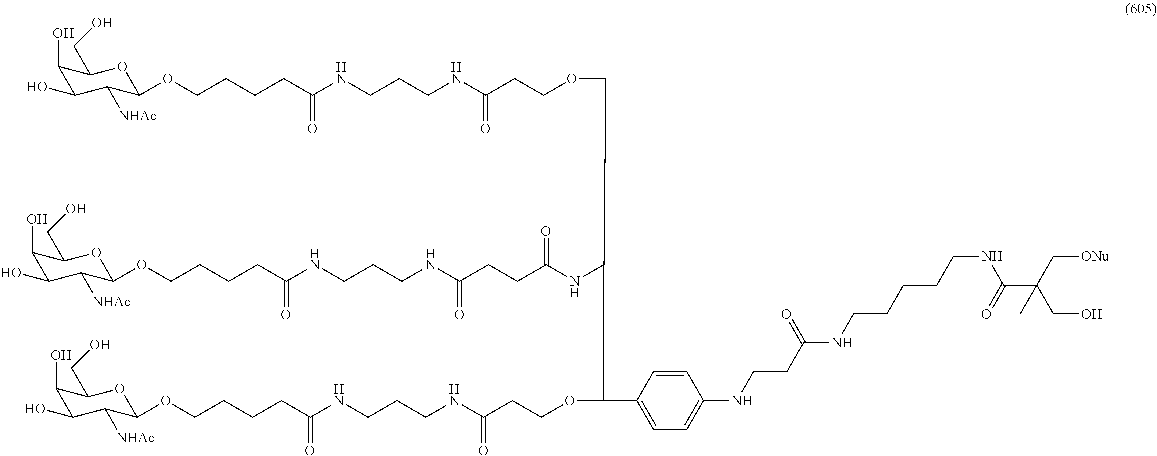 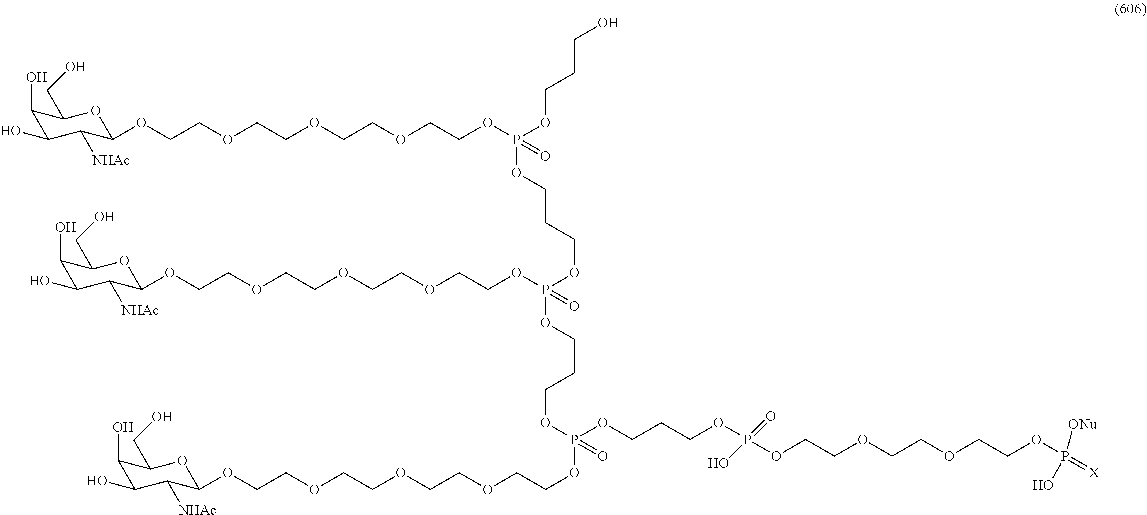 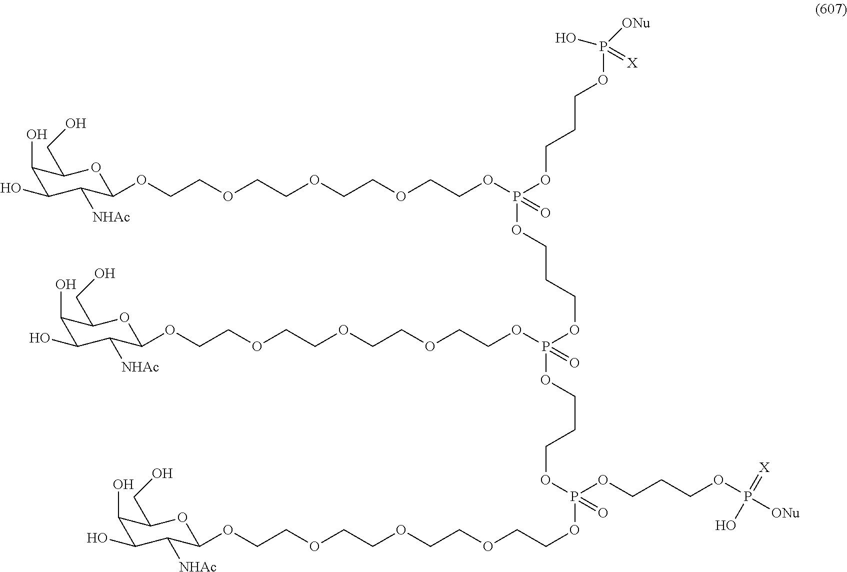
wherein X in formula (601), (602), (603), (604), (605), (606) or (607) is O or S; and Nu is any of the nucleic acid.

17. The targeted drug delivery system according to claim 16, wherein the targeted drug delivery system has a structure shown in formula (603) or (606).

18. A targeted drug delivery system according to claim 13, wherein the targeted drug delivery system comprises a ligand and the nucleic acid linked to the ligand, the ligand having a structure shown in formula (I), (II) or (V) below: 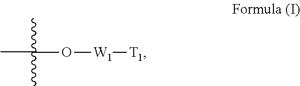
wherein the W₁ is selected from a direct linkage or 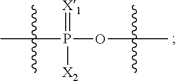
X′₁ is selected from O or S atoms, or is absent;
When X′₁ is selected from O or S atoms, X₂ is selected from —O—, —S—, —SH, —OH (hydroxy), —NH₂ (amino), C1-C6 alkyl, C1-C6 alkoxy, or —O—(CH₂)_{n′}—OR′₅, R′₅ is selected from H, a direct linkage or 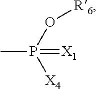
R′₆ is H or a direct linkage, X₁ is selected from an O or S atom, X₄ is —OH or —SH, and n′ is an integer from 1 to 10; when X′₁ is absent, X₂ is a direct linkage;
T₁ is selected from —(CH₂)ₘCH₃, m being an integer from 10 to 30; or 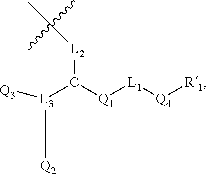
wherein Q₁ and Q₄ are each independently selected from a direct linkage, —NH₂ (amino), —COOH (carboxy), amide (—NHCO— or —CONH—), —O—, —S—, —S—S—, phosphate ester group, or phosphorothioate group;
Q₂ is selected from —SH, —OH (hydroxyl), —NH₂ (amino), —H, C1-C6 alkyl, preferably —CH₃(methyl), —COOH (carboxyl), amide group (—NHCO— or —CONH—), —O—, —S—, —S—S—, phosphoric acid ester group, phosphoric acid ester group, thiophosphoric acid ester group, or 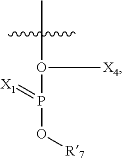
R′₇, R′₇ is H or a direct linkage, and X₁ and X₄ are as defined above;
Q₃ is selected from —H or an alkyl group of C1-C10;
L₁ is —(CH₂)ₗ—(NR′₄)ₜ—(CH₂)q-, l and q are integers 0-10 and l+q=1-10, t is 0 or 1, and R′₄ is —CO(CH₂)ᵣCOOH, and r is an integer from 10-30;
L₂ and L₃ are each independently selected from a saturated alkane chain of C1-C10 or from a direct linkage;
R′₁ is selected from a saturated fatty acid chain of C10-C30, a saturated alkane chain of C10-C30, an unsaturated hydrocarbon group of C10-C30 or —(CH₂)ₘ—X₃—R′₃, with m being an integer from 10 to 30, X₃ being selected from a direct linkage, an oxygen atom or a sulfur atom, R′₃ is chosen from a saturated six-membered heterocyclic group containing nitrogen and oxygen, H, a direct linkage, or 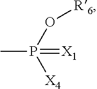
R′₆, X₁ and X₄ are as defined above; when X₃ is a direct linkage, R′₃ is neither an H nor a direct linkage;
T₁ is not —(CH₂)ₘCH₃ when W₁ is a direct linkage; 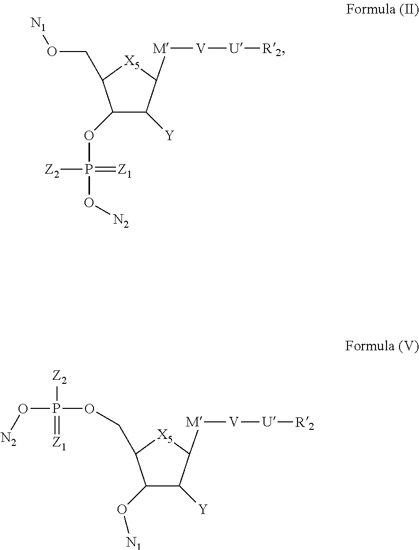
wherein for Formula (II) and (V), the five-membered ring is a five-membered cyclic sugar structure in ribose or deoxyribose, wherein X₅ is selected from —CH₂—, —CH(CH₃)—, —C(CH₃)₂—, —O—, —NH—, —N(CH₃)— or —S—;
the M′ is selected from H, —O—, —C— or a modified or unmodified nucleotide base;
the N₁ is selected from a direct linkage, H, an alkyl group of C1-C3 or 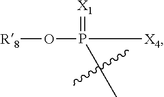
R′₈ is H or a direct linkage and the X₁ and X₄ are as defined above;
N₂ is selected from a direct linkage, H or an alkyl group of C1-C3;
Y is selected from H, NH₂, OH, halogen, C1-C6 alkyl, C1-C6 haloalkyl, —O—R′₉ or —O—(CH₂)ₙ—O—R′₁₀, with R′₉ being C1-C6 alkyl; preferably —O—CH₃, n is an integer from 1 to 6, R′₁₀ is C1-C6 alkyl, preferably n is 2, R′₁₀ is C1 alkyl, i.e. 2′-methoxyethoxy;
V is selected from a saturated alkane chain of C1-C4 or is absent;
U′ is selected from —NH₂ (amino), —COOH (carboxyl), amide (—NHCO— or —CONH—) or absent;
Z₁ is selected from O or S atoms;
Z₂ is selected from C10-C30 alkoxy, fatty acid chains; preferably terminal carboxy fatty acids, amide lipid chains, olefinic chains, or alkane chains;
R′₂ is selected from C10-C30 alkoxy, fatty acid chains; preferably terminal carboxylated fatty acids, amidolipid chains, olefinic chains, alkane chains, or absent.

19. The targeted drug delivery system according to claim 18, wherein the ligand is selected from at least one of the following structures (L1)-(L36) or (L′10): 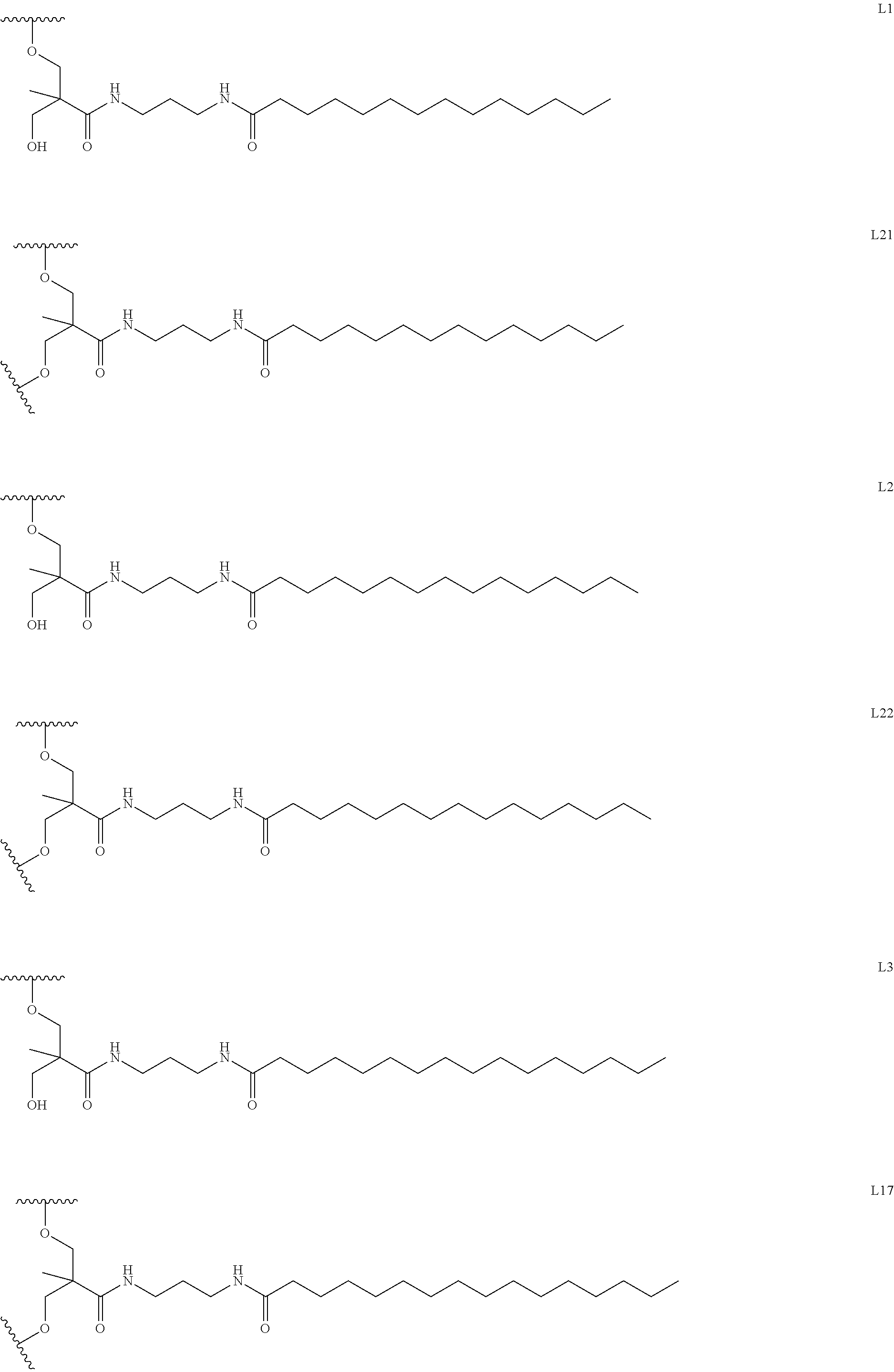 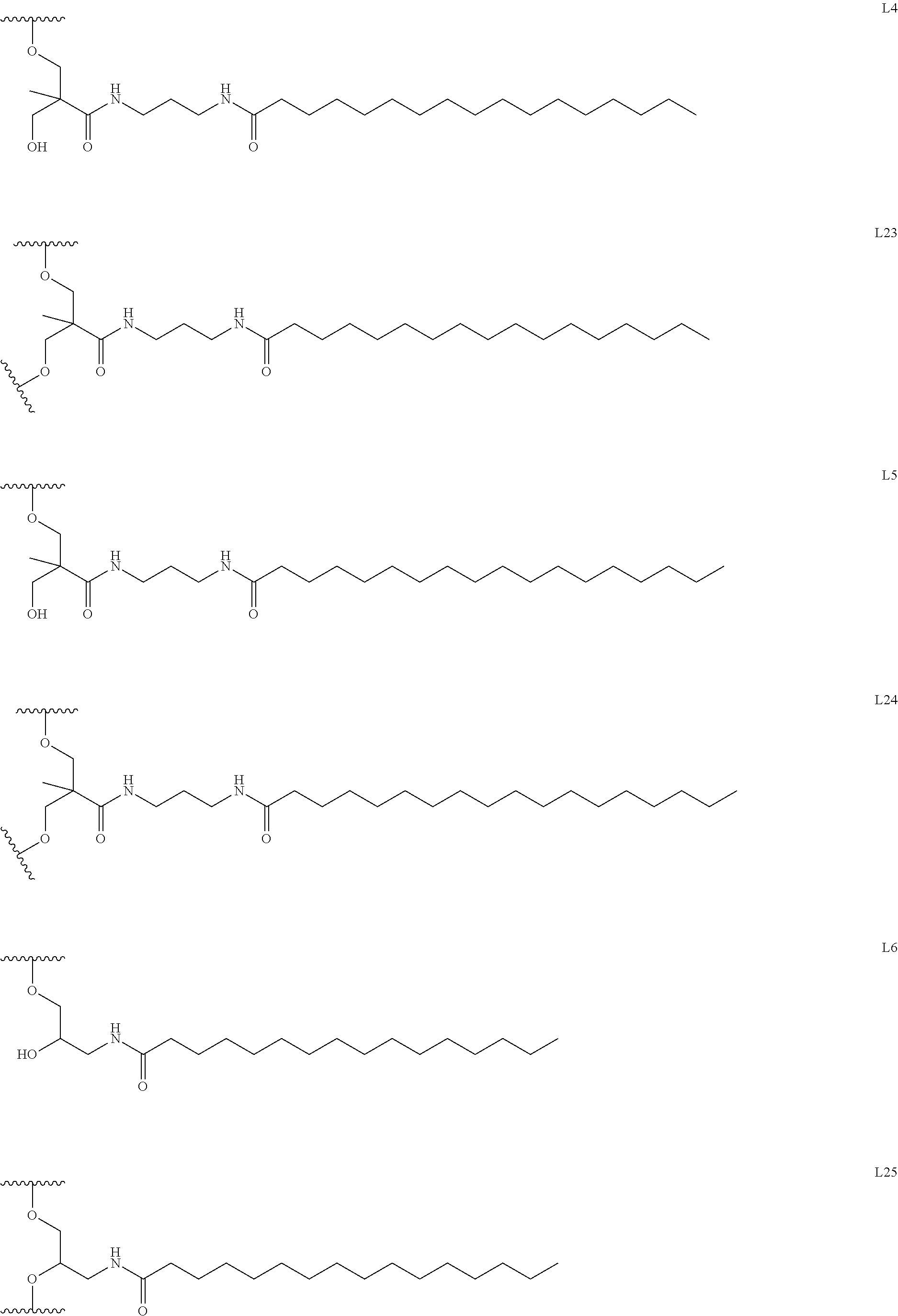 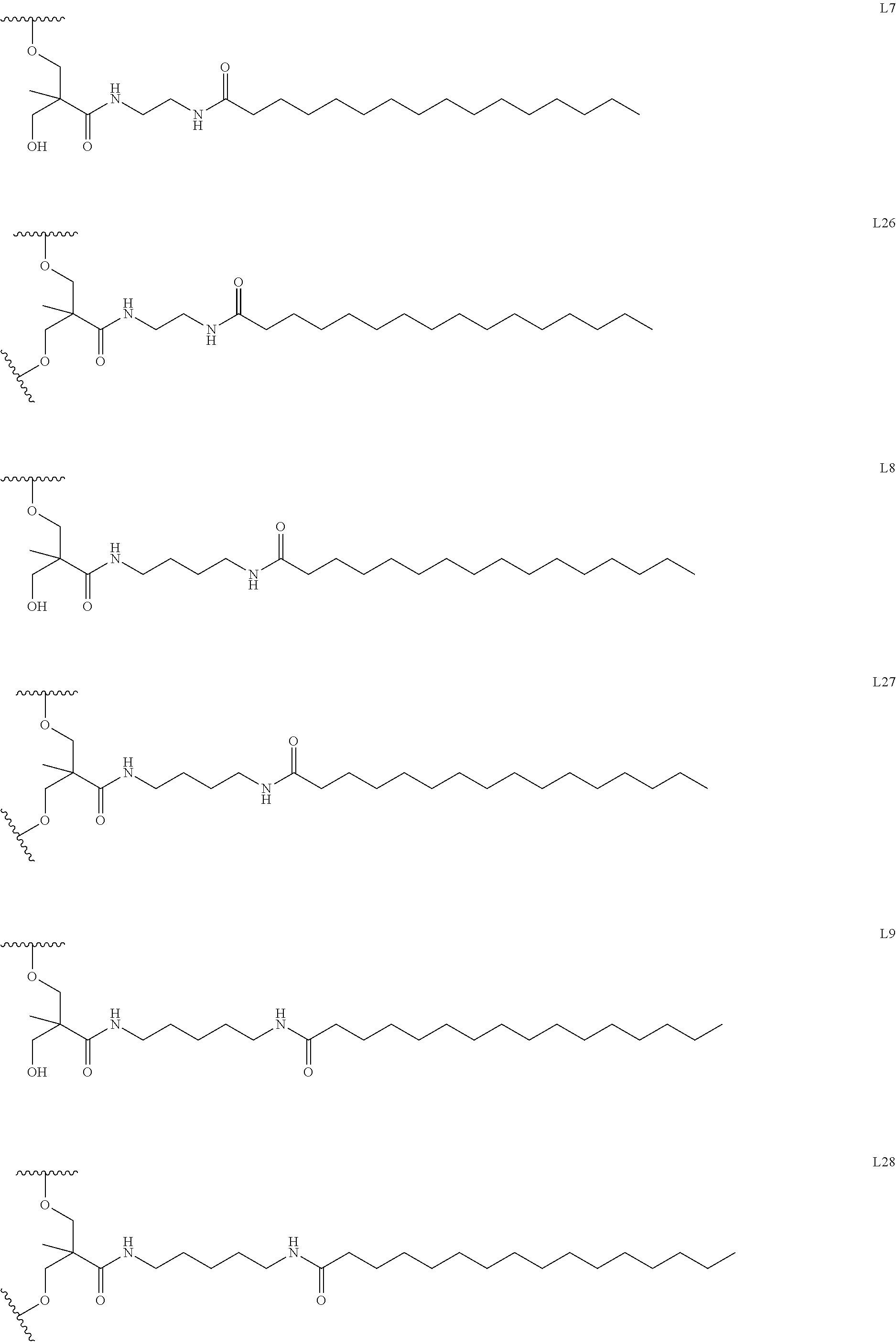 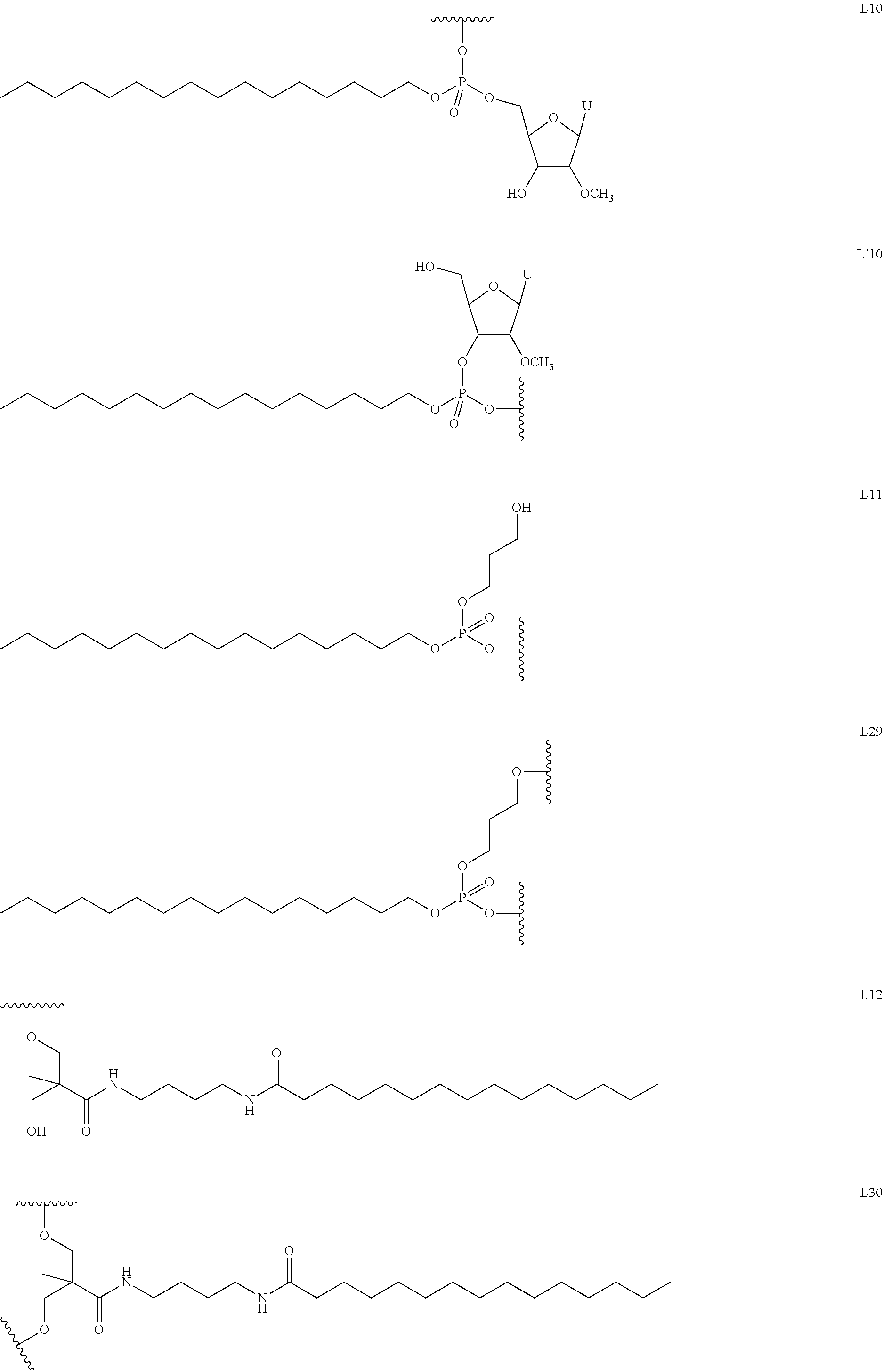 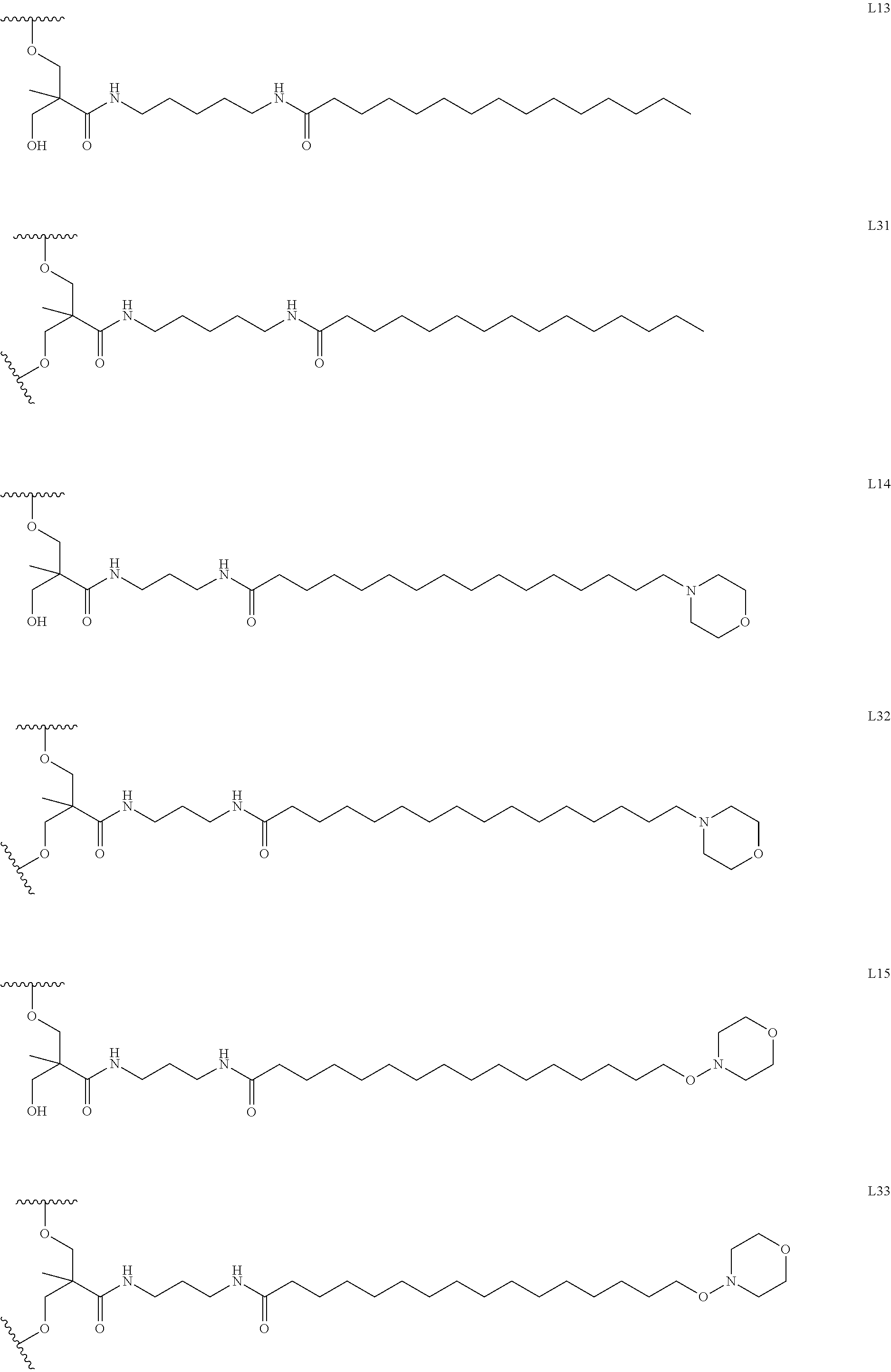 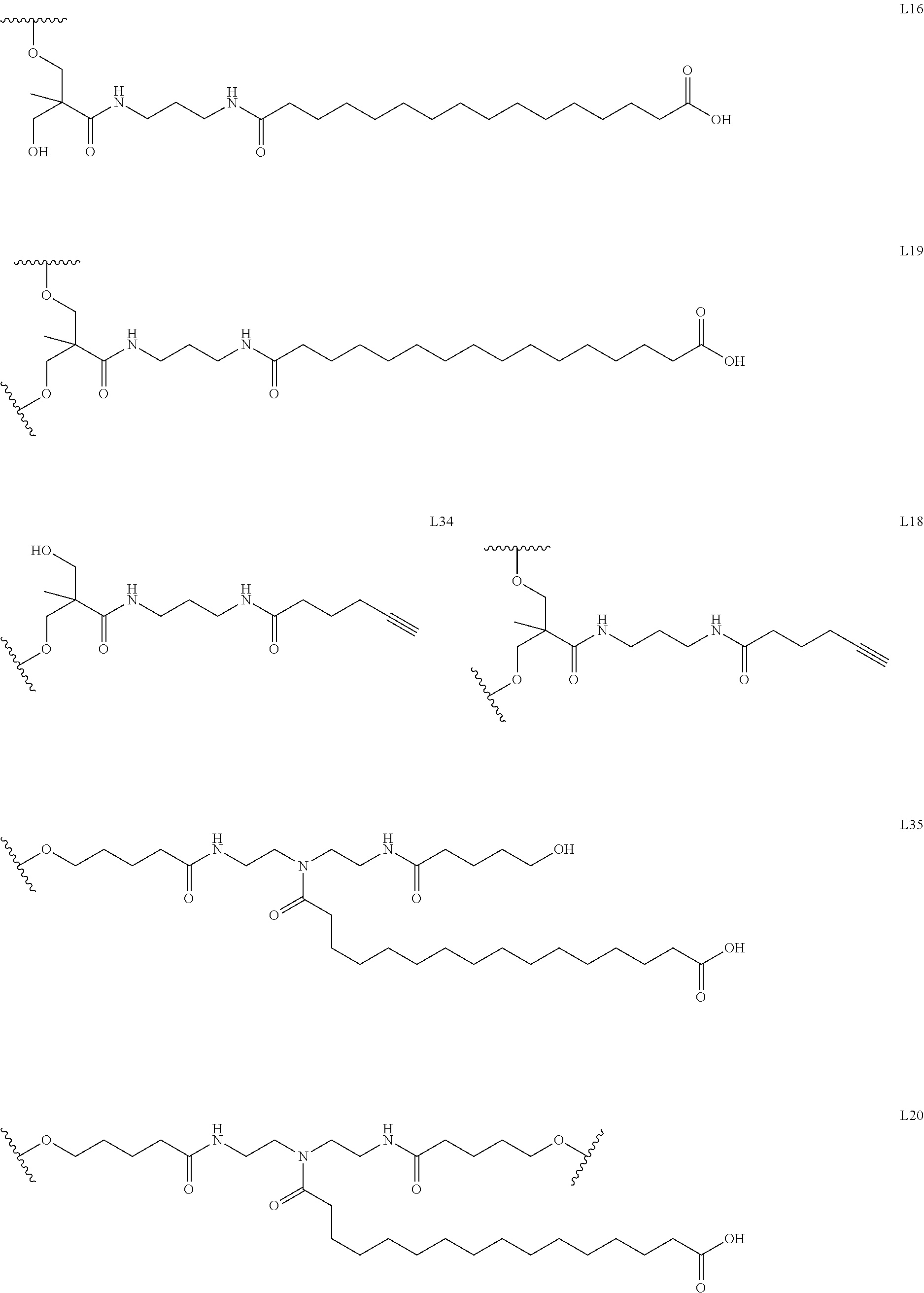 [SMILES: *OCC(C)(CO)C(=O)NCCCNC(=O)CCCCCCCCCCCCCCC(=O)O ; *OCC(C)(CO*)C(=O)NCCCNC(=O)CCCCCCCCCCCCCCC(=O)O ; *OCC(C)(CO)C(=O)NCCCNC(=O)CCCC#C ; *OCC(C)(CO*)C(=O)NCCCNC(=O)CCCC#C ; *OCCCCC(=O)NCCN(CCNC(=O)CCCCO)C(=O)CCCCCCCCCCCCCCC(=O)O ; *OCCCCC(=O)NCCN(CCNC(=O)CCCCO*)C(=O)CCCCCCCCCCCCCCC(=O)O] 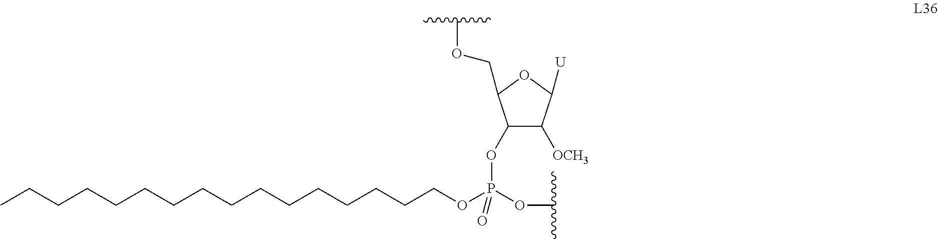
wherein U is

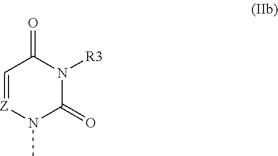

20. The targeted drug delivery system according to claim 18, wherein the ligand is selected from at least one of the following structures (L1′)-(L36′) or (L′10′): 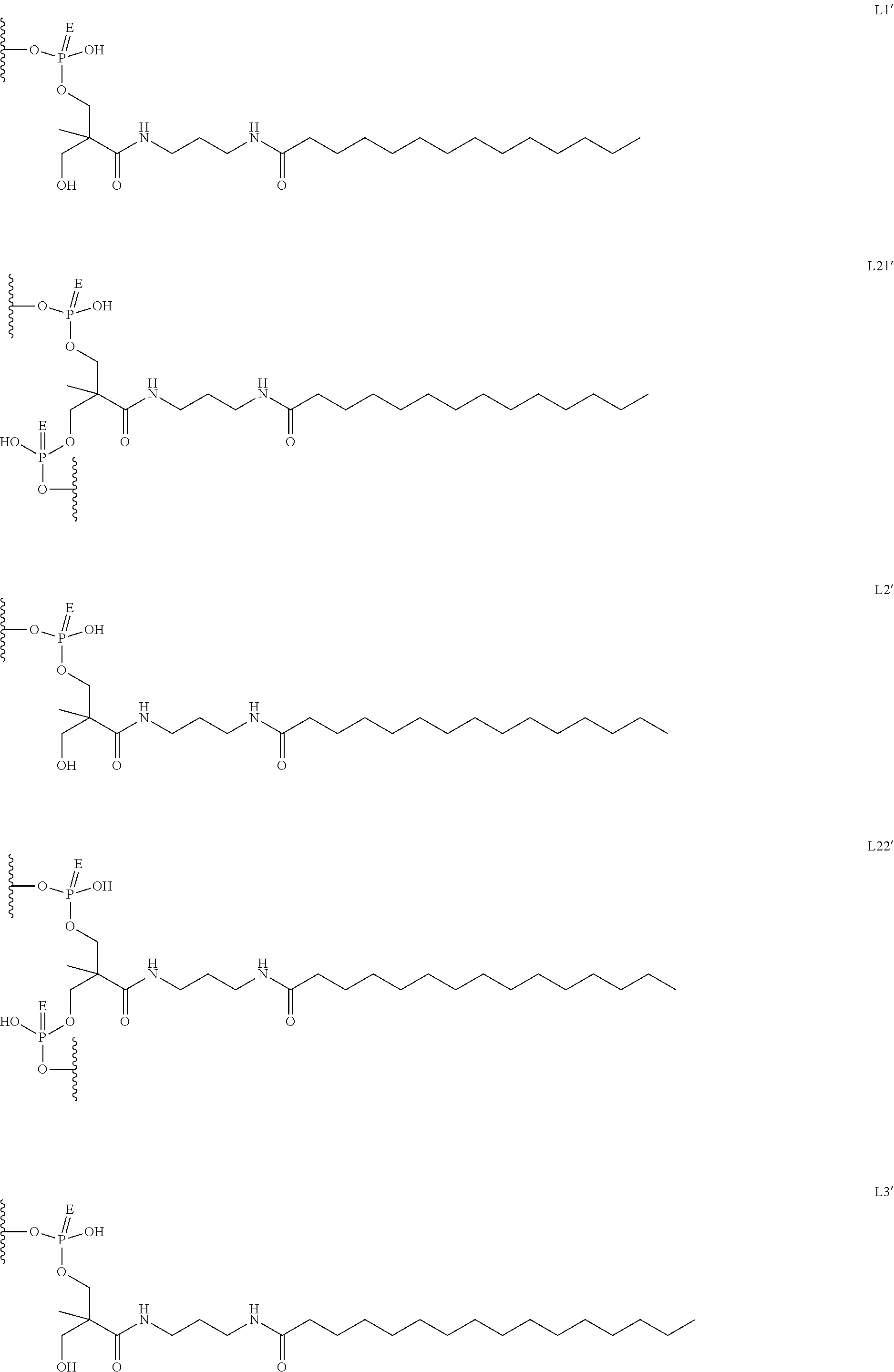 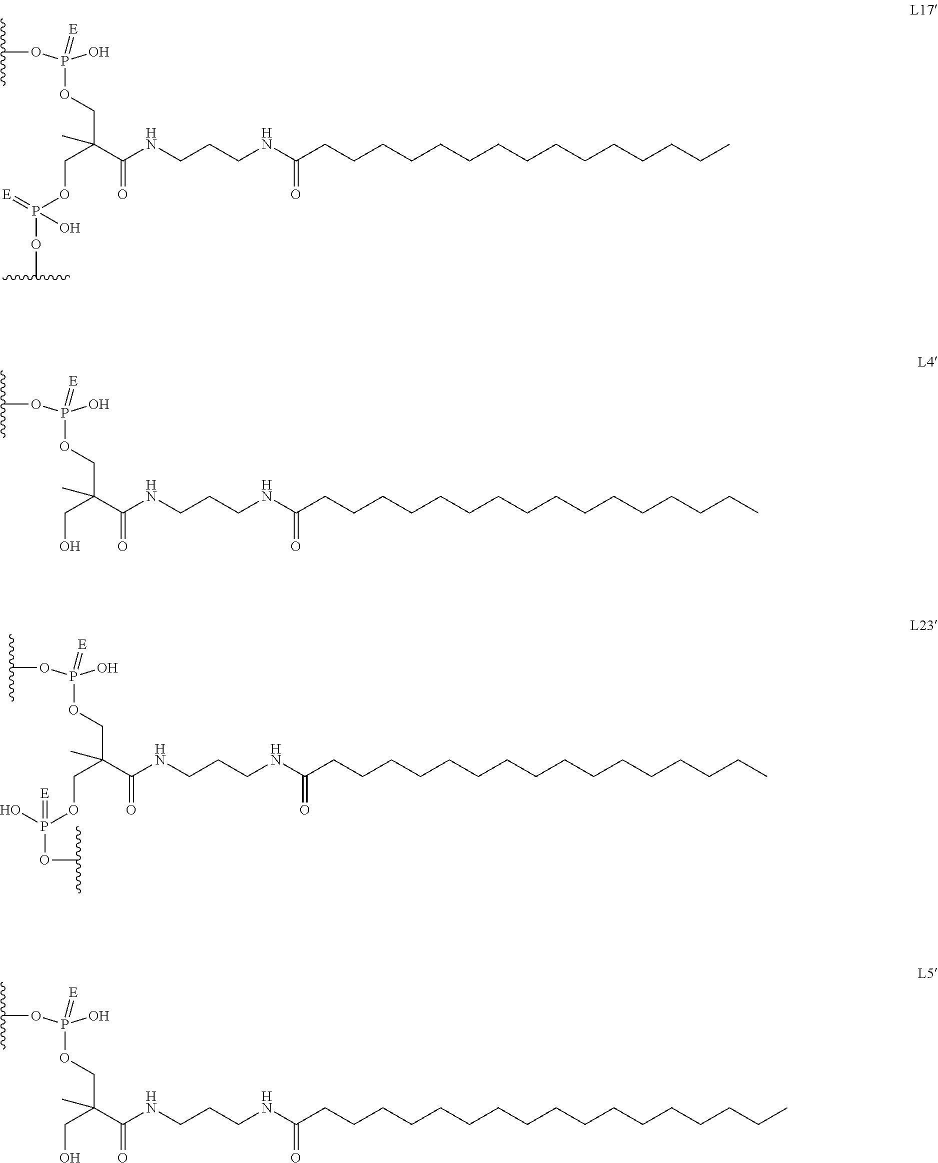 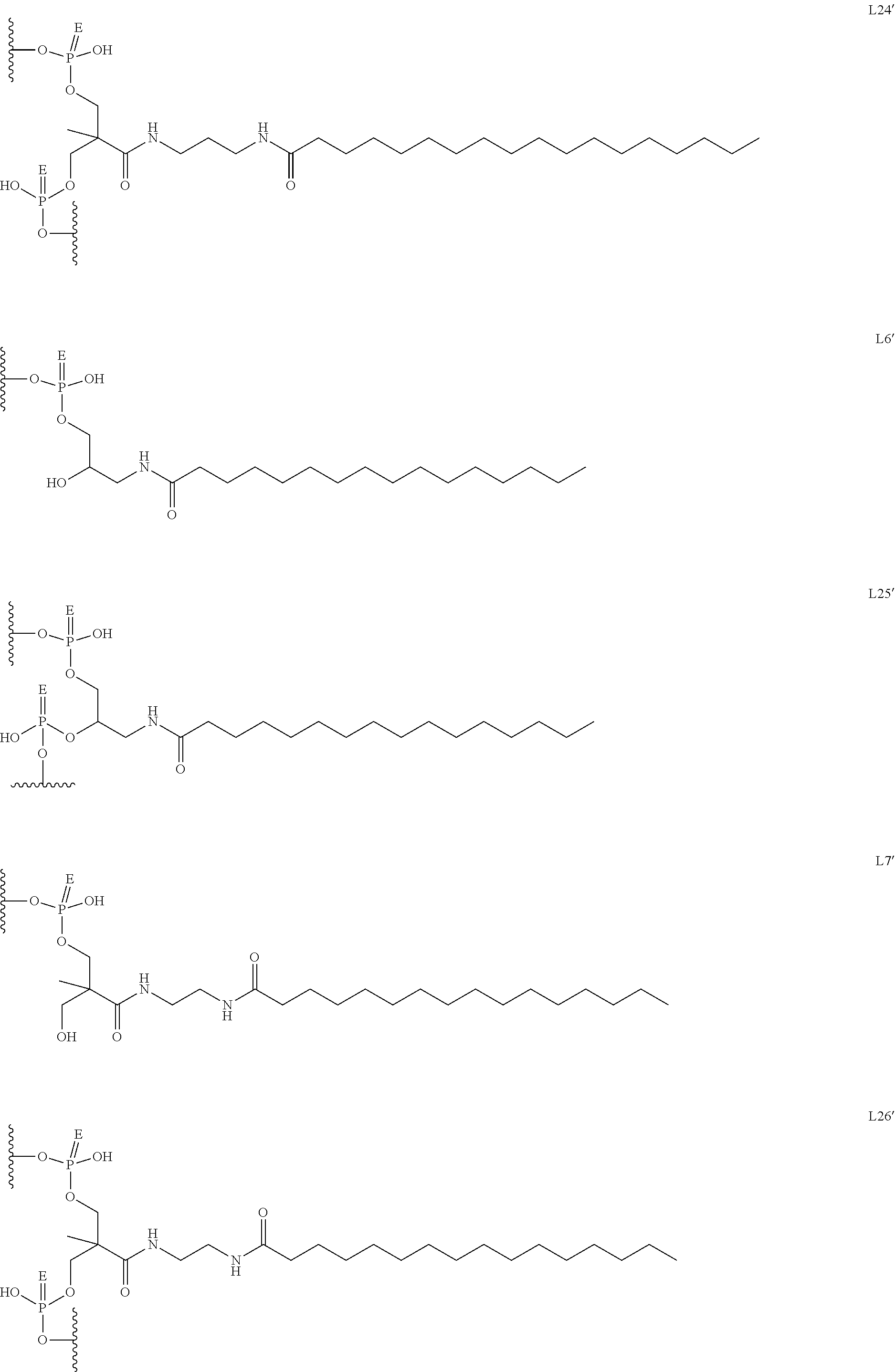 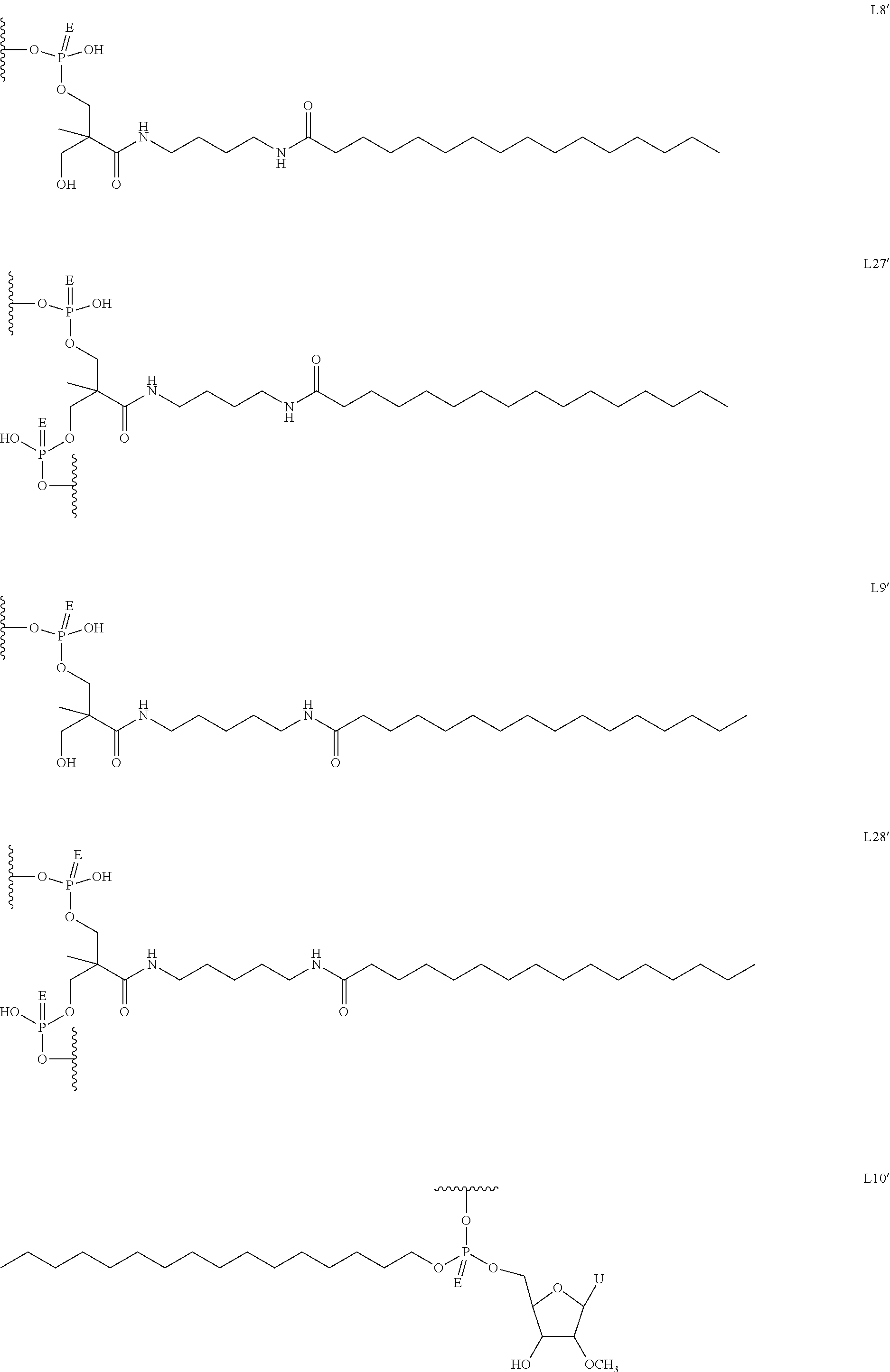 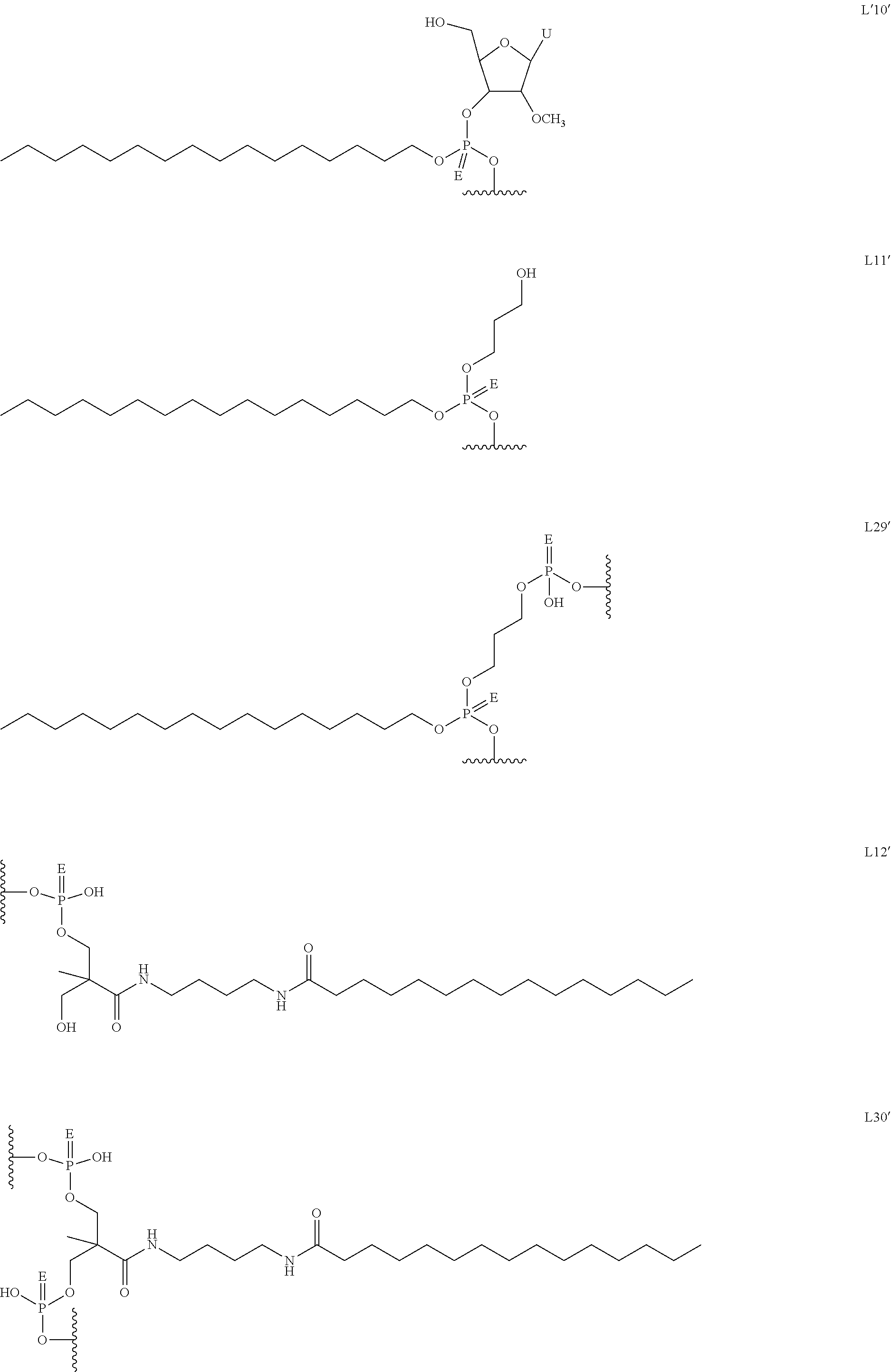 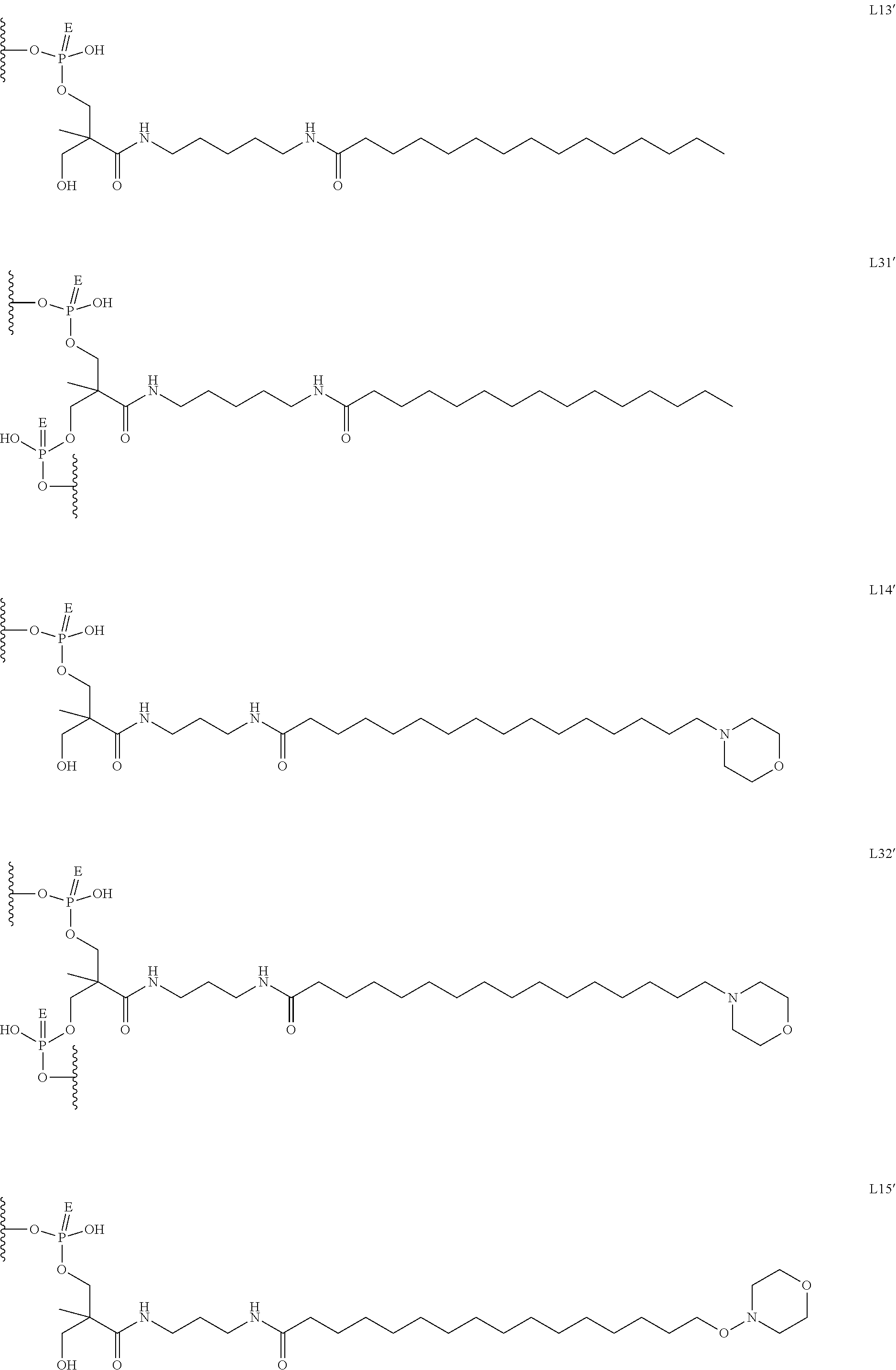 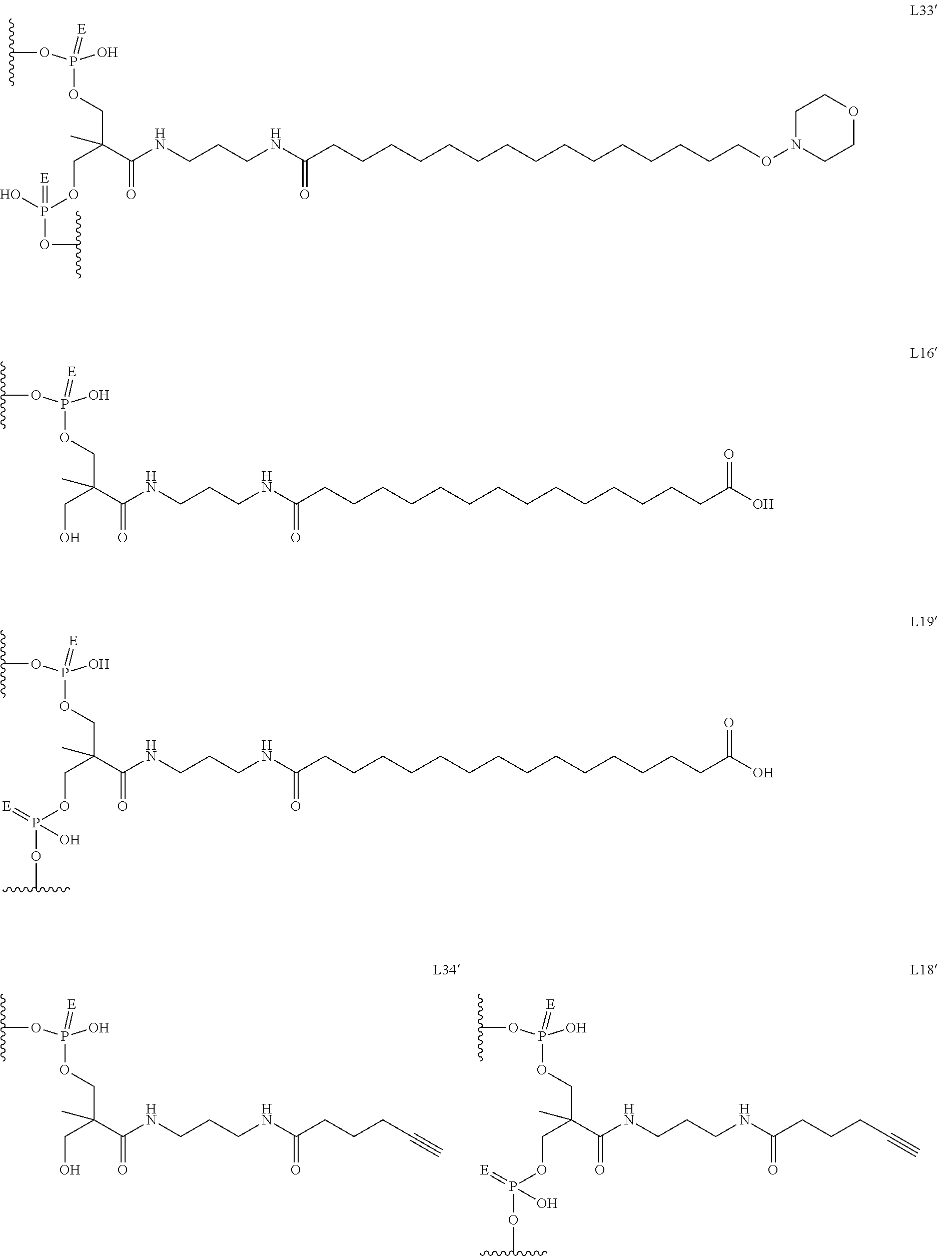 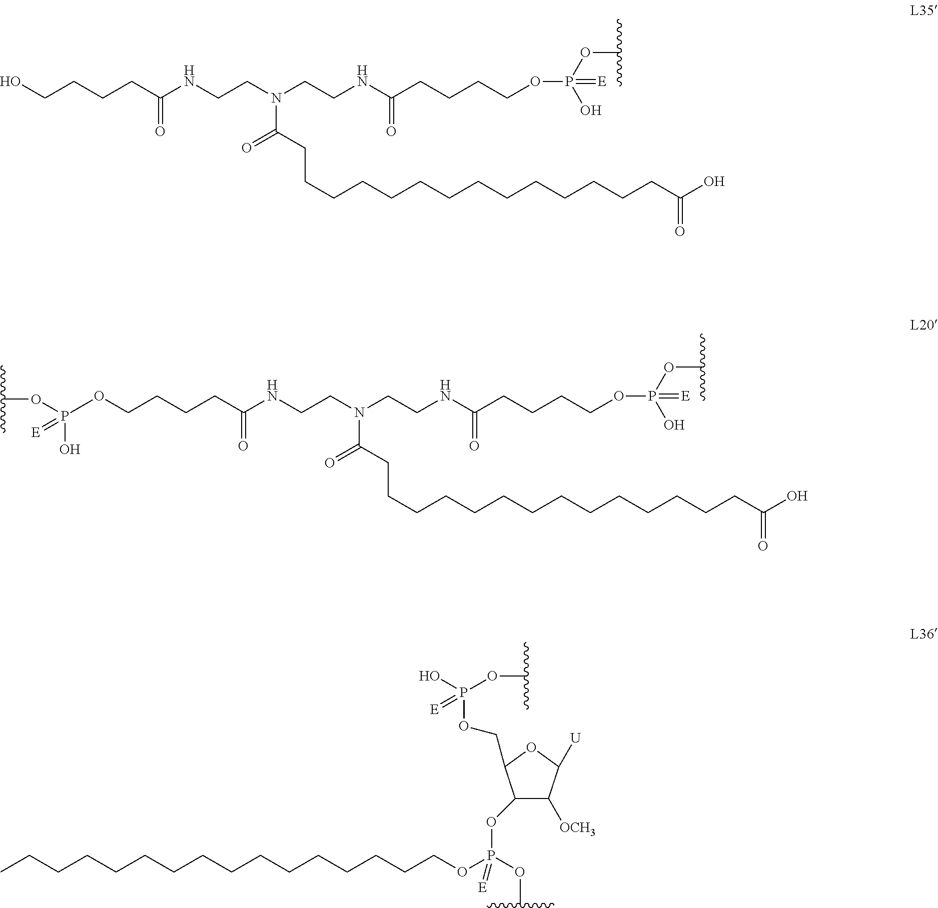
wherein E is selected from O and S and U is

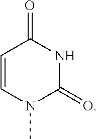

21. The targeted drug delivery system according to claim 18, wherein the targeted drug delivery system comprises a structure shown in Formula (III), Formula (IV), or Formula (VI): 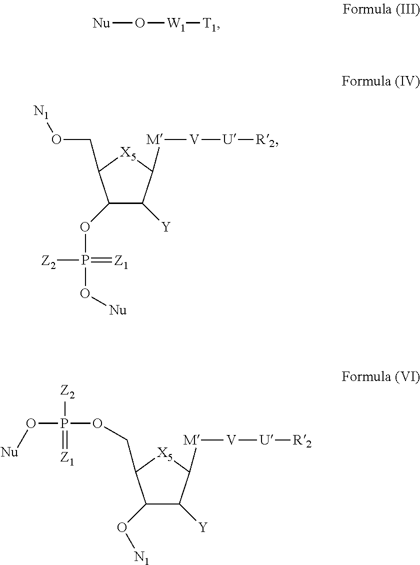
wherein Nu is a nucleic acid and other variables are as defined in claim 18.

22. The targeted drug delivery system according to claim 21, wherein the targeted drug delivery system has a structure of Formula (III); and
1) X₂ is —O—(CH₂)_{n′}—OR′₅, R′₅ is selected from a direct linkage or 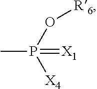
R′₆ is a direct linkage, n′ is an integer from 1 to 10; or
2) Q₂ is selected from —O—, —S— or 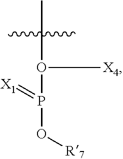
R′₇ is selected from a direct linkage; or
3) R′₁ is selected from —(CH₂)ₘ—X₃—R′₃, m is an integer from 10 to 30, X₃ is selected from an oxygen atom, a sulfur atom, a direct linkage or 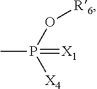
and R′₆ is a direct linkage.

23. The targeted drug delivery system according to claim 21, wherein the targeted drug delivery system has a structure of Formula (IV) or Formula (VI), wherein N₁ is a direct linkage or 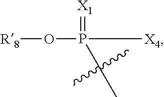
and R′₈ is a direct linkage.

24. An isolated cell, wherein the isolated cell comprises a nucleic acid according to claim 1.

25. A pharmaceutical composition, wherein the pharmaceutical composition comprises a nucleic acid according to claim 1 or a targeted drug delivery system thereof and a pharmaceutically acceptable carrier.

26. A method of inhibiting MAPT expression in a cell, the method comprising: contacting the cell with a nucleic acid according to claim 1, a targeted drug delivery system thereof, or a pharmaceutical composition thereof to inhibit MAPT expression in the cell.

27. A method of treating and/or preventing a disease associated with MAPT, the method comprising administering a nucleic acid according to claim 1, a targeted drug delivery system thereof, or a pharmaceutical composition thereof.

28. The method according to claim 27, wherein the disease is:
(i) a disease associated with enhanced or elevated MAPT; or
(ii) a disease that would benefit from reducing MAPT expression.

29. The method according to claim 27, wherein the disease is selected from one or more of: alzheimer's disease (AD), frontotemporal dementia (FTD), progressive supranuclear palsy (PSP), corticobasal ganglionic degeneration (CBD), Pickle's disease (PiD), agranulocytosis granulomatosa (AGD), subacute sclerosing proptosis total encephalitis (SSPE), Christiansen's syndrome (CS), post-encephalitis Parkinson's syndrome (PEP), Guadeloupe Parkinson's syndrome (GP), globoid glial cell tauopathy (GGT), spinal cerebellar ataxia type 11 (SCA11), chronic traumatic encephalopathy (CTE), aging-associated tau astrocytosis (ARTAG), and primary age-related tauopathy (PART).

30. The method according to claim 27, wherein the nucleic acid, the targeted drug delivery system, or the pharmaceutical composition is administered to a subject by subcutaneous administration, intravenously administration, intramuscular administration, and/or intracranial administration.

31. An isolated cell, wherein the isolated cell comprises a targeted drug delivery system according to claim 13.
